(19) United States
(12) Patent Application Publication
(10) Pub. No.: US 2024/0342246 A1
(43) Pub. Date: Oct. 17, 2024

(54) INTERLEUKIN-12 VARIANTS AND METHODS OF USE

(71) Applicant: Yale University, New Haven, CT (US)

(72) Inventors: Aaron Ring, New Haven, CT (US); John Huck, New Haven, CT (US)

(21) Appl. No.: 18/683,398

(22) PCT Filed: Aug. 16, 2022
(86) PCT No.: PCT/US22/75002

§ 371 Date: Feb. 13, 2024

Related U.S. Application Data
(60) Provisional application No. 63/233,511, filed on Aug. 16, 2021.

(51) Int. Cl. A61K 38/20; A61K 45/06; A61K 47/60; A61K 47/64; A61K 47/68; A61P 35/00

(52) U.S. Cl. CPC A61K 38/208; A61K 45/06; A61K 47/60; A61K 47/643; A61K 47/6843; A61P 35/00

(57) ABSTRACT

The present disclosure provides compositions and methods comprising variant polypeptides of IL-12 with partial agonism relative to wild-type IL-12 for use in therapeutic and non-therapeutic applications.

### CROSS-REFERENCE TO RELATED APPLICATIONS

This application claims priority to and the benefit of U.S. Provisional Application No. 63/233,511, filed Aug. 16, 2021, the disclosure of which is incorporated herein by reference in its entirety.

### STATEMENT REGARDING FEDERALLY SPONSORED RESEARCH OR DEVELOPMENT

This invention was made with government support under grant number U01 CA233096 awarded by the National Institutes of Health. The government has certain rights in the invention.

### BACKGROUND

Interleukin-12 (IL-12) orchestrates Th1 immunity and powerfully induces secretion of IFN-γ by NK cells and cytotoxic T-cells, making it an attractive candidate for cancer immunotherapy. Administration of IL-12 has shown tremendous promise in stimulating anti-tumor responses pre-clinically, but has not translated in humans due to unacceptable toxicities and a narrow therapeutic index. The full therapeutic potential of IL-12 may be limited by its pleiotropic nature, leading to activation of multiple cell types that promote beneficial and adverse effects.

Thus, there is a need in the art for improved compositions and methods that provide IL-12 with minimal toxicity and a broad therapeutic index to treat and prevent cancer and other diseases and disorders. This disclosure satisfies this unmet need.

### SUMMARY

In one embodiment, the present disclosure relates to a composition comprising an IL-12 variant polypeptide, wherein the IL-12 variant polypeptide possesses sub-maximal signaling efficacy through its receptors relative to wild-type (WT) IL-12. In one embodiment, the IL-12 variant polypeptide comprises at least one mutation relative to WT IL-12. In one embodiment, the IL-12 variant polypeptide comprises a p35 subunit (IL-12p35) with or without a signal peptide and a p40 subunit (IL-12p40) with or without a signal peptide. In one embodiment, said IL-12p40 of the IL-12 variant polypeptide comprises at least one mutation selected from the group consisting of: H216X, K217X, and K219X, relative to SEQ ID NO: 1. In one embodiment, said IL-12p40 of the IL-12 variant polypeptide comprises at least one mutation selected from the group consisting of: H216A, K217A, and K219A, relative to SEQ ID NO: 1. In one embodiment, said IL-12p40 of the IL-12 variant polypeptide comprises an amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 34.

In one embodiment of the composition of the disclosure, said IL-12p35 of the IL-12 variant polypeptide comprises an amino acid sequence selected from the group consisting of SEQ ID NO: 2, SEQ ID NO: 30 and SEQ ID NO: 35.

In one embodiment of the composition of the disclosure, said IL-12p40, said IL-12p35, or a combination thereof is fused to at least one in vivo half-life extending fusion selected from the group consisting of: an IgG Fc domain, an IgG Fc variant domain, human serum albumin (HSA), polyethylene glycol (PEG), and an anti-HSA nanobody.

In one embodiment of the composition of the disclosure, said IgG Fc domain comprises a human IgG1 domain comprising an amino acid sequence of SEQ ID NO: 10, and wherein said IgG Fc variant domain comprises at least one selected from the group consisting of: a human IgG1 Fc “knob” domain comprising an amino acid sequence of SEQ ID NO: 14; and a human IgG1 Fc “hole” domain comprising an amino acid sequence of SEQ ID NO: 14. In one embodiment, the IL-12 variant polypeptide comprises a bivalent homodimeric IgG Fc comprising at least two human IgG1 Fc domains. In one embodiment, the IL-12 variant polypeptide comprises a bispecific heterodimeric IgG Fc comprising at least one human IgG1 Fc “knob” and at least one IgG Fc “hole”. In one embodiment, the IL-12 variant polypeptide comprises a single chain bivalent homodimeric IgG Fc comprising said IL-12p40 fused to said IL-p35 via a linker and at least two human IgG1 Fc domains. In one embodiment, the IL-12 variant polypeptide comprises a single chain monomeric IL-12 comprising IL-12p40 fused to IL-p35 via a linker and a bispecific heterodimeric IgG Fc comprising at least one human IgG1 Fc “knob” and at least one IgG Fc “hole”. In one embodiment, the IL-12 variant polypeptide comprises dimeric IL-12 comprising said IL-12p40 and said IL-p35 and a bispecific heterodimeric IgG Fc comprising at least one human IgG1 Fc “knob” and at least one IgG Fc “hole.

In one embodiment, the present disclosure relates to a composition comprising one or more nucleic acid molecule encoding at least one IL-12p40 peptide selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, and SEQ ID NO: 9, and SEQ ID NO: 34. In one embodiment, the composition further comprises a nucleic acid molecule encoding IL-12p35 peptide comprising an amino acid sequence selected from the group consisting of SEQ ID NO: 2, SEQ ID NO: 30 and SEQ ID NO: 35.

In one embodiment of the composition of the disclosure, said nucleic acid encoding said IL-12p40, said nucleic acid encoding said IL-12p35, or a combination thereof, further encodes a nucleic acid sequence encoding an in vivo half-life extending fusion selected from the group consisting of: an IgG Fc domain, an IgG Fc variant domain, human serum albumin (HSA), polyethylene glycol (PEG), and an anti-HSA nanobody.

In one embodiment, the present disclosure relates to a method of treating or preventing a disease or disorder in a subject in need thereof, comprising administering to the subject a composition comprising an IL-12 variant polypeptide, wherein said IL-12 variant polypeptide possesses sub-maximal signaling efficacy through its receptors relative to WT IL-12.

In one embodiment of the method the disclosure, the IL-12 variant polypeptide comprises a p35 subunit (IL-12p35) and a p40 subunit (IL-12p40). In one embodiment, said IL-12p40 of the IL-12 variant polypeptide comprises an amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34. In one embodiment, said IL-12p35 of the IL-12 variant polypeptide comprises an amino acid sequence selected from the group consisting of SEQ ID NO: 2, SEQ ID NO: 30, and SEQ ID NO: 35. In one embodiment, said IL-12p40, said IL-12p35, or a combination thereof is fused to at least one in vivo half-life extending fusion selected from the group consisting of: an IgG Fc domain, an IgG Fc variant domain, human serum albumin (HSA), polyethylene glycol (PEG), and an anti-HSA nanobody. In one embodiment, said disease or disorder is cancer.

In one embodiment, the method further comprises administering to the subject at least one additional agent selected from the group consisting of: a chemical compound, a polypeptide, a peptide, a peptidomimetic, an antibody, a cytokine, a nucleic acid molecule (e.g., mRNA), a ribozyme, a small molecule chemical compound, and an antisense nucleic acid molecule. In one embodiment, said at least one additional agent comprises one or more selected from the group consisting of: a cancer therapeutic agent or cancer immunotherapeutic agent.

In one embodiment, the method of the disclosure comprises administering said IL-12 variant polypeptide via one or more mechanism selected from the group consisting of: a) a lipid nanoparticle encapsulated mRNA molecule encoding said IL-12 variant polypeptide; b) a viral vector expressing said IL-12 variant polypeptide; and c) an engineered immune cell expressing said IL-12 variant polypeptide. In one embodiment, said administration comprises one or more selected from the group consisting of: a) Systemic administration; and b) Local administration to at least one specific tissue.

### BRIEF DESCRIPTION OF THE DRAWINGS

The following detailed description of embodiments of the disclosure will be better understood when read in conjunction with the appended drawings. It should be understood that the disclosure is not limited to the precise arrangements and instrumentalities of the embodiments shown in the drawings.

FIG. 1A and FIG. 1B, depict models that predict residues at the IL-12Rβ1:IL-12p40 interface. FIG. 1A depicts a zoomed in model of IL-12p40 in complex with a neutralizing nanobody. Residues predicted to mediate interactions with the nanobody, and thus likely with IL-12Rβ1 as well, are depicted in blue (see written labels for color), IL-12p40 is depicted in green, and the nanobody is depicted in pale yellow. FIG. 1B depicts a schematic of the predicted interactions between IL-12, comprising IL-12p40 and IL-12p35 subunits, and IL-12Rβ2 and IL-12Rβ1. The gray “X” indicates the interface between IL-12Rβ1 and IL-12p40 that, if selectively disrupted, may reduce the recruitment of IL-12Rβ1 to the IL-12:IL-12Rβ2 complex and attenuate downstream STAT4 signaling.

FIG. 2A and FIG. 2B, depict exemplary results demonstrating protein expression and purification of wild-type (WT) IL-12 and mutant variants. FIG. 2A depicts exemplary results demonstrating comparable expression and mobility of WT and mutant IL-12 proteins. Proteins were separated on an SDS-PAGE gel and stained with Coomassie Brilliant Blue. FIG. 2B depicts exemplary results demonstrating purification of IL-12 and mutant variants. Proteins were isolated by nickel-NTA affinity chromatography and then further purified by size exclusion chromatography.

FIG. 3A through FIG. 3D depict exemplary results demonstrating a graded response relative to WT depending upon the number and nature of the interface residues mutated to alanine residues. FIG. 3A depicts exemplary results demonstrating the reduced agonism of H216A/K217A/K219A triple mutant (HKK) as compared to WT IL-12. FIG. 3B depicts exemplary results demonstrating that agonism is reduced to a lesser extent in the H216A/K219A double mutant as compared to WT. FIG. 3C depicts exemplary results of single-point responses to all variants as compared to WT IL-12 and unstimulated cells, demonstrating a graded response that can be tailored to the particular level of agonism desired. FIG. 3D depicts the results of 3C as a percentage of agonism relative to WT. In all cases, human NK cells were stimulated with IL-12, a mutant variant, or left unstimulated, and phosphorylated STAT4 was measured by flow cytometry as a measure of IL-12 agonism.

FIG. 4A through FIG. 4B depict exemplary results of IL-12 expressed as a bispecific heterodimeric Fc-fusion protein, a method of prolonging the in vivo half-life. FIG. 4A depicts a schematic of the bispecific heterodimeric Fc-fusion protein of IL-12 tested for IL-12 agonism. FIG. 4B depicts exemplary results demonstrating that the bispecific heterodimeric Fc-fusion protein of IL-12 unexpectedly has attenuated agonism as compared to WT IL-12, but not as profoundly attenuated as the HKK triple mutant as described in FIGS. 3A to 3D. Phosphorylated STAT4 was measured by flow cytometry as in FIGS. 3A to 3D.

FIG. 5A through FIG. 5D depict exemplary methods of employing Fc-fusion IL-12 variants to prolong the half-life of partial IL-12 agonists of the present disclosure. FIG. 5A depicts a schematic of bivalent Fc fused to either p40 or p35 and co-expressed with the corresponding subunit to generate dimeric IL-12. FIG. 5B depicts a schematic of bivalent Fc fused to p35 which in turn is fused to p40 and is expressed as a single chain construct to form dimeric IL-12. FIG. 5C depicts a schematic of bispecific Fc “knob” fused to p35 which in turn is fused to p40, expressed as a single chain construct, and co-expressed with the corresponding bispecific Fc “hole” to form monomeric IL-12. FIG. 5D depicts a schematic of bispecific Fc “knob” fused to either p40 or p35 and co-expressed with the corresponding subunit and the corresponding bispecific Fc “hole” to form monomeric IL-12.

FIG. 6A through FIG. 6B depict exemplary results demonstrating further reduced agonism of Fc-fusion of select Fc-fusion variants. FIG. 6A depicts exemplary results of single-point responses to Fc-fusion variants as compared to WT IL-12 and IL-12 H216A/K217A/K219A triple mutant (HKK), demonstrating that the graded response as shown in FIGS. 3A to 3D can be further tuned via Fc-fusions. FIG. 6B depicts the effects of WT IL-12, IL-12 HKK and Fc-fusion variants on the phosphorylation of STAT4 in human NK cells in response to titrated amounts of supernatant. Proteins were expressed in Expi293 cells and cell culture supernatants containing the secreted proteins was used to stimulate NK cells. The x-axis depicts a titration of cell culture supernatants shown as a percentage of the total volume used to stimulate the NK cells.

FIG. 7 depicts a schematic of additional exemplary methods of prolonging the in vivo half-life of a partial IL-12 agonist of the present disclosure. Exemplary methods include, but are not limited to, fusion to human serum albumin (HSA), fusion to polyethylene glycol (PEG), or fusion to an anti-HSA nanobody.

### DETAILED DESCRIPTION

The present disclosure generally relates to variants of IL-12 that possess sub-maximal signaling efficacy through its receptors relative to wild-type IL-12. In one aspect, the disclosure provided herein relates to one or more IL-12 variant polypeptides that bind specifically to IL-12Rβ2 with activity comparable to wild-type IL-12 but has reduced or disrupted binding to IL-12Rβ1 relative to wild-type IL-12. In one aspect, the disclosure relates to one or more IL-12 p40 subunit (IL-12p40) of a variant that dimerizes with IL-12 p35 subunit comparably to wild-type IL-12 but has reduced or disrupted binding to IL-12Rβ1 relative to wild-type IL-12.

In various embodiments, the present disclosure provides a nucleic acid encoding one or more variant of IL-12 that possesses sub-maximal signaling efficacy through its receptors relative to wild-type IL-12. In other embodiments, the disclosure relates to methods of administering a composition comprising one or more variant polypeptides or one or more nucleic acid molecules encoding one or more variants of IL-12 that possesses sub-maximal signaling efficacy through its receptors relative to wild-type IL-12. In some embodiments, the disclosure relates to methods of treating or preventing one or more diseases or disorders by administering a composition comprising one or more variant(s) of IL-12 or one or more nucleic acid molecules encoding one or more variant(s) of IL-12 that possesses sub-maximal signaling efficacy through its receptors relative to wild-type IL-12, alone or in combination with other therapeutic agents.

### Definitions

Unless defined otherwise, all technical and scientific terms used herein have the same meaning as commonly understood by one of ordinary skill in the art to which this disclosure belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, +1%, or ±0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present disclosure may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and a humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

The terms “co-administration”, “co-administer”, and “in combination with” include the administration of two or more therapeutic agents (e.g., a IL-12 variant polypeptide in combination with an additional agent) either simultaneously, concurrently or sequentially within no specific time limits. The term “co-administration” as used herein is meant to encompass conjugated compounds, as well as unconjugated compounds.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous”, “identical,” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

By the term “modulating,” as used herein, is meant mediating a detectable increase or decrease in the activity and/or level of a mRNA, polypeptide, or a response in a subject compared with the activity and/or level of a mRNA, polypeptide or a response in the subject in the absence of a treatment or compound, and/or compared with the activity and/or level of a mRNA, polypeptide, or a response in an otherwise identical but untreated subject. The term encompasses activating, inhibiting and/or otherwise affecting a native signal or response thereby mediating a beneficial therapeutic, prophylactic, or other desired response in a subject, for example, a human.

The terms “multispecific” or “bispecific” are commonly used when referring to agents (e.g., ligands or antibodies) that recognize two or more different antigens by virtue of possessing at least one region (e.g., a ligand or a Fab of a first antibody) that is specific for a first target, and at least a second region (e.g., a ligand or a Fab of a second antibody) that is specific for a second target. A bispecific agent specifically binds to two targets and is thus one type of multispecific agent.

A “mutation,” “mutant,” or “variant,” as used herein, refers to a change in nucleic acid or polypeptide sequence relative to a reference sequence (which may be a naturally-occurring normal or the “wild-type” sequence), and includes translocations, deletions, insertions, and substitutions/point mutations. A “mutant” or “variant” as used herein, refers to either a nucleic acid or protein comprising a mutation.

A “nucleic acid” or “nucleic acid molecule” refers to a polynucleotide and includes poly-ribonucleotides and poly-deoxyribonucleotides. Nucleic acids according to the present disclosure may include any polymer or oligomer of pyrimidine and purine bases, such as cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present disclosure contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, at least 8, at least 15 or at least 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present disclosure may be a peptide nucleic acid (PNA). (See U.S. Pat. No. 6,156,501 which is hereby incorporated by reference in its entirety.) The disclosure also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The terms “patient,” “subject,” “individual,” and the like are used interchangeably herein, and refer to any animal, or cells thereof whether in vivo, in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, mutant polypeptides, variant polypeptides, or a combination thereof.

As used herein, the term “pharmaceutically-acceptable carrier” means a chemical composition with which a composition of the present disclosure may be combined and which, following the combination, can be used to administer the appropriate composition to a subject.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to exhibit non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

To “prevent” a disease or disorder as the term is used herein, means to reduce the severity or frequency of at least one sign or symptom of a disease or disorder that is to be experienced by a subject.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, the terms “therapy” or “therapeutic regimen” refer to those activities taken to prevent, treat or alter a disease or disorder, e.g., a course of treatment intended to reduce or eliminate at least one sign or symptom of a disease or disorder using pharmacological, surgical, dietary and/or other techniques. A therapeutic regimen may include a prescribed dosage of one or more compounds or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound or composition that will elicit the biological, physiologic, clinical or medical response of a cell, tissue, organ, system, or subject that is being sought by the researcher, veterinarian, medical doctor or other clinician. The term “therapeutically effective amount” includes that amount of a compound or composition that, when administered, is sufficient to prevent development of, or treat to some extent, one or more of the signs or symptoms of the disorder or disease being treated. The therapeutically effective amount will vary depending on the compound or composition, the disease and its severity and the age, weight, etc., of the subject to be treated.

To “treat” a disease or disorder as the term is used herein, means to reduce the frequency or severity of at least one sign or symptom of a disease or disorder experienced by a subject. The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), e.g., slowing or arresting their development (e.g., halting the growth of tumors, slowing the rate of tumor growth, halting the rate of cancer cell proliferation, and the like); or (c) relieving the disease symptom(s), i.e., causing regression of the disease and/or symptom(s) (e.g., causing decrease in tumor size, reducing the number of cancer cells present, and the like). Those in need of treatment include those already inflicted (e.g., those with cancer, those with an infection, those with a metabolic disorder, those with macular degeneration, etc.) as well as those in which prevention is desired (e.g., those with increased susceptibility to cancer, those with an increased likelihood of infection, those suspected of having cancer, those suspected of harboring an infection, those with increased susceptibility for metabolic disease, those with increased susceptibility for macular degeneration, etc.).

As used herein, the term “wild-type” refers to a gene or gene product isolated from a naturally occurring source. A wild-type gene is that which is most frequently observed in a population and is thus arbitrarily designated the “normal” or “wild-type” form of the gene. In contrast, the term “modified,” “variant,” or “mutant” refers to a gene or gene product that possesses modifications in sequence and/or functional properties (i.e., altered characteristics) when compared to the wild-type gene or gene product.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

By the term “specifically binds,” as used herein with respect to an IL-12 variant polypeptide, is meant an IL-12 variant polypeptide that recognizes and binds to a specific receptor, such as IL-12Rβ2 or IL-12Rβ1. In some instances, the IL-12 variant polypeptide substantially reduced binding to IL-12Rβ1 relative to wild-type IL-12. For example, an IL-12 variant polypeptide that specifically binds to a receptor from one species may also bind to that receptor from one or more species. However, such cross-species reactivity does not itself alter the classification of an IL-12 variant polypeptide as specific. In another example, an IL-12 variant polypeptide that specifically binds to a receptor may also bind to different allelic forms of the receptor. However, such cross reactivity does not itself alter the classification of an IL-12 variant polypeptide as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an IL-12 variant polypeptide recognizes and binds to a specific protein structure rather than to proteins generally.

### Compositions

In some embodiments, the compositions of the present disclosure comprise one or more IL-12 variant polypeptide molecules, wherein the variant enhances immune responses but has a relatively low toxicity and a relatively wide therapeutic index. In some embodiments, the composition comprises a partial inducer of IL-12 activity by reducing the recruitment of IL-12Rβ1 to the active receptor signaling complex consisting of both IL-12Rβ2 and IL-12Rβ1. In some embodiments, the composition comprises one or more molecules that are able to bind IL-12Rβ2 but have reduced or abolished binding to through IL-12Rβ1. In some embodiments, the composition comprises one or more IL-12 variant polypeptides. In some embodiments, the composition comprises one or more nucleic acid molecules encoding one or more IL-12 variant polypeptides.

### Polypeptides

In some embodiments, the present disclosure comprises one or more IL-12 variant polypeptides, or a fragment thereof, that specifically bind to IL-12Rβ2 but have reduced binding to IL-12Rβ1. In some embodiments, the one or more IL-12 variant polypeptides exhibit increased binding affinity to IL-12Rβ2 as compared to wild-type (WT) IL-12. In some embodiments, the one or more IL-12 variant polypeptides exhibit similar binding affinity to IL-12Rβ2 as compared to WT IL-12. In some embodiments, the one or more IL-12 variant polypeptides exhibit decreased binding affinity to IL-12Rβ1 as compared to WT IL-12.

In some embodiments, the one or more IL-12 variant polypeptides are useful for the treatment or prevention of a disease or disorder. In some embodiments, the disease of disorder is cancer. In some embodiments, the one or more IL-12 variant polypeptides are useful for the treatment or prevention of a disease or disorder, alone or in combination with one or more additional therapeutic agent. In some embodiments, the additional therapeutic agent is a cancer immunotherapeutic agent. In some embodiments, the additional therapeutic agent is a chemotherapeutic agent. In any such embodiments, the disease or disorder may comprise a cancer, such as an acute myeloma leukemia, an anaplastic lymphoma, an astrocytoma, a B-cell cancer, a breast cancer, a colon cancer, an ependymoma, an esophageal cancer, a glioblastoma, a glioma, a leiomyosarcoma, a liposarcoma, a liver cancer, a lung cancer, a mantle cell lymphoma, a melanoma, a neuroblastoma, a non-small cell lung cancer, an oligodendroglioma, an ovarian cancer, a pancreatic cancer, a peripheral T-cell lymphoma, a renal cancer, a sarcoma, a stomach cancer, a carcinoma, a mesothelioma, or a sarcoma.

In some embodiments, the one or more IL-12 variant polypeptides bind to IL-12Rβ2 and exhibit substantially reduced binding to IL-12Rβ1. In some embodiments, the IL-12 variant polypeptides bind to IL-12Rβ1 with a binding affinity that is about 0.000000000001% to about 95% of the binding affinity of wild-type IL-12 to IL-12Rβ1. In some embodiments, the IL-12 variant polypeptide binds to IL-12Rβ1 with a binding affinity that is about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1% or about 0% of the binding affinity of wild-type IL-12 to IL-12Rβ1.

In some embodiments, the one or more IL-12 variant polypeptides bind to IL-12Rβ1 with dissociation constant (K_{D}; higher indicates a lower binding affinity) that is substantially higher than the K_{D} of the IL-12 variant polypeptides and IL-12Rβ2. In some embodiments, the IL-12 variant polypeptide binds to IL-12Rβ2 with a K_{D} that is substantially the same as or lower than the K_{D} of WT IL-12 and IL-12Rβ2.

In some embodiments, the one or more IL-12 variant polypeptides binds to IL-12Rβ1 with a K_{D} that is substantially higher than the K_{D} of WT IL-12 and IL-12Rβ1. In some embodiments, the IL-12 variant polypeptide binds to IL-12Rβ1 with a K_{D} that is at least 10-fold, at least 100-fold, at least 1,000-fold, at least 10,000-fold, at least 100,000-fold, at least 1,000,000-fold greater at least 10,000,000-fold greater, or at least 100,000,000-fold greater than the K_{D} of WT IL-12 and IL-12Rβ1. In some embodiments, the IL-12 variant polypeptide binds to IL-12Rβ1 with a K_{D} of 10 nM or greater, 15 nM or greater, 20 nM or greater, 25 nM or greater, 30 nM or greater, 35 nM or greater, 40 nM or greater, 45 nM or greater, 50 nM or greater, 55 nM or greater, 60 nM or greater, 65 nM or greater, 70 nM or greater, 75 nM or greater, 80 nM or greater, 85 nM or greater, 90 nM or greater, 95 nM or greater, 100 nM or greater, 200 nM or greater, 300 nM or greater, 400 nM or greater, 500 nM or greater, or 1 μM or greater.

In some embodiments, the one or more IL-12 variant polypeptides exhibits substantially reduced agonism towards IL-12Rβ1 and IL-12Rβ2. In some embodiments, the IL-12 variant polypeptide provides agonism towards IL-12Rβ1 and IL-12Rβ2 that is about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1% or about 0% of the maximum agonism of WT IL-12 to IL-12Rβ1 and IL-12Rβ2.

In some embodiments, the one or more IL-12 variant polypeptides require a substantially higher effective concentration to reach 50% of maximal agonism (EC₅₀) of IL-12Rβ1 and IL-12Rβ2. In some embodiments, the IL-12 variant has an EC₅₀ for IL-12Rβ1 and IL-12Rβ2 that is about 20%, about 30%, about 40%, about 50%, about 60%, about 70%, about 80%, about 90%, about 100%, about 110%, about 120%, about 30%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 550%, about 600%, about 650%, about 700%, about 750%, about 800%, about 850%, about 900%, about 950%, about 1000%, about 1100%, about 1200%, about 1300%, about 1400%, about 1500%, about 1600%, about 1700%, about 1800%, about 1900%, about 2000%, about 2100%, or about 2200% of the EC₅₀ of wild-type IL-12 for IL-12Rβ1 and IL-12Rβ2. In some embodiments, the IL-12 variant has an EC₅₀ for IL-12Rβ1 and IL-12Rβ2 that is at least 2-fold higher, at least 5-fold higher, at least 10-fold higher, at least 50-fold higher, at least 100-fold higher, at least 200-fold higher, at least 500-fold higher, or at least 1000-fold higher than the EC₅₀ of WT IL-12 for IL-12Rβ1 and IL-12Rβ2.

In various embodiments, the one or more IL-12 variant polypeptides comprise one or more mutations relative to WT IL-12 polypeptide. In some embodiments, the WT IL-12 polypeptide comprises human WT IL-12. In some embodiments, both the one or more IL-12 variant polypeptide and the WT IL-12 polypeptide comprise a p35 subunit (IL-12p35) and a p40 subunit (IL-12p40). In some embodiments, the IL-12p40 of WT IL-12 comprises the amino acid sequence of SEQ ID NO: 1 (see Table 1 in Example 1 below for sequences). In some embodiments, the IL-12p35 of WT IL-12 comprises the amino acid sequence of SEQ ID NO: 2.

In one embodiment, the IL-12p35 of the one or more IL-12 variant polypeptides comprise the amino acid sequence of WT IL-12p35 with or without a signal peptide. In one embodiment, the IL-12p35 of the one or more IL-12 variant polypeptides comprises the amino acid sequence of SEQ ID NO: 2 or SEQ ID NO: 35. In some embodiments, the IL-12p35 of the one or more IL-12 variant polypeptides further comprises a purification tag. In some embodiments, the purification tag is a polyhistidine tag. In some embodiments, the polyhistidine tag comprises at least 2, at least 3, at least 4, at least 5, at least 6, at least 7, at least 8, at least 9 or at least 10 histidine residues. In one embodiment, the IL-12p35 of the one or more IL-12 variant polypeptide further comprising a purification tag comprises an amino acid sequence of SEQ ID NO: 30. Unless otherwise specified, the term “X” is used below to represent any amino acid.

In one embodiment, the IL-12p40 of the one or more IL-12 variant polypeptides comprise the amino acid sequence of WT IL-12p40 with or without a signal peptide. In one embodiment, the IL-12p40 of the one or more IL-12 variant polypeptides comprises the amino acid sequence of SEQ ID NO: 1 or SEQ ID NO: 34. In some embodiments, the IL-12p40 of the one or more IL-12 variant polypeptides comprises at least one, at least two, or at least three mutations relative to WT IL-12p40 of SEQ ID NO: 1 or SEQ ID NO: 34. In one embodiment, the IL-12p40 of the one or more IL-12 variant polypeptide comprises at least one mutation selected from the group consisting of: H216X, K217X, and K219X, relative to SEQ ID NO: 1. In one embodiment, the IL-12p40 of the one or more IL-12 variant polypeptide comprises at least one mutation selected from the group consisting of: H216A, K217A, and K219A, relative to SEQ ID NO: 1. In one embodiment, the IL-12p40 of the IL-12 variant polypeptide comprises an amino acid sequence selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, with or without a signal peptide. In one embodiment, the IL-12p40 of the IL-12 variant polypeptide comprises an amino acid sequence selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In some embodiments, the one or more IL-12 variant polypeptide, or fragment thereof, comprises an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12. In some embodiments, the one or more IL-12 variant polypeptide, or fragment thereof, comprises an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12; and (ii) including at least one, at least two, or at least three mutations relative to WT IL-12.

In some embodiments, the IL-12p35 of the one or more IL-12 variant polypeptides, or a fragment thereof, comprise an amino acid sequence having 100% sequence identity to WT IL-12p35. In some embodiments, the IL-12p35 of the one or more IL-12 variant polypeptides, or a fragment thereof, comprises an amino acid sequence (i) having 100% sequence identity to WT IL-12p35; and (ii) including no mutations relative to WT IL-12p35.

In some embodiments, the IL-12p40 of the one or more IL-12 variant polypeptide, or a fragment thereof, comprises an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12p40. In some embodiments, the IL-12p40 of the one or more IL-12 variant polypeptide, or a fragment thereof, comprises an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12p40; and (ii) including at least one, at least two, or at least three mutations relative to WT IL-12p40.

In some embodiments, the one or more IL-12 variant polypeptides, or fragment thereof, comprise (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12p40 and including at least one, at least two, or at least three mutations relative to WT IL-12p40; and (ii) an IL-12p35 comprising an amino acid sequence having 100% sequence identity to WT IL-12p35.

In some embodiments, the IL-12p35 of the one or more IL-12 variant polypeptides, or a fragment thereof, comprises an amino acid sequence having 100% sequence identity to SEQ ID NO: 2 or SEQ ID NO: 35. In some embodiments, the IL-12p35 of the one or more IL-12 variant polypeptides, or a fragment thereof, comprises an amino acid sequence (i) having 100% sequence identity to SEQ ID NO: 2 or SEQ ID NO: 35; and (ii) including no mutations relative to SEQ ID NO: 2 or SEQ ID NO: 35.

In some embodiments, the IL-12p40 of the one or more IL-12 variant polypeptides, or a fragment thereof, comprise an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1. In some embodiments, the IL-12p40 of the one or more IL-12 variant polypeptides, or a fragment thereof, comprise an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1; and (ii) including at least one, at least two, or at least three mutations relative to SEQ ID NO: 1.

In some embodiments, the IL-12p40 of the one or more IL-12 variant polypeptides, or a fragment thereof, comprise an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34. In some embodiments, the IL-12p40 of the one or more IL-12 variant polypeptides, or a fragment thereof, comprises an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34; and (ii) including at least one, at least two, or at least three mutations relative to SEQ ID NO: 34.

In some embodiments, the one or more IL-12 variant polypeptides, or fragment thereof, comprise (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1 and including at least one, at least two, or at least three mutations relative to SEQ ID NO: 1; and (ii) an IL-12p35 comprising an amino acid sequence having 100% sequence identity to SEQ ID NO: 2 or SEQ ID NO: 35.

In some embodiments, the one or more IL-12 variant polypeptides, or fragment thereof, comprise (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34 and including at least one, at least two, or at least three mutations relative to SEQ ID NO: 34; and (ii) an IL-12p35 comprising an amino acid sequence having 100% sequence identity to SEQ ID NO: 2 or SEQ ID NO: 35.

In some embodiments, the IL-12p40 of the one or more IL-12 variant polypeptides, or a fragment thereof, comprises an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1; and (ii) including at least one mutation selected from the group consisting of: H216X, K217X, and K219X, relative to SEQ ID NO: 1.

In some embodiments, the IL-12p40 of the one or more IL-12 variant polypeptides, or a fragment thereof, comprise an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34; and (ii) including at least one mutation selected from the group consisting of H216X, K217X, and K219X, relative to SEQ ID NO: 1.

In some embodiments, the one or more IL-12 variant polypeptides, or fragment thereof, comprise (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1 and including at least one mutation selected from the group consisting of: H216X, K217X, and K219X, relative to SEQ ID NO: 1; and (ii) an IL-12p35 comprising an amino acid sequence having 100% sequence identity to SEQ ID NO: 2 or SEQ ID NO: 35.

In some embodiments, the one or more IL-12 variant polypeptides, or fragment thereof, comprise (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34 and including at least one mutation selected from the group consisting of: H216X, K217X, and K219X, relative to SEQ ID NO: 1; and (ii) an IL-12p35 comprising an amino acid sequence having 100% sequence identity to SEQ ID NO: 2 or SEQ ID NO: 35.

In some embodiments, the IL-12p40 of the one or more IL-12 variant polypeptides, or a fragment thereof, comprise an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1; and (ii) including at least one mutation selected from the group consisting of H216A, K217A, and K219A, relative to SEQ ID NO: 1.

In some embodiments, the IL-12p40 of the one or more IL-12 variant polypeptides, or a fragment thereof, comprises an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34; and (ii) including at least one mutation selected from the group consisting of H216A, K217A, and K219A, relative to SEQ ID NO: 1.

In some embodiments, the one or more IL-12 variant polypeptides, or fragment thereof, comprises (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1 and including at least one mutation selected from the group consisting of: H216A, K217A, and K219A, relative to SEQ ID NO: 1; and (ii) an IL-12p35 comprising an amino acid sequence having 100% sequence identity to SEQ ID NO: 2 or SEQ ID NO: 35.

In some embodiments, the one or more IL-12 variant polypeptides, or fragment thereof, comprises (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34 and including at least one mutation selected from the group consisting of: H216A, K217A, and K219A, relative to SEQ ID NO: 1; and (ii) an IL-12p35 comprising an amino acid sequence having 100% sequence identity to SEQ ID NO: 2 or SEQ ID NO: 35.

In some embodiments, the IL-12p40 of the one or more IL-12 variant polypeptides, or a fragment thereof, comprise an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, with or without a signal peptide.

In some embodiments, the IL-12p40 of the one or more IL-12 variant polypeptides, or a fragment thereof, comprise an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In some embodiments, the one or more IL-12 variant polypeptides, or fragment thereof, comprises (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, with or without a signal peptide, and (ii) an IL-12p35 comprising an amino acid sequence having 100% sequence identity to SEQ ID NO: 2 or SEQ ID NO: 35.

In some embodiments, the one or more IL-12 variant polypeptides, or fragment thereof, comprise (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide selected from the group consisting of: SEQ ID NO: 32 and SEQ ID NO: 33, and (ii) an IL-12p35 comprising an amino acid sequence having 100% sequence identity to SEQ ID NO: 2 or SEQ ID NO: 35.

In some cases, it may be desirable to extend the in vivo half-life of the one or more IL-12 variant polypeptide of the present disclosure as described above. Various techniques to extend the in vivo half-life of polypeptides are known in the art including fusing the polypeptide to one or more stable protein or protein domain. Exemplary stabilizing fusion proteins/domains include, but are not limited to, an IgG Fc domain (i.e. capable of forming a homodimeric IgG Fc), an IgG Fc variant domain (i.e. capable of forming a heterodimeric IgG Fc), human serum albumin (HSA), polyethylene glycol (PEG), and an anti-HSA nanobody.

In one embodiment, the IL-12 variant polypeptide(s), as described herein, are fused to a peptide that enhances stability or half-life of the fusion protein. In one embodiment, the fusion peptide comprises at least one region of an immunoglobulin, or a variant or fragment thereof. In one embodiment, the peptide comprises an Fc domain of an immunoglobulin. In one embodiment, the fusion peptide comprises the Fc domain of human IgG1. In one embodiment, the fusion peptide comprises an Fc domain of an immunoglobulin that comprises one or more mutations to remove Fc effector function through Fc receptors or complement. In one embodiment, the fusion peptide comprises an Fc domain of human IgG1 comprising a mutation at residue N297, relative to wildtype human IgG1, rendering the Fc domain aglycosylated.

Heterodimeric Fc fusion constructs are based on the self-assembling nature of the two Fc domains of the heavy chains of antibodies, e.g., two “monomers” that assemble into a “dimer”. Heterodimeric Fc fusions are made by altering the amino acid sequence of each monomer as described in WO2018071919A1, incorporated herein by reference in its entirety. The generation of heterodimeric Fc relies on amino acid variants in the constant regions that are different on each chain to promote heterodimeric formation and/or allow for ease of purification of heterodimers over the homodimers. Thus, in some embodiments, the present disclosure is directed to compositions and methods of using IL-12 variant polypeptide(s), as described herein, fused to heterodimeric Fc, thereby prolonging the half-life of the IL-12 variant polypeptide(s).

In one embodiment, the one or more IL-12 variant polypeptides comprise a bivalent homodimeric Fc. In one embodiment, the bivalent homodimeric Fc comprises at least two IgG Fc domains. In one embodiment, the IgG is human IgG. In one embodiment, the human IgG is human IgG1. In one embodiment, the human IgG1 Fc domain comprises an amino acid sequence of SEQ ID NO: 10.

In one embodiment, the IL-12p40 of the bivalent homodimeric Fc comprises IL-12p40 of WT IL-12. In one embodiment, the IL-12p40 of the bivalent homodimeric Fc comprises the amino acid sequence of SEQ ID NO: 1 or SEQ ID NO: 34. In one embodiment, the IL-12p40 of the bivalent homodimeric Fc comprises at least one, at least two, or at least three mutations relative to WT IL-12p40 of SEQ ID NO: 1 or SEQ ID NO: 34. In one embodiment, the IL-12p40 of the bivalent homodimeric Fc comprises at least one mutation selected from the group consisting of: H216X, K217X, and K219X, relative to SEQ ID NO: 1. In one embodiment, the IL-12p40 of the bivalent homodimeric Fc comprises at least one mutation selected from the group consisting of: H216A, K217A, and K219A, relative to SEQ ID NO: 1.

In one embodiment, the IL-12p35 of the bivalent homodimeric Fc comprises IL-12p35 of WT IL-12. In one embodiment, the IL-12p35 of the bivalent homodimeric Fc comprises a purification tag. In one embodiment, the IL-12p35 of the bivalent homodimeric Fc comprises the amino acid sequence of SEQ ID NO: 2, SEQ ID NO: 30, or SEQ ID NO: 35.

In one embodiment, the IL-12p40 of the bivalent homodimeric Fc is fused to the IgG Fc domain via a linker. In one embodiment, the IL-12p35 of the bivalent homodimeric Fc is fused to the IgG Fc domain via a linker. In one embodiment, the linker comprises an amino acid sequence of SEQ ID NO: 11.

In one embodiment, the IL-12p40 of the bivalent homodimeric Fc fused to the IgG Fc domain via a linker comprises an amino acid sequence of SEQ ID NO: 12. In one embodiment, the IL-12p40 of the bivalent homodimeric Fc fused to the IgG Fc domain via a linker comprises an amino acid sequence of SEQ ID NO: 12, but having one or more mutations in the IL-12p40 portion. In one embodiment, the IL-12p40 of the bivalent homodimeric Fc fused to the IgG Fc domain via a linker comprises an amino acid sequence of SEQ ID NO: 12, but having at least one mutation selected from the group consisting of: H216X, K217X, and K219X, relative to SEQ ID NO: 1 in the IL-12p40 portion. In one embodiment, the IL-12p40 of the bivalent homodimeric Fc fused to the IgG Fc domain via a linker comprises an amino acid sequence of SEQ ID NO: 12, but having at least one mutation selected from the group consisting of: H216A, K217A, and K219A, relative to SEQ ID NO: 1 in the IL-12p40 portion. In one embodiment, the IL-12p35 of the bivalent homodimeric Fc fused to the IgG Fc domain via a linker comprises an amino acid sequence of SEQ ID NO: 13.

In one embodiment, the one or more IL-12 variant polypeptide comprising bivalent homodimeric Fc comprises (i) the IL-12p40 fused to an IgG Fc domain via a linker and (ii) the IL-12p35 of the bivalent homodimeric Fc. In one embodiment, the one or more IL-12 variant polypeptide comprising bivalent homodimeric Fc comprises (i) the IL-12p35 fused to an IgG Fc domain via a linker and (ii) the IL-12p40 of the bivalent homodimeric Fc

In one embodiment, the one or more IL-12 variant polypeptides comprising bivalent homodimeric Fc comprise (i) the IL-12p40 fused to an IgG Fc domain via a linker comprising an amino acid sequence of SEQ ID NO: 12 and (ii) the IL-12p35 comprising an amino acid sequence selected from the group consisting of: SEQ ID NO: 2, SEQ ID NO: 30, and SEQ ID NO: 35. In one embodiment, the one or more IL-12 variant polypeptides comprising bivalent homodimeric Fc comprise (i) the IL-12p40 fused to an IgG Fc domain via a linker comprising an amino acid sequence of SEQ ID NO: 12, but having one or more mutations in the IL-12p40 portion, and (ii) the IL-12p35 comprising an amino acid sequence selected from the group consisting of: SEQ ID NO: 2, SEQ ID NO: 30, and SEQ ID NO: 35. In one embodiment, the one or more IL-12 variant polypeptides comprising bivalent homodimeric Fc comprise (i) the IL-12p40 fused to an IgG Fc domain via a linker comprising an amino acid sequence of SEQ ID NO: 12, but having at least one mutation selected from the group consisting of: H216X, K217X, and K219X, relative to SEQ ID NO: 1 in the IL-12p40 portion, and (ii) the IL-12p35 comprising an amino acid sequence selected from the group consisting of: SEQ ID NO: 2, SEQ ID NO: 30, and SEQ ID NO: 35. In one embodiment, the one or more IL-12 variant polypeptides comprising bivalent homodimeric Fc comprise (i) the IL-12p40 fused to an IgG Fc domain via a linker comprising an amino acid sequence of SEQ ID NO: 12, but having at least one mutation selected from the group consisting of: H216A, K217A, and K219A, relative to SEQ ID NO: 1 in the IL-12p40 portion, and (ii) the IL-12p35 comprising an amino acid sequence selected from the group consisting of: SEQ ID NO: 2, SEQ ID NO: 30, and SEQ ID NO: 35.

In one embodiment, the one or more IL-12 variant polypeptides comprise (i) the IL-12p35 fused to an IgG Fc domain via a linker comprising an amino acid sequence of SEQ ID NO: 13 and (ii) the IL-12p40 of the bivalent homodimeric Fc comprising one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the IL-12p40 of the bivalent homodimeric Fc comprises one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

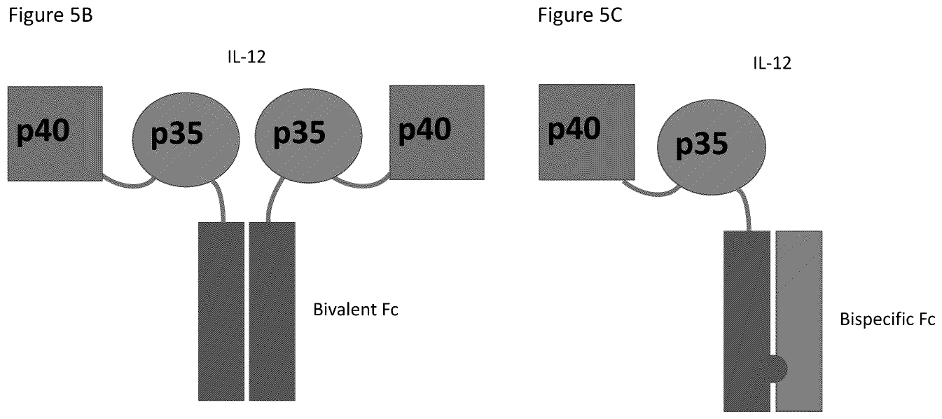

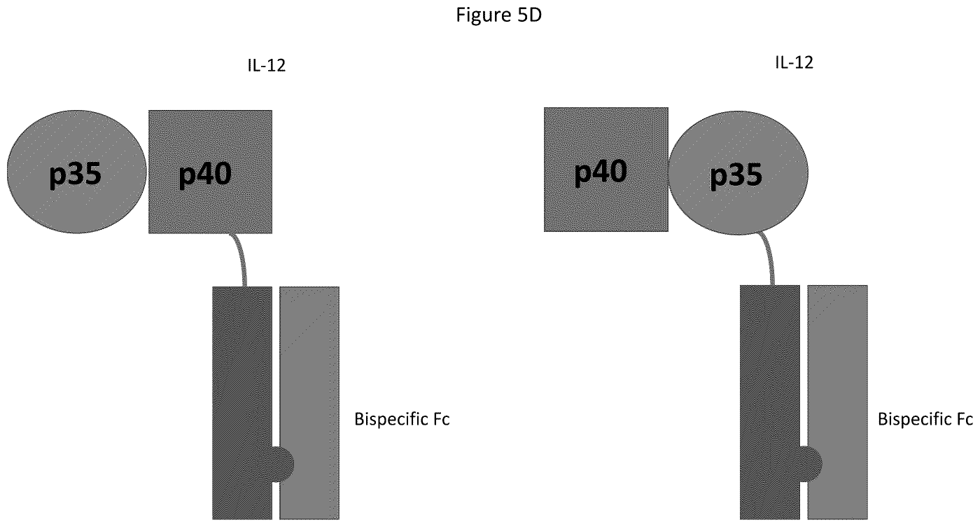

One of skill in the art will recognize that co-expression of either IL-12p40 or IL-12p35 fused to IgG Fc by a linker and the corresponding subunit (IL-12p35 or IL-12p40, respectively) will result in a dimeric IL-12 (i.e. a tetrameric structure comprising a homodimer of heterodimers stabilized by the bivalent IgG Fc domains; see Example 1 and FIG. 5A).

In one embodiment, the one or more IL-12 variant polypeptides comprise a bispecific heterodimeric Fc. In one embodiment, the bispecific heterodimeric Fc comprises an IgG Fc “knob” and an IgG Fc “hole”. In one embodiment, the IgG Fc “knob” and IgG Fc “hole” are variants of IgG Fc. In one embodiment, the IgG Fc comprises human IgG Fc. In one embodiment, the human IgG Fc comprises human IgG1 Fc. In one embodiment, the IgG Fc “knob” comprises an amino acid sequence of SEQ ID NO: 14. In one embodiment, the IgG Fc “hole” comprises an amino acid sequence of SEQ ID NO: 15.

In one embodiment, the IL-12p40 of the bispecific heterodimeric Fc comprises IL-12p40 of WT IL-12. In one embodiment, the IL-12p40 of the bispecific heterodimeric Fc comprises the amino acid sequence of SEQ ID NO: 1 or SEQ ID NO: 34. In one embodiment, the IL-12p40 of the bispecific heterodimeric Fc comprises at least one, at least two, or at least three mutations relative to WT IL-12p40 of SEQ ID NO: 1 or SEQ ID NO: 34. In one embodiment, the IL-12p40 of the bispecific heterodimeric Fc comprises at least one mutation selected from the group consisting of: H216X, K217X, and K219X, relative to SEQ ID NO: 1. In one embodiment, the IL-12p40 of the bispecific heterodimeric Fc comprises at least one mutation selected from the group consisting of: H216A, K217A, and K219A, relative to SEQ ID NO: 1.

In one embodiment, the IL-12p40 of the bispecific heterodimeric Fc comprises one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the IL-12p40 of the bispecific heterodimeric Fc comprises one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In one embodiment, the IL-12p35 of the bispecific heterodimeric Fc comprises IL-12p35 of WT IL-12. In one embodiment, the IL-12p35 of the bispecific heterodimeric Fc comprises a purification tag. In one embodiment, the IL-12p35 of the bispecific heterodimeric Fc comprises the amino acid sequence of SEQ ID NO: 2, SEQ ID NO: 30, or SEQ ID NO: 35.

In one embodiment, the IL-12p40 of the bispecific heterodimeric Fc is fused to the IgG Fc “knob” via a linker. In one embodiment, the IL-12p40 of bispecific heterodimeric Fc is fused to the IgG Fc “hole” via a linker. In one embodiment, the IL-12p35 of bispecific heterodimeric Fc is fused to the IgG Fc “knob” via a linker. In one embodiment, the IL-12p35 of bispecific heterodimeric Fc is fused to the IgG Fc “hole” via a linker. In one embodiment, the linker comprises an amino acid sequence of SEQ ID NO: 11.

In one embodiment, the bispecific heterodimeric Fc comprising the IL-12p40 fused to the IgG Fc “knob” via a linker comprises an amino acid sequence of SEQ ID NO: 16. In one embodiment, the bispecific heterodimeric Fc comprising the IL-12p40 fused to the IgG Fc “knob” via a linker comprises an amino acid sequence of SEQ ID NO: 16, but having one or more mutations in the IL-12p40 portion. In one embodiment, the bispecific heterodimeric Fc comprising the IL-12p40 fused to the IgG Fc “knob” via a linker comprises an amino acid sequence of SEQ ID NO: 16, but having at least one mutation selected from the group consisting of: H216X, K217X, and K219X, relative to SEQ ID NO: 1 in the IL-12p40 portion. In one embodiment, the bispecific heterodimeric Fc comprising the IL-12p40 fused to the IgG Fc “knob” via a linker comprises an amino acid sequence of SEQ ID NO: 16, but having at least one mutation selected from the group consisting of: H216A, K217A, and K219A, relative to SEQ ID NO: 1 in the IL-12p40 portion.

In one embodiment, the bispecific heterodimeric Fc comprising the IL-12p35 fused to the IgG Fc “hole” via a linker comprises an amino acid sequence of SEQ ID NO: 17.

In one embodiment, the bispecific heterodimeric Fc comprising the IL-12p40 fused to the IgG Fc “hole” via a linker comprises an amino acid sequence of SEQ ID NO: 18. In one embodiment, the bispecific heterodimeric Fc comprising the IL-12p40 fused to the IgG Fc “hole” via a linker comprises an amino acid sequence of SEQ ID NO: 18, but having one or more mutations in the IL-12p40 portion. In one embodiment, the bispecific heterodimeric Fc comprising the IL-12p40 fused to the IgG Fc “hole” via a linker comprises an amino acid sequence of SEQ ID NO: 18, but having at least one mutation selected from the group consisting of: H216X, K217X, and K219X, relative to SEQ ID NO: 1 in the IL-12p40 portion. In one embodiment, the bispecific heterodimeric Fc comprising the IL-12p40 fused to the IgG Fc “hole” via a linker comprises an amino acid sequence of SEQ ID NO: 18, but having at least one mutation selected from the group consisting of: H216A, K217A, and K219A, relative to SEQ ID NO: 1 in the IL-12p40 portion.

In one embodiment, the bispecific heterodimeric Fc comprising the IL-12p35 fused to the IgG Fc “knob” via a linker comprises an amino acid sequence of SEQ ID NO: 19.

In one embodiment, the bispecific heterodimeric Fc comprises (i) the IL-12p40 fused to an IgG Fc “knob” via a linker and (ii) the IL-12p35 fused to an IgG Fc “hole” via a linker. In one embodiment, the bispecific heterodimeric Fc comprises (i) the IL-12p35 fused to an IgG Fc “knob” via a linker and (ii) the IL-12p40 fused to an IgG Fc “hole” via a linker.

In one embodiment, the bispecific heterodimeric Fc comprises (i) the IL-12p40 fused to an IgG Fc “knob” via a linker comprising an amino acid sequence of SEQ ID NO: 16 and (ii) the IL-12p35 fused to an IgG Fc “hole” via a linker comprising an amino acid sequence of SEQ ID NO: 17. In one embodiment, the bispecific heterodimeric Fc comprises (i) the IL-12p40 fused to an IgG Fc “knob” via a linker comprising an amino acid sequence of SEQ ID NO: 16, but having one or more mutations in the IL-12p40 portion and (ii) the IL-12p35 fused to an IgG Fc “hole” via a linker comprising an amino acid sequence of SEQ ID NO: 17. In one embodiment, the bispecific heterodimeric Fc comprises (i) the IL-12p40 fused to an IgG Fc “knob” via a linker comprising an amino acid sequence of SEQ ID NO: 16, but having at least one mutation selected from the group consisting of: H216X, K217X, and K219X, relative to SEQ ID NO: 1 in the IL-12p40 portion and (ii) the IL-12p35 fused to an IgG Fc “hole” via a linker comprising an amino acid sequence of SEQ ID NO: 17. In one embodiment, the bispecific heterodimeric Fc comprises (i) the IL-12p40 fused to an IgG Fc “knob” via a linker comprising an amino acid sequence of SEQ ID NO: 16, but having at least one mutation selected from the group consisting of: H216A, K217A, and K219A, relative to SEQ ID NO: 1 in the IL-12p40 portion and (ii) the IL-12p35 fused to an IgG Fc “hole” via a linker comprising an amino acid sequence of SEQ ID NO: 17.

In one embodiment, the bispecific heterodimeric Fc comprises (i) the IL-12p35 fused to an IgG Fc “knob” via a linker comprising an amino acid sequence of SEQ ID NO: 19 and (ii) the IL-12p40 fused to an IgG Fc “hole” via a linker comprising an amino acid sequence of SEQ ID NO: 18. In one embodiment, the bispecific heterodimeric Fc comprises (i) the IL-12p35 fused to an IgG Fc “knob” via a linker comprising an amino acid sequence of SEQ ID NO: 19 and (ii) the IL-12p40 fused to an IgG Fc “hole” via a linker comprising an amino acid sequence of SEQ ID NO: 18, but having one or more mutations in the IL-12p40 portion. In one embodiment, the bispecific heterodimeric Fc comprises (i) the IL-12p35 fused to an IgG Fc “knob” via a linker comprising an amino acid sequence of SEQ ID NO: 19 and (ii) the IL-12p40 fused to an IgG Fc “hole” via a linker comprising an amino acid sequence of SEQ ID NO: 18, but having at least one mutation selected from the group consisting of: H216X, K217X, and K219X. In one embodiment, the bispecific heterodimeric Fc comprises (i) the IL-12p35 fused to an IgG Fc “knob” via a linker comprising an amino acid sequence of SEQ ID NO: 19 and (ii) the IL-12p40 fused to an IgG Fc “hole” via a linker comprising an amino acid sequence of SEQ ID NO: 18, but having at least one mutation selected from the group consisting of: H216A, K217A, and K219A.

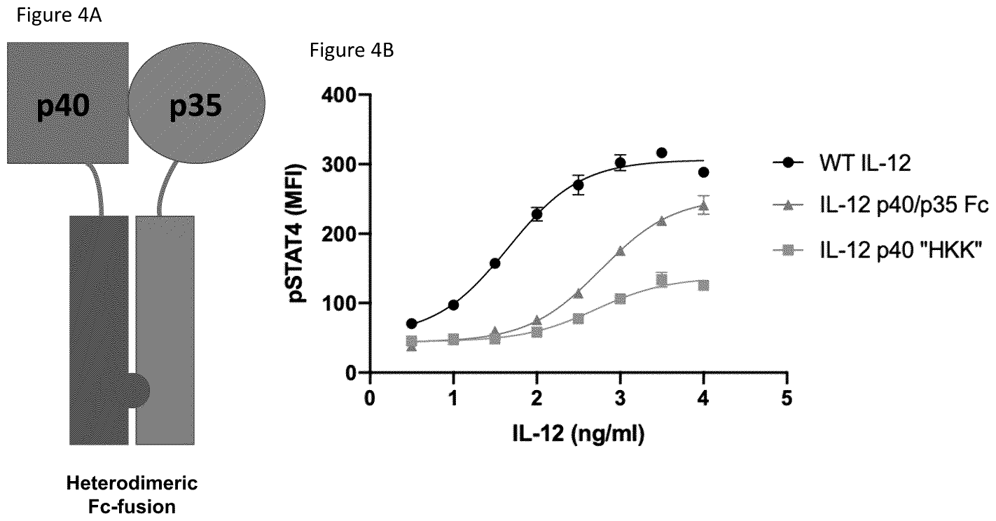

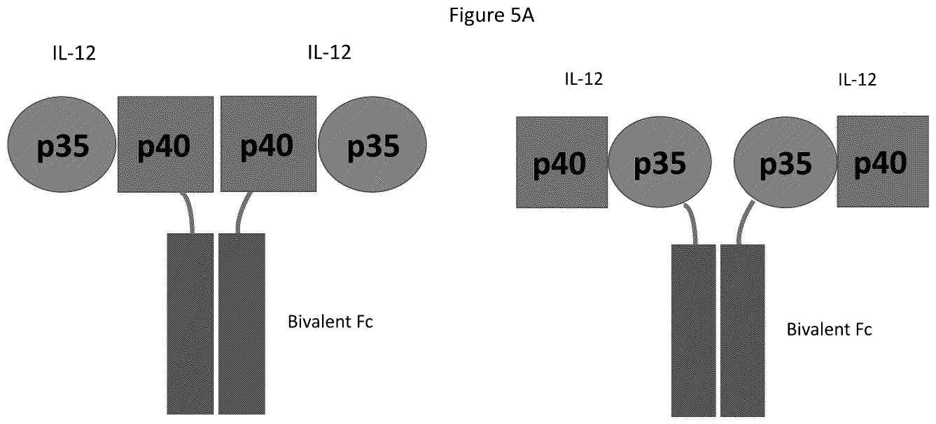

One of skill in the art will recognize that co-expression of either IL-12p40 “knob” or IL-12p35 “hole” with the corresponding subunit (IL-12p40 “hole” or IL-12p35 “knob”, respectively) will result in a modified dimeric IL-12, stabilized by the interaction between the “hole” and “knob” IgG Fc domains (see Example 1, FIG. 4A, and FIG. 5B).

In one embodiment, the one or more IL-12 variant polypeptide comprises a single chain bivalent homodimeric Fc. In one embodiment, the single chain bivalent homodimeric Fc comprises at least two IgG Fc domains. In one embodiment, the IgG is human IgG. In one embodiment, the human IgG is human IgG1. In one embodiment, the human IgG1 Fc domain comprises an amino acid sequence of SEQ ID NO: 10.

In one embodiment, the IL-12p40 of the single chain bivalent homodimeric Fc comprises IL-12p40 of WT IL-12. In one embodiment, the IL-12p40 of the single chain bivalent homodimeric Fc comprises the amino acid sequence of SEQ ID NO: 1 or SEQ ID NO: 34. In one embodiment, the IL-12p40 of the single chain bivalent homodimeric Fc comprises at least one, at least two, or at least three mutations relative to WT IL-12p40 of SEQ ID NO: 1 or SEQ ID NO: 34. In one embodiment, the IL-12p40 of the single chain bivalent homodimeric Fc comprises at least one mutation selected from the group consisting of: H216X, K217X, and K219X, relative to SEQ ID NO: 1. In one embodiment, the IL-12p40 of the single chain bivalent homodimeric Fc comprises at least one mutation selected from the group consisting of: H216A, K217A, and K219A, relative to SEQ ID NO: 1.

In one embodiment, the IL-12p40 of the single chain bivalent homodimeric Fc comprises one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the IL-12p40 of the single chain bivalent homodimeric Fc comprises one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In one embodiment, the IL-12p35 of the single chain bivalent homodimeric Fc comprises IL-12p35 of WT IL-12. In one embodiment, the IL-12p35 of the single chain bivalent homodimeric Fc comprises a purification tag. In one embodiment, the IL-12p35 of the single chain bivalent homodimeric Fc comprises the amino acid sequence of SEQ ID NO: 2, SEQ ID NO: 30, or SEQ ID NO: 35.

In one embodiment, the IL-12p40 of the single chain bivalent homodimeric Fc is fused to the IgG Fc domain via a linker. In one embodiment, the IL-12p35 of the single chain bivalent homodimeric Fc is fused to the IgG Fc domain via a linker. In one embodiment, the IL-12p40 of the single chain bivalent homodimeric Fc is fused to the IL-12p35 of the single chain bivalent homodimeric Fc via a linker.

In one embodiment, (i) the IL-12p40 of the single chain bivalent homodimeric Fc is fused to the IL-12p35 of single chain bivalent homodimeric Fc via a linker and (ii) the IL-12p35 of single chain bivalent homodimeric Fc is fused to the IgG Fc domain via a linker. In one embodiment, (i) the IL-12p40 of single chain bivalent homodimeric Fc is fused to the IL-12p35 of single chain bivalent homodimeric Fc via a linker and (ii) the IL-12p40 of single chain bivalent homodimeric Fc is fused to the IgG Fc domain via a linker. In one embodiment, the linker comprises one or more amino acid sequence selected from the group consisting of: SEQ ID NO: 20 and SEQ ID NO: 21.

In one embodiment, the one or more IL-12 variant polypeptide comprising a single chain bivalent homodimeric Fc comprises the amino acid sequence of SEQ ID NO: 22. In one embodiment, the one or more IL-12 variant polypeptide comprising a single chain bivalent homodimeric Fc comprises the amino acid sequence of SEQ ID NO: 23. In one embodiment, the one or more IL-12 variant polypeptide comprises a single chain bivalent homodimeric Fc comprising the amino acid sequence of SEQ ID NO: 22, but having one more mutations in the IL-12p40 portion of the single chain bivalent homodimeric Fc. In one embodiment, the one or more IL-12 variant polypeptide comprises a single chain bivalent homodimeric Fc comprising the amino acid sequence of SEQ ID NO: 22, but having at least one mutation selected from the group consisting of: H216X, K217X, and K219X, relative to SEQ ID NO: 1 in the IL-12p40 portion of the single chain bivalent homodimeric Fc. In one embodiment, the one or more IL-12 variant polypeptide comprises a single chain bivalent homodimeric Fc comprising the amino acid sequence of SEQ ID NO: 22, but having at least one mutation selected from the group consisting of: H216A, K217A, and K219A, relative to SEQ ID NO: 1 in the IL-12p40 portion of the single chain bivalent homodimeric Fc. In one embodiment, the one or more IL-12 variant polypeptide comprises a single chain bivalent homodimeric Fc comprising the amino acid sequence of SEQ ID NO: 23, but having one more mutations in the IL-12p40 portion of the single chain bivalent homodimeric Fc. In one embodiment, the one or more IL-12 variant polypeptide comprises a single chain bivalent homodimeric Fc comprising the amino acid sequence of SEQ ID NO: 23, but having at least one mutation selected from the group consisting of: H216X, K217X, and K219X, relative to SEQ ID NO: 1 in the IL-12p40 portion of the single chain bivalent homodimeric Fc. In one embodiment, the one or more IL-12 variant polypeptide comprises a single chain bivalent homodimeric Fc comprising the amino acid sequence of SEQ ID NO: 23, but having at least one mutation selected from the group consisting of: H216A, K217A, and K219A, relative to SEQ ID NO: 1 in the IL-12p40 portion of the single chain bivalent homodimeric Fc.

One of skill in the art will recognize that expression of either configuration of single chain bivalent homodimeric Fc will result in a dimeric IL-12 (i.e. a dimer of fused dimers stabilized by the bivalent IgG Fc domains; see Example 1 and FIG. 5B).

In one embodiment, the one or more IL-12 variant polypeptide comprises a single chain monomeric IL-12 and a bispecific heterodimeric Fc.

In one embodiment, the bispecific heterodimeric Fc comprises an IgG Fc “knob” and an IgG Fc “hole”. In one embodiment, the IgG Fc “knob” and IgG Fc “hole” are variants of IgG Fc. In one embodiment, the IgG Fc comprises human IgG Fc. In one embodiment, the human IgG Fc comprises human IgG1 Fc. In one embodiment, the IgG Fc “hole” is fused to signal peptide via a linker. In one embodiment, the IgG Fc “knob” is fused to a signal peptide via a linker. In one embodiment, the linker comprises an amino acid sequence of SEQ ID NO: 11.

In one embodiment, the IL-12p40 of the single chain monomeric IL-12 comprises IL-12p40 of WT IL-12. In one embodiment, the IL-12p40 of the single chain monomeric IL-12 comprises the amino acid sequence of SEQ ID NO: 1 or SEQ ID NO: 34. In one embodiment, the IL-12p40 of the single chain monomeric IL-12 comprises at least one, at least two, or at least three mutations relative to WT IL-12p40 of SEQ ID NO: 1 or SEQ ID NO: 34. In one embodiment, the IL-12p40 of the single chain monomeric IL-12 comprises at least one mutation selected from the group consisting of: H216X, K217X, and K219X, relative to SEQ ID NO: 1. In one embodiment, the IL-12p40 of the single chain monomeric IL-12 comprises at least one mutation selected from the group consisting of: H216A, K217A, and K219A, relative to SEQ ID NO: 1.

In one embodiment, the IL-12p40 of the single chain monomeric IL-12 comprises one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the IL-12p40 of the single chain monomeric IL-12 comprises one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In one embodiment, the IL-12p35 of the single chain monomeric IL-12 comprises IL-12p35 of WT IL-12. In one embodiment, the IL-12p35 of the single chain monomeric IL-12 comprises a purification tag. In one embodiment, the IL-12p35 of the single chain monomeric IL-12 comprises the amino acid sequence of SEQ ID NO: 2, SEQ ID NO: 30, or SEQ ID NO: 35.

In one embodiment, the single chain monomeric IL-12 comprises the IL-12p40 fused via a linker to the IL-12p35. In one embodiment, the single chain monomeric IL-12 comprises (i) the IL-12p40 fused via a linker to the IL-12p35 and (ii) the IL-12p35 fused via a linker to IgG Fc “knob”. In one embodiment, the single chain monomeric IL-12 comprises (i) the IL-12p35 fused via a linker to the IL-12p40 and (ii) the IL-12p40 fused via a linker to IgG Fc “knob”. In one embodiment, the single chain monomeric IL-12 comprises (i) the IL-12p40 fused via a linker to the IL-12p35 and (ii) the IL-12p35 fused via a linker to IgG Fc “hole”. In one embodiment, the single chain monomeric IL-12 comprises (i) the IL-12p35 fused via a linker to the IL-12p40 and (ii) the IL-12p40 fused via a linker to IgG Fc “hole”. In one embodiment, the linker comprises one or more amino acid sequence selected from the group consisting of: SEQ ID NO: 20 and SEQ ID NO: 21.

In one embodiment, the single chain monomeric IL-12 comprises an amino acid sequence of one or more selected from the group consisting of: SEQ ID NO: 26, SEQ ID NO: 27, SEQ ID NO: 28 and SEQ ID NO: 29.

In one embodiment, the single chain monomeric IL-12 comprises an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: of: SEQ ID NO: 26, SEQ ID NO: 27, SEQ ID NO: 28 and SEQ ID NO: 29. In one embodiment, the single chain monomeric IL-12 comprises an amino acid sequence of one or more selected from the group consisting of: SEQ ID NO: 26, SEQ ID NO: 27, SEQ ID NO: 28 and SEQ ID NO: 29, but having one more mutations in the IL-12p40 portion of the single chain monomeric IL-12. In one embodiment, the single chain monomeric IL-12 comprises an amino acid sequence of one or more selected from the group consisting of: SEQ ID NO: 26, SEQ ID NO: 27, SEQ ID NO: 28 and SEQ ID NO: 29, but having at least one mutation selected from the group consisting of: H216X, K217X, and K219X, relative to SEQ ID NO: 1 in the IL-12p40 portion of the single chain monomeric IL-12. In one embodiment, the single chain monomeric IL-12 comprises an amino acid sequence of one or more selected from the group consisting of: SEQ ID NO: 26, SEQ ID NO: 27, SEQ ID NO: 28 and SEQ ID NO: 29, but having at least one mutation selected from the group consisting of: H216A, K217A, and K219A, relative to SEQ ID NO: 1 in the IL-12p40 portion of the single chain monomeric IL-12.

In one embodiment, the one or more IL-12 variant polypeptides comprising a single chain monomeric IL-12 and a bispecific heterodimeric Fc comprise (i) the single chain monomeric IL-12 comprising an amino acid sequence of SEQ ID NO: 26 and (ii) the IgG Fc “hole” comprising an amino acid sequence of SEQ ID NO: 25.

In one embodiment, the one or more IL-12 variant polypeptides comprising a single chain monomeric IL-12 and a bispecific heterodimeric Fc comprise (i) the single chain monomeric IL-12 comprising an amino acid sequence of SEQ ID NO: 27 and (ii) the IgG Fc “hole” comprising an amino acid sequence of SEQ ID NO: 25.

In one embodiment, the one or more IL-12 variant polypeptides comprising a single chain monomeric IL-12 and a bispecific heterodimeric Fc comprise (i) the single chain monomeric IL-12 comprising an amino acid sequence of SEQ ID NO: 28 and (ii) the IgG Fc “knob” comprising an amino acid sequence of SEQ ID NO: 24.

In one embodiment, the one or more IL-12 variant polypeptides comprising a single chain monomeric IL-12 and a bispecific heterodimeric Fc comprise (i) the single chain monomeric IL-12 comprising an amino acid sequence of SEQ ID NO: 27 and (ii) the IgG Fc “knob” comprising an amino acid sequence of SEQ ID NO: 24.

One of skill in the art will recognize the co-expression of an IgG Fc “knob” or “hole” with a single chain IL-12 fused to the corresponding IgG Fc “hole” or “knob” respectively, will result in a monomeric IL-12 stabilized by a heterodimeric Fc (see Example 1 and FIG. 5C).

In one embodiment, the one or more IL-12 variant polypeptide comprises dimeric IL-12 and a bispecific heterodimeric Fc.

In one embodiment, the bispecific heterodimeric Fc comprises an IgG Fc “knob” and an IgG Fc “hole”. In one embodiment, the IgG Fc “knob” and IgG Fc “hole” are variants of IgG Fc. In one embodiment, the IgG Fc comprises human IgG Fc. In one embodiment, the human IgG Fc comprises human IgG1 Fc. In one embodiment, the IgG Fc “hole” is fused to signal peptide via a linker. In one embodiment, the IgG Fc “knob” is fused to a signal peptide via a linker. In one embodiment, the linker comprises an amino acid sequence of SEQ ID NO: 11.

In one embodiment, the IL-12p40 of the dimeric IL-12 comprises IL-12p40 of WT IL-12. In one embodiment, the IL-12p40 of the dimeric IL-12 comprises the amino acid sequence of SEQ ID NO: 1 or SEQ ID NO: 34. In one embodiment, the IL-12p40 of the dimeric IL-12 comprises at least one, at least two, or at least three mutations relative to WT IL-12p40 of SEQ ID NO: 1 or SEQ ID NO: 34. In one embodiment, the IL-12p40 of the dimeric IL-12 comprises at least one mutation selected from the group consisting of: H216X, K217X, and K219X, relative to SEQ ID NO: 1. In one embodiment, the IL-12p40 of the dimeric IL-12 comprises at least one mutation selected from the group consisting of: H216A, K217A, and K219A, relative to SEQ ID NO: 1.

In one embodiment, the IL-12p40 of the dimeric IL-12 comprises one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the IL-12p40 of the dimeric IL-12 comprises one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In one embodiment, the IL-12p35 of the dimeric IL-12 comprises IL-12p35 of WT IL-12. In one embodiment, the IL-12p35 of the dimeric IL-12 comprises a purification tag. In one embodiment, the IL-12p35 of the dimeric IL-12 comprises the amino acid sequence of SEQ ID NO: 2, SEQ ID NO: 30, or SEQ ID NO: 35.

In one embodiment, the dimeric IL-12 comprises (i) the IL-12p40 of the dimeric IL-12 and (ii) the IL-12p35 fused via a linker to IgG Fc “knob”. In one embodiment, the dimeric IL-12 comprises (i) the IL-12p40 of the dimeric IL-12 and (ii) the IL-12p35 fused via a linker to IgG Fc “hole”. In one embodiment, the dimeric IL-12 comprises (i) the IL-12p35 of the dimeric IL-12 and (ii) the IL-12p40 fused via a linker to IgG Fc “knob”. In one embodiment, the dimeric IL-12 comprises (i) the IL-12p35 of the dimeric IL-12 and (ii) the IL-12p40 fused via a linker to IgG Fc “hole”.

In one embodiment, the one or more IL-12 variant polypeptide comprising dimeric IL-12 and a bispecific heterodimeric Fc comprises (i) the IL-12p40 of the dimeric IL-12, (ii) the IL-12p35 fused via a linker to IgG Fc “knob”, and (iii) the IgG Fc “hole”. In one embodiment, the one or more IL-12 variant polypeptide comprising dimeric IL-12 and a bispecific heterodimeric Fc comprises (i) the IL-12p35 of the dimeric IL-12, (ii) the IL-12p40 fused via a linker to IgG Fc “knob”, and (iii) the IgG Fc “hole”. In one embodiment, the one or more IL-12 variant polypeptide comprising dimeric IL-12 and a bispecific heterodimeric Fc comprises (i) the IL-12p40 of the dimeric IL-12, (ii) the IL-12p35 fused via a linker to IgG Fc “hole”, and (iii) the IgG Fc “knob”. In one embodiment, the one or more IL-12 variant polypeptide comprising dimeric IL-12 and a bispecific heterodimeric Fc comprises (i) the IL-12p35 of the dimeric IL-12, (ii) the IL-12p40 fused via a linker to IgG Fc “hole”, and (iii) the IgG Fc “knob”.

In one embodiment, the one or more IL-12 variant polypeptide comprising dimeric IL-12 and a bispecific heterodimeric Fc comprises (i) the IL-12p40 comprising an amino acid sequence of one or more selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34, (ii) the IL-12p35 fused via a linker to IgG Fc “knob” comprising an amino acid sequence of SEQ ID NO: 19, and (iii) the IgG Fc “hole” comprising an amino acid sequence of SEQ ID NO: 25.

In one embodiment, the one or more IL-12 variant polypeptide comprising dimeric IL-12 and a bispecific heterodimeric Fc comprises (i) the IL-12p40 comprising an amino acid sequence of one or more selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide selected from the group consisting of: SEQ ID NO: 32 and SEQ ID NO: 33, (ii) the IL-12p35 fused via a linker to IgG Fc “knob” comprising an amino acid sequence of SEQ ID NO: 19, and (iii) the IgG Fc “hole” comprising an amino acid sequence of SEQ ID NO: 25.

In one embodiment, the one or more IL-12 variant polypeptide comprising dimeric IL-12 and a bispecific heterodimeric Fc comprises (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “knob” comprising an amino acid sequence of SEQ ID NO: 16, and (iii) the IgG Fc “hole” comprising an amino acid sequence of SEQ ID NO: 25. In one embodiment, the one or more IL-12 variant polypeptide comprising dimeric IL-12 and a bispecific heterodimeric Fc comprises (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “knob” comprising an amino acid sequence of SEQ ID NO: 16, but having one or more mutations in the IL-12p40 portion, and (iii) the IgG Fc “hole” comprising an amino acid sequence of SEQ ID NO: 25. In one embodiment, the one or more IL-12 variant polypeptide comprising dimeric IL-12 and a bispecific heterodimeric Fc comprises (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “knob” comprising an amino acid sequence of SEQ ID NO: 16, but having at least one mutation selected from the group consisting of: H216X, K217X, and K219X, relative to SEQ ID NO: 1 in the IL-12p40 portion, and (iii) the IgG Fc “hole” comprising an amino acid sequence of SEQ ID NO: 25. In one embodiment, the one or more IL-12 variant polypeptide comprising dimeric IL-12 and a bispecific heterodimeric Fc comprises (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “knob” comprising an amino acid sequence of SEQ ID NO: 16, but having at least one mutation selected from the group consisting of: H216A, K217A, and K219A, relative to SEQ ID NO: 1 in the IL-12p40 portion, and (iii) the IgG Fc “hole” comprising an amino acid sequence of SEQ ID NO: 25.

In one embodiment, the one or more IL-12 variant polypeptide comprising dimeric IL-12 and a bispecific heterodimeric Fc comprises (i) the IL-12p40 comprising an amino acid sequence of one or more selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34, (ii) the IL-12p35 fused via a linker to IgG Fc “hole” comprising an amino acid sequence of SEQ ID NO: 17, and (iii) the IgG Fc “knob” comprising an amino acid sequence of SEQ ID NO: 24.

In one embodiment, the one or more IL-12 variant polypeptide comprising dimeric IL-12 and a bispecific heterodimeric Fc comprises (i) the IL-12p40 comprising an amino acid sequence of one or more selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide selected from the group consisting of: SEQ ID NO: 32 and SEQ ID NO: 33, (ii) the IL-12p35 fused via a linker to IgG Fc “hole” comprising an amino acid sequence of SEQ ID NO: 17, and (iii) the IgG Fc “knob” comprising an amino acid sequence of SEQ ID NO: 24.

In one embodiment, the one or more IL-12 variant polypeptide comprising dimeric IL-12 and a bispecific heterodimeric Fc comprises (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “hole” comprising an amino acid sequence of SEQ ID NO: 18, and (iii) the IgG Fc “knob” comprising an amino acid sequence of SEQ ID NO: 24. In one embodiment, the one or more IL-12 variant polypeptide comprising dimeric IL-12 and a bispecific heterodimeric Fc comprises (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “hole” comprising an amino acid sequence of SEQ ID NO: 18, but having at least one mutation in the IL-12p40 portion, and (iii) the IgG Fc “knob” comprising an amino acid sequence of SEQ ID NO: 24. In one embodiment, the one or more IL-12 variant polypeptide comprising dimeric IL-12 and a bispecific heterodimeric Fc comprises (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “hole” comprising an amino acid sequence of SEQ ID NO: 18, but having at least one mutation selected from the group consisting of: H216X, K217X, and K219X, relative to SEQ ID NO: 1 in the IL-12p40 portion, and (iii) the IgG Fc “knob” comprising an amino acid sequence of SEQ ID NO: 24. In one embodiment, the one or more IL-12 variant polypeptide comprising dimeric IL-12 and a bispecific heterodimeric Fc comprises (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “hole” comprising an amino acid sequence of SEQ ID NO: 18, but having at least one mutation selected from the group consisting of: H216A, K217A, and K219A, relative to SEQ ID NO: 1 in the IL-12p40 portion, and (iii) the IgG Fc “knob” comprising an amino acid sequence of SEQ ID NO: 24.

One of skill in the art will recognize that co-expression of IL-12p40 or IL-12p35 with (i) the corresponding subunit fused to IgG Fc “hole” or “knob” and (ii) the corresponding IgG Fc “knob” or “hole” will result in a dimeric IL-12 stabilized by heterodimeric IgG Fc (see Example 1 and FIG. 5D).

Human serum albumin (HSA) has been genetically fused to therapeutically beneficial peptides (WO2001079271A and WO2003059934A, incorporated herein by reference in their entireties) with the typical result that the fusion has the activity of the therapeutically beneficial peptide and a considerably longer plasma half-life than the plasma half-life of the therapeutically beneficial peptides alone. Thus, in some embodiments, the present disclosure is directed to compositions and methods of using IL-12 variant polypeptide(s), as described herein, fused to HSA, thereby prolonging the half-life of the IL-12 variant polypeptide(s).

One of the most widely used methods for improving the stability of proteins is the chemical modification of a polypeptide with highly soluble macromolecules such as polyethylene glycol (“PEG”) which prevents the polypeptides from contacting with proteases. PEG is a highly flexible, uncharged, mostly non-immunogenic, hydrophilic, non-biodegradable molecule, which generates a larger hydrodynamic radius than an equivalently sized protein. It is also well known that, when linked to a polypeptide drugs specifically or non-specifically, PEG increases the solubility of the polypeptide drug and prevents the hydrolysis thereof, thereby increasing the serum stability of the polypeptide drug without incurring any immune response due to its low antigenicity (Sada et al, J. Fermentation Bioengineering, 1991, 71: 137-139). Thus, in some embodiments, the present disclosure is directed to compositions and methods of using IL-12 variant polypeptide(s), as described herein, fused to PEG, thereby prolonging the half-life of the IL-12 variant polypeptide(s).

Another method for improving the in vivo half-life of proteins comprises fusion to single domain antibodies as described in WO2004041865, incorporated by reference herein in its entirety. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. According to one aspect of the disclosure, a single domain antibody as used herein is a naturally occurring single domain antibody known as heavy chain antibody devoid of light chains. For clarity reasons, this variable domain derived from a heavy chain antibody naturally devoid of light chain is known herein as a VHH or nanobody to distinguish it from the conventional VH of four chain immunoglobulins. Such a VHH molecule or nanobody can be derived from antibodies raised in Camelidae species, for example in camel, dromedary, alpaca and guanaco. Thus, in some embodiments, the present disclosure is directed to compositions and methods of using IL-12 variant polypeptide(s), as described herein, fused to a nanobody, thereby prolonging the half-life of the IL-12 variant polypeptide(s). In one embodiment, the nanobody comprises an anti-HSA nanobody.

In some embodiments, the one or more IL-12 variant polypeptide further comprises one or more signal peptide. In one embodiment, the one or more signal peptide promotes extracellular secretion of the one or more IL-12 variant polypeptide. In one embodiment, the one or more signal peptide comprises one or more amino acid sequence selected from the group consisting of: SEQ ID NO: 31, SEQ ID NO: 32, and SEQ ID NO: 33.

One of skill in the art will recognize that any known methods of producing polypeptides can be used to generate the polypeptide(s) of the present disclosure. The polypeptide(s) of the present disclosure may be made using chemical methods. For example, polypeptide(s) can be synthesized by solid phase techniques (Roberge J Y et al (1995) Science 269: 202-204), cleaved from the resin, and purified by preparative high-performance liquid chromatography. Automated synthesis may be achieved, for example, using the ABI 431 A Peptide Synthesizer (Perkin Elmer) in accordance with the instructions provided by the manufacturer.

Polypeptide(s) of the disclosure may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the disclosure may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

The polypeptide(s) may alternatively be made by recombinant means or by cleavage from one or more longer polypeptide(s). The composition of the polypeptide(s) may be confirmed by amino acid analysis or sequencing.

The variants of the polypeptide(s) according to the present disclosure may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the polypeptide(s) of the present disclosure, (iv) fragments of the polypeptide(s) and/or (v) one in which the polypeptide(s) is/are fused with another peptide, such as a leader or secretory sequence or a sequence which is employed for purification (for example, His-tag) or for detection (for example, Sv5 epitope tag). The fragments include polypeptide(s) generated via proteolytic cleavage (including multi-site proteolysis) of an original sequence. Variants may be post-translationally, or chemically modified. Such variants are deemed to be within the scope of those skilled in the art from the teaching herein.

The polypeptide(s) of the disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptide(s) of the disclosure may include unnatural amino acids formed by post-translational modification or by introducing unnatural amino acids during translation. A variety of approaches are available for introducing unnatural amino acids during protein translation.

The polypeptide(s) of the disclosure may be phosphorylated using conventional methods such as the method described in Reedijk et al. (The EMBO Journal 11(4):1365, 1992).

The polypeptide(s) of the disclosure may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In some other embodiments, IL-12 variant polypeptides of the disclosure include reagents further modified to improve their resistance to proteolytic degradation or to optimize solubility properties or to render them more suitable as a therapeutic agent. For example, variants of the present disclosure further include analogs containing residues other than naturally occurring L-amino acids, e.g. D-amino acids or non-naturally occurring synthetic amino acids. D-amino acids may be substituted for some or all of the amino acid residues.

In some embodiments, the one or more IL-12 variant polypeptide can be fused to another protein (i.e., a “second polypeptide”). In some embodiments, the second polypeptide specifically binds to a target molecule other than the target molecule bound by the one or more IL-12 variant polypeptide (e.g., other than IL-12Rβ1 and/or IL-12Rβ2). Thus, in some embodiments, a the one or more IL-12 variant polypeptide is multispecific (e.g., bispecific), such that a first region of the polypeptide includes an IL-12 variant polypeptide sequence (i.e., the first region includes an IL-12 variant polypeptide), and a second region that specifically binds to another target molecule (e.g., an antigen). For example, in some cases, an IL-12 variant polypeptide is fused to a second polypeptide that binds specifically to a target molecule other than the target molecule bound by the IL-12 variant polypeptide.

In some embodiments, the one or more IL-12 variant polypeptide includes a linker (e.g., a linker polypeptide). For example, in some embodiments, one or more IL-12 variant polypeptide and a fusion partner (i.e. second polypeptide) are separated by a linker (e.g., a linker polypeptide). A linker polypeptide may have any of a variety of amino acid sequences. Proteins can be joined by a linker polypeptide can be of a flexible nature (e.g., a flexible linker polypeptide), although other chemical linkages are not excluded. Suitable linkers include polypeptides of between about 6 amino acids and about 40 amino acids in length, or between about 6 amino acids and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that the in some case, linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. In some embodiments, the linker comprises one or more amino acid sequence selected from the group consisting of: SEQ ID NO: 11, SEQ ID NO: 20 and SEQ ID NO: 21.

### Nucleic Acids

In some embodiments, the present disclosure comprises one or more nucleic acid molecule encoding one or more IL-12 variant polypeptide of the present disclosure, as described above. In some embodiments, the present disclosure comprises at least two nucleic acid molecules encoding one or more IL-12 variant polypeptide as described above.

In some embodiments, the present disclosure comprises one or more nucleic acid molecule encoding IL-12p40 of the one or more IL-12 variant polypeptide of the present disclosure, as described above. In some embodiments, the present disclosure comprises one or more nucleic acid molecule encoding IL-12p35 of the one or more IL-12 variant polypeptide of the present disclosure, as described above.

In some embodiments, the present disclosure comprises one or more nucleic acid molecule encoding IL-12p40 and IL-12p35 of the one or more IL-12 variant polypeptide of the present disclosure, as described above. In some embodiments, the present disclosure comprises at least two nucleic acid molecules encoding IL-12p40 and IL-12p35 of the one or more IL-12 variant polypeptide as described above.

In one embodiment, the nucleic acid molecule encodes an IL-12p40 of the one or more IL-12 variant polypeptide, wherein the IL-12p40 comprises the amino acid sequence of WT IL-12p40 with or without a signal peptide. In one embodiment, the nucleic acid molecule encodes an IL-12p40 of the one or more IL-12 variant polypeptide, wherein the IL-12p40 comprises the amino acid sequence of SEQ ID NO: 1 or SEQ ID NO: 34. In some embodiments, the nucleic acid molecule encodes an IL-12p40 of the one or more IL-12 variant polypeptide, wherein the IL-12p40 comprises at least one, at least two, or at least three mutations relative to WT IL-12p40 of SEQ ID NO: 1 or SEQ ID NO: 34. In one embodiment, the nucleic acid molecule encodes an IL-12p40 of the one or more IL-12 variant polypeptide, wherein the IL-12p40 comprises at least one mutation selected from the group consisting of: H216X, K217X, and K219X, relative to SEQ ID NO: 1. In one embodiment, the nucleic acid molecule encodes an IL-12p40 of the one or more IL-12 variant polypeptide, wherein the IL-12p40 comprises at least one mutation selected from the group consisting of: H216A, K217A, and K219A, relative to SEQ ID NO: 1. In one embodiment, the nucleic acid molecule encodes an IL-12p40 of the one or more IL-12 variant polypeptide, wherein the IL-12p40 comprises an amino acid sequence selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, with or without a signal peptide. In one embodiment, the nucleic acid molecule encodes an IL-12p40 of the one or more IL-12 variant polypeptide, wherein the IL-12p40 comprises an amino acid sequence selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In some embodiments, the nucleic acid molecule encodes one or more IL-12 variant polypeptide, or fragment thereof, comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12. In some embodiments, the nucleic acid molecule encodes one or more IL-12 variant polypeptide, or fragment thereof, comprising an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12; and (ii) including at least one, at least two, or at least three mutations relative to WT IL-12.

In some embodiments, the nucleic acid molecule encodes an IL-12p35 of the one or more IL-12 variant polypeptide, or a fragment thereof, comprising an amino acid sequence having 100% sequence identity to WT IL-12p35. In some embodiments, the nucleic acid molecule encode an IL-12p35 of the one or more IL-12 variant polypeptide, or a fragment thereof, comprising an amino acid sequence (i) having 100% sequence identity to WT IL-12p35; and (ii) including no mutations relative to WT IL-12p35.

In some embodiments, the nucleic acid molecule encodes an IL-12p40 of the one or more IL-12 variant polypeptide, or a fragment thereof, wherein the IL-12p40 comprises an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12p40. In some embodiments, the nucleic acid molecule encodes an IL-12p40 of the one or more IL-12 variant polypeptide, or a fragment thereof, wherein the IL-12p40 comprises an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12p40; and (ii) including at least one, at least two, or at least three mutations relative to WT IL-12p40.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to WT IL-12p40 and including at least one, at least two, or at least three mutations relative to WT IL-12p40; and (ii) an IL-12p35 comprising an amino acid sequence having 100% sequence identity to WT IL-12p35.

In some embodiments, the nucleic acid molecule encodes an IL-12p35 of the one or more IL-12 variant polypeptide, or a fragment thereof, comprising an amino acid sequence having 100% sequence identity to SEQ ID NO: 2 or SEQ ID NO: 35. In some embodiments, the nucleic acid molecule encodes an IL-12p35 of the one or more IL-12 variant polypeptide, or a fragment thereof, comprising an amino acid sequence (i) having 100% sequence identity to SEQ ID NO: 2 or SEQ ID NO: 35; and (ii) including no mutations relative to SEQ ID NO: 2 or SEQ ID NO: 35.

In some embodiments, the nucleic acid molecule encodes an IL-12p40 of the one or more IL-12 variant polypeptide, or a fragment thereof, comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1. In some embodiments, the nucleic acid molecule encodes an IL-12p40 of the one or more IL-12 variant polypeptide, or a fragment thereof, comprising an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1; and (ii) including at least one, at least two, or at least three mutations relative to SEQ ID NO: 1.

In some embodiments, the nucleic acid molecule encodes an IL-12p40 of the one or more IL-12 variant polypeptide, or a fragment thereof, comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34. In some embodiments, the nucleic acid molecule encodes an IL-12p40 of the one or more IL-12 variant polypeptide, or a fragment thereof, comprising an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34; and (ii) including at least one, at least two, or at least three mutations relative to SEQ ID NO: 34.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1 and including at least one, at least two, or at least three mutations relative to SEQ ID NO: 1; and (ii) an IL-12p35 comprising an amino acid sequence having 100% sequence identity to SEQ ID NO: 2 or SEQ ID NO: 35.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34 and including at least one, at least two, or at least three mutations relative to SEQ ID NO: 34; and (ii) an IL-12p35 comprising an amino acid sequence having 100% sequence identity to SEQ ID NO: 2 or SEQ ID NO: 35.

In some embodiments, the nucleic acid molecule encodes an IL-12p40 of the one or more IL-12 variant polypeptide, or a fragment thereof, comprising an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1; and (ii) including at least one mutation selected from the group consisting of: H216X, K217X, and K219X, relative to SEQ ID NO: 1.

In some embodiments, the nucleic acid molecule encodes an IL-12p40 of the one or more IL-12 variant polypeptide, or a fragment thereof, comprising an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34; and (ii) including at least one mutation selected from the group consisting of: H216X, K217X, and K219X, relative to SEQ ID NO: 1.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1 and including at least one mutation selected from the group consisting of: H216X, K217X, and K219X, relative to SEQ ID NO: 1; and (ii) an IL-12p35 comprising an amino acid sequence having 100% sequence identity to SEQ ID NO: 2 or SEQ ID NO: 35.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34 and including at least one mutation selected from the group consisting of: H216X, K217X, and K219X, relative to SEQ ID NO: 1; and (ii) an IL-12p35 comprising an amino acid sequence having 100% sequence identity to SEQ ID NO: 2 or SEQ ID NO: 35.

In some embodiments, the nucleic acid molecule encodes an IL-12p40 of the one or more IL-12 variant polypeptide, or a fragment thereof, comprising an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1; and (ii) including at least one mutation selected from the group consisting of: H216A, K217A, and K219A, relative to SEQ ID NO: 1.

In some embodiments, the IL-12p40 of the one or more IL-12 variant polypeptide, or a fragment thereof, comprises an amino acid sequence (i) having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34; and (ii) including at least one mutation selected from the group consisting of: H216A, K217A, and K219A, relative to SEQ ID NO: 1.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 1 and including at least one mutation selected from the group consisting of: H216A, K217A, and K219A, relative to SEQ ID NO: 1; and (ii) an IL-12p35 comprising an amino acid sequence having 100% sequence identity to SEQ ID NO: 2 or SEQ ID NO: 35.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to SEQ ID NO: 34 and including at least one mutation selected from the group consisting of: H216A, K217A, and K219A, relative to SEQ ID NO: 1; and (ii) an IL-12p35 comprising an amino acid sequence having 100% sequence identity to SEQ ID NO: 2 or SEQ ID NO: 35.

In some embodiments, the nucleic acid molecule encodes an IL-12p40 of the one or more IL-12 variant polypeptide, or a fragment thereof, comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, with or without a signal peptide.

In some embodiments, the nucleic acid molecule encodes IL-12p40 of the one or more IL-12 variant polypeptide, or a fragment thereof, comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, with or without a signal peptide, and (ii) an IL-12p35 comprising an amino acid sequence having 100% sequence identity to SEQ ID NO: 2 or SEQ ID NO: 35.

In some embodiments, the nucleic acid molecule encodes one or more IL-12 variant polypeptide, or fragment thereof, comprising (i) an IL-12p40 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide selected from the group consisting of: SEQ ID NO: 32 and SEQ ID NO: 33, and (ii) an IL-12p35 comprising an amino acid sequence having 100% sequence identity to SEQ ID NO: 2 or SEQ ID NO: 35.

In one embodiment, the nucleic acid molecule encodes an IL-12 variant polypeptide(s), as described herein, are fused to a peptide that enhances stability or half-life of the fusion protein. In one embodiment, the nucleic acid molecule encodes a fusion peptide comprising at least one region of an immunoglobulin, or a variant or fragment thereof. In one embodiment, the nucleic acid molecule encodes a peptide comprising an Fc domain of an immunoglobulin. In one embodiment, the nucleic acid molecule encodes a fusion peptide comprising the Fc domain of human IgG1. In one embodiment, the nucleic acid molecule encodes a fusion peptide comprising an Fc domain of an immunoglobulin that comprises one or more mutations to remove Fc effector function through Fc receptors or complement. In one embodiment, the nucleic acid molecule encodes a fusion peptide comprising an Fc domain of human IgG1 comprising a mutation at residue N297, relative to wildtype human IgG1, rendering the Fc domain aglycosylated.

In some embodiments, the nucleic acid molecule encodes an IL-12 variant polypeptide(s), as described herein, fused to heterodimeric Fc, thereby prolonging the half-life of the IL-12 variant polypeptide(s).

In one embodiment, the nucleic acid molecule encodes one or more IL-12 variant polypeptide comprising a bivalent homodimeric Fc. In one embodiment, the nucleic acid molecule encodes a bivalent homodimeric Fc comprising at least two IgG Fc domains. In one embodiment, the nucleic acid molecule encodes an IgG, wherein the IgG is human IgG. In one embodiment, the nucleic acid molecule encodes human IgG, wherein the human IgG is human IgG1. In one embodiment, the nucleic acid molecule encodes human IgG1 Fc domain comprising an amino acid sequence of SEQ ID NO: 10.

In one embodiment, the nucleic acid molecule encodes an IL-12p40 of the bivalent homodimeric Fc comprising IL-12p40 of WT IL-12. In one embodiment, the nucleic acid molecule encodes an IL-12p40 of the bivalent homodimeric Fc comprising the amino acid sequence of SEQ ID NO: 1 or SEQ ID NO: 34. In one embodiment, the nucleic acid molecule encodes an IL-12p40 of the bivalent homodimeric Fc comprising at least one, at least two, or at least three mutations relative to WT IL-12p40 of SEQ ID NO: 1 or SEQ ID NO: 34. In one embodiment, the nucleic acid molecule encodes IL-12p40 of the bivalent homodimeric Fc comprising at least one mutation selected from the group consisting of: H216X, K217X, and K219X, relative to SEQ ID NO: 1. In one embodiment, the nucleic acid molecule encodes an IL-12p40 of the bivalent homodimeric Fc comprising at least one mutation selected from the group consisting of: H216A, K217A, and K219A, relative to SEQ ID NO: 1.

In one embodiment, the nucleic acid molecule encodes an IL-12p35 of the bivalent homodimeric Fc comprising IL-12p35 of WT IL-12. In one embodiment, the nucleic acid molecule encodes an IL-12p35 of the bivalent homodimeric Fc comprising a purification tag. In one embodiment, the nucleic acid molecule encodes an IL-12p35 of the bivalent homodimeric Fc comprising the amino acid sequence of SEQ ID NO: 2, SEQ ID NO: 30, or SEQ ID NO: 35.

In one embodiment, the nucleic acid molecule encodes a bivalent homodimeric Fc, wherein IL-12p40 is fused to the IgG Fc domain via a linker. In one embodiment, the nucleic acid molecule encodes a bivalent homodimeric Fc, wherein IL-12p35 is fused to the IgG Fc domain via a linker. In one embodiment, the linker comprises an amino acid sequence of SEQ ID NO: 11.

In one embodiment, the nucleic acid molecule encodes IL-12p40 of the bivalent homodimeric Fc fused to the IgG Fc domain via a linker comprising an amino acid sequence of SEQ ID NO: 12. In one embodiment, the nucleic acid molecule encodes IL-12p40 of the bivalent homodimeric Fc fused to the IgG Fc domain via a linker comprising an amino acid sequence of SEQ ID NO: 12, but having one or more mutations in the IL-12p40 portion. In one embodiment, the nucleic acid molecule encodes IL-12p40 of the bivalent homodimeric Fc fused to the IgG Fc domain via a linker comprising an amino acid sequence of SEQ ID NO: 12, but having at least one mutation selected from the group consisting of: H216X, K217X, and K219X, relative to SEQ ID NO: 1 in the IL-12p40 portion. In one embodiment, the nucleic acid molecule encodes IL-12p40 of the bivalent homodimeric Fc fused to the IgG Fc domain via a linker comprising an amino acid sequence of SEQ ID NO: 12, but having at least one mutation selected from the group consisting of: H216A, K217A, and K219A, relative to SEQ ID NO: 1 in the IL-12p40 portion. In one embodiment, the nucleic acid molecule encodes IL-12p35 of the bivalent homodimeric Fc fused to the IgG Fc domain via a linker comprising an amino acid sequence of SEQ ID NO: 13.

In one embodiment, the nucleic acid molecule encodes one or more IL-12 variant polypeptide comprising bivalent homodimeric Fc comprising (i) the IL-12p40 fused to an IgG Fc domain via a linker and (ii) the IL-12p35 of the bivalent homodimeric Fc. In one embodiment, the nucleic acid molecule encodes one or more IL-12 variant polypeptide comprising bivalent homodimeric Fc comprising (i) the IL-12p35 fused to an IgG Fc domain via a linker and (ii) the IL-12p40 of the bivalent homodimeric Fc.

In one embodiment, the nucleic acid molecule encodes one or more IL-12 variant polypeptide comprising bivalent homodimeric Fc comprising (i) the IL-12p40 fused to an IgG Fc domain via a linker comprising an amino acid sequence of SEQ ID NO: 12 and (ii) the IL-12p35 comprising an amino acid sequence selected from the group consisting of: SEQ ID NO: 2, SEQ ID NO: 30, and SEQ ID NO: 35. In one embodiment, the nucleic acid molecule encodes one or more IL-12 variant polypeptide comprising bivalent homodimeric Fc comprising (i) the IL-12p40 fused to an IgG Fc domain via a linker comprising an amino acid sequence of SEQ ID NO: 12, but having on or more mutations in the IL-12p40 portion and (ii) the IL-12p35 comprising an amino acid sequence selected from the group consisting of: SEQ ID NO: 2, SEQ ID NO: 30, and SEQ ID NO: 35. In one embodiment, the nucleic acid molecule encodes one or more IL-12 variant polypeptide comprising bivalent homodimeric Fc comprising (i) the IL-12p40 fused to an IgG Fc domain via a linker comprising an amino acid sequence of SEQ ID NO: 12, but having at least one mutation selected from the group consisting of: H216X, K217X, and K219X, relative to SEQ ID NO: 1 in the IL-12p40 portion and (ii) the IL-12p35 comprising an amino acid sequence selected from the group consisting of: SEQ ID NO: 2, SEQ ID NO: 30, and SEQ ID NO: 35. In one embodiment, the nucleic acid molecule encodes one or more IL-12 variant polypeptide comprising bivalent homodimeric Fc comprising (i) the IL-12p40 fused to an IgG Fc domain via a linker comprising an amino acid sequence of SEQ ID NO: 12, but having at least one mutation selected from the group consisting of: H216A, K217A, and K219A, relative to SEQ ID NO: 1 in the IL-12p40 portion and (ii) the IL-12p35 comprising an amino acid sequence selected from the group consisting of: SEQ ID NO: 2, SEQ ID NO: 30, and SEQ ID NO: 35.

In one embodiment, the nucleic acid molecule encodes one or more IL-12 variant polypeptide comprising (i) the IL-12p35 fused to an IgG Fc domain via a linker comprising an amino acid sequence of SEQ ID NO: 13 and (ii) the IL-12p40 of the bivalent homodimeric Fc comprising one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the nucleic acid molecule encodes IL-12p40 of the bivalent homodimeric Fc comprising one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

One of skill in the art will recognize that co-expression of either IL-12p40 or IL-12p35 fused to IgG Fc by a linker and the corresponding subunit (IL-12p35 or IL-12p40, respectively) will result in a dimeric IL-12 (i.e. a tetrameric structure comprising a homodimer of heterodimers stabilized by the bivalent IgG Fc domains; see Example 1 and FIG. 5A).

In one embodiment, the nucleic acid molecule encodes an IgG Fc “knob” or an IgG Fc “hole”. In one embodiment, the IgG Fc “knob” and IgG Fc “hole” are variants of IgG Fc. In one embodiment, the nucleic acid molecule encodes IgG Fc, wherein IgG Fc comprises human IgG Fc. In one embodiment, the nucleic acid molecule encodes human IgG Fc, wherein human IgG Fc comprises human IgG1 Fc. In one embodiment, the nucleic acid molecule encodes IgG Fc “knob” comprising the amino acid sequence of SEQ ID NO: 14. In one embodiment, the nucleic acid molecule encodes IgG Fc “hole” comprising the amino acid sequence of SEQ ID NO: 15.

In one embodiment, the nucleic acid molecule encodes IL-12p40 of the bispecific heterodimeric Fc comprising IL-12p40 of WT IL-12. In one embodiment, the nucleic acid molecule encodes IL-12p40 of the bispecific heterodimeric Fc comprising the amino acid sequence of SEQ ID NO: 1 or SEQ ID NO: 34. In one embodiment, the nucleic acid molecule encodes IL-12p40 of the bispecific heterodimeric Fc comprising at least one, at least two, or at least three mutations relative to WT IL-12p40 of SEQ ID NO: 1 or SEQ ID NO: 34. In one embodiment, the nucleic acid molecule encodes IL-12p40 of the bispecific heterodimeric Fc comprising at least one mutation selected from the group consisting of: H216X, K217X, and K219X, relative to SEQ ID NO: 1. In one embodiment, the nucleic acid molecule encodes IL-12p40 of the bispecific heterodimeric Fc comprising at least one mutation selected from the group consisting of: H216A, K217A, and K219A, relative to SEQ ID NO: 1.

In one embodiment, the nucleic acid molecule encodes IL-12p40 of the bispecific heterodimeric Fc comprising one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the nucleic acid molecule encodes IL-12p40 of the bispecific heterodimeric Fc comprising one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In one embodiment, the nucleic acid molecule encodes IL-12p35 of the bispecific heterodimeric Fc comprising IL-12p35 of WT IL-12. In one embodiment, the nucleic acid molecule encodes IL-12p35 of the bispecific heterodimeric Fc comprising a purification tag. In one embodiment, the nucleic acid molecule encodes IL-12p35 of the bispecific heterodimeric Fc comprising the amino acid sequence of SEQ ID NO: 2, SEQ ID NO: 30, or SEQ ID NO: 35.

In one embodiment, the nucleic acid molecule encodes IL-12p40 of the bispecific heterodimeric Fc fused to the IgG Fc “knob” via a linker. In one embodiment, the nucleic acid molecule encodes IL-12p40 of bispecific heterodimeric Fc fused to the IgG Fc “hole” via a linker. In one embodiment, the nucleic acid molecule encodes IL-12p35 of bispecific heterodimeric Fc fused to the IgG Fc “knob” via a linker. In one embodiment, the nucleic acid molecule encodes IL-12p35 of bispecific heterodimeric Fc fused to the IgG Fc “hole” via a linker. In one embodiment, the linker comprises an amino acid sequence of SEQ ID NO: 11.

In one embodiment, the nucleic acid molecule encodes IL-12p40 fused to the IgG Fc “knob” via a linker comprising an amino acid sequence of SEQ ID NO: 16. In one embodiment, the nucleic acid molecule encodes IL-12p40 fused to the IgG Fc “knob” via a linker comprising an amino acid sequence of SEQ ID NO: 16, but having one or more mutations in the IL-12p40 portion. In one embodiment, the nucleic acid molecule encodes IL-12p40 fused to the IgG Fc “knob” via a linker comprising an amino acid sequence of SEQ ID NO: 16, but having at least one mutation selected from the group consisting of: H216X, K217X, and K219X, relative to SEQ ID NO: 1 in the IL-12p40 portion. In one embodiment, the nucleic acid molecule encodes IL-12p40 fused to the IgG Fc “knob” via a linker comprising an amino acid sequence of SEQ ID NO: 16, but having at least one mutation selected from the group consisting of: H216A, K217A, and K219A, relative to SEQ ID NO: 1 in the IL-12p40 portion.

In one embodiment, the nucleic acid molecule encodes IL-12p35 fused to the IgG Fc “hole” via a linker comprising an amino acid sequence of SEQ ID NO: 17.

In one embodiment, the nucleic acid molecule encodes IL-12p40 fused to the IgG Fc “hole” via a linker comprising an amino acid sequence of SEQ ID NO: 18. In one embodiment, the nucleic acid molecule encodes IL-12p40 fused to the IgG Fc “hole” via a linker comprising an amino acid sequence of SEQ ID NO: 18, but having one or more mutations in the IL-12p40 portion. In one embodiment, the nucleic acid molecule encodes IL-12p40 fused to the IgG Fc “hole” via a linker comprising an amino acid sequence of SEQ ID NO: 18, but having at least one mutation selected from the group consisting of: H216X, K217X, and K219X, relative to SEQ ID NO: 1 in the IL-12p40 portion. In one embodiment, the nucleic acid molecule encodes IL-12p40 fused to the IgG Fc “hole” via a linker comprising an amino acid sequence of SEQ ID NO: 18, but having at least one mutation selected from the group consisting of: H216A, K217A, and K219A, relative to SEQ ID NO: 1 in the IL-12p40 portion.

In one embodiment, the nucleic acid molecule encodes IL-12p35 fused to the IgG Fc “knob” via a linker comprising an amino acid sequence of SEQ ID NO: 19.

In one embodiment, the one or more nucleic acid molecules encode a bispecific heterodimeric Fc comprising (i) the IL-12p40 fused to an IgG Fc “knob” via a linker and (ii) the IL-12p35 fused to an IgG Fc “hole” via a linker. In one embodiment, the one or more nucleic acid molecules encode a bispecific heterodimeric Fc comprising (i) the IL-12p35 fused to an IgG Fc “knob” via a linker and (ii) the IL-12p40 fused to an IgG Fc “hole” via a linker.

In one embodiment, the one or more nucleic acid molecules encode a bispecific heterodimeric Fc comprising (i) the IL-12p40 fused to an IgG Fc “knob” via a linker comprising an amino acid sequence of SEQ ID NO: 16 and (ii) the IL-12p35 fused to an IgG Fc “hole” via a linker comprising an amino acid sequence of SEQ ID NO: 17. In one embodiment, the one or more nucleic acid molecules encode a bispecific heterodimeric Fc comprising (i) the IL-12p40 fused to an IgG Fc “knob” via a linker comprising an amino acid sequence of SEQ ID NO: 16, but having one or more mutations in the IL-12p40 portion and (ii) the IL-12p35 fused to an IgG Fc “hole” via a linker comprising an amino acid sequence of SEQ ID NO: 17. In one embodiment, the one or more nucleic acid molecules encode a bispecific heterodimeric Fc comprising (i) the IL-12p40 fused to an IgG Fc “knob” via a linker comprising an amino acid sequence of SEQ ID NO: 16, but having at least one mutation selected from the group consisting of: H216X, K217X, and K219X, relative to SEQ ID NO: 1 in the IL-12p40 portion and (ii) the IL-12p35 fused to an IgG Fc “hole” via a linker comprising an amino acid sequence of SEQ ID NO: 17. In one embodiment, the one or more nucleic acid molecules encode a bispecific heterodimeric Fc comprising (i) the IL-12p40 fused to an IgG Fc “knob” via a linker comprising an amino acid sequence of SEQ ID NO: 16, but having at least one mutation selected from the group consisting of: H216A, K217A, and K219A, relative to SEQ ID NO: 1 in the IL-12p40 portion and (ii) the IL-12p35 fused to an IgG Fc “hole” via a linker comprising an amino acid sequence of SEQ ID NO: 17.

In one embodiment, the one or more nucleic acid molecules encode a bispecific heterodimeric Fc comprising (i) the IL-12p35 fused to an IgG Fc “knob” via a linker comprising an amino acid sequence of SEQ ID NO: 19 and (ii) the IL-12p40 fused to an IgG Fc “hole” via a linker comprising an amino acid sequence of SEQ ID NO: 18. In one embodiment, the one or more nucleic acid molecules encode a bispecific heterodimeric Fc comprising (i) the IL-12p35 fused to an IgG Fc “knob” via a linker comprising an amino acid sequence of SEQ ID NO: 19 and (ii) the IL-12p40 fused to an IgG Fc “hole” via a linker comprising an amino acid sequence of SEQ ID NO: 18, but having one or more mutations in the IL-12p40 portion. In one embodiment, the one or more nucleic acid molecules encode a bispecific heterodimeric Fc comprising (i) the IL-12p35 fused to an IgG Fc “knob” via a linker comprising an amino acid sequence of SEQ ID NO: 19 and (ii) the IL-12p40 fused to an IgG Fc “hole” via a linker comprising an amino acid sequence of SEQ ID NO: 18, but having at least one mutation selected from the group consisting of: H216X, K217X, and K219X. In one embodiment, the one or more nucleic acid molecules encode a bispecific heterodimeric Fc comprising (i) the IL-12p35 fused to an IgG Fc “knob” via a linker comprising an amino acid sequence of SEQ ID NO: 19 and (ii) the IL-12p40 fused to an IgG Fc “hole” via a linker comprising an amino acid sequence of SEQ ID NO: 18, but having at least one mutation selected from the group consisting of: H216A, K217A, and K219A.

One of skill in the art will recognize that co-expression of either IL-12p40 “knob” or IL-12p35 “hole” with the corresponding subunit (IL-12p40 “hole” or IL-12p35 “knob”, respectively) will result in a modified dimeric IL-12, stabilized by the interaction between the “hole” and “knob” IgG Fc domains (see Example 1, FIG. 4A, and FIG. 5B).

In one embodiment, the nucleic acid molecule encodes a single chain bivalent homodimeric Fc. In one embodiment, the nucleic acid molecule encodes a single chain bivalent homodimeric Fc comprising at least two IgG Fc domains. In one embodiment, the IgG is human IgG. In one embodiment, the human IgG is human IgG1. In one embodiment, the nucleic acid molecule encodes human IgG1 Fc domain comprising an amino acid sequence of SEQ ID NO: 10.

In one embodiment, the nucleic acid molecule encodes an IL-12p40 of the single chain bivalent homodimeric Fc comprising IL-12p40 of WT IL-12. In one embodiment, the nucleic acid molecule encodes an IL-12p40 of the single chain bivalent homodimeric Fc comprising the amino acid sequence of SEQ ID NO: 1 or SEQ ID NO: 34. In one embodiment, the nucleic acid molecule encodes an IL-12p40 of the single chain bivalent homodimeric Fc comprising at least one, at least two, or at least three mutations relative to WT IL-12p40 of SEQ ID NO: 1 or SEQ ID NO: 34. In one embodiment, the nucleic acid molecule encodes an IL-12p40 of the single chain bivalent homodimeric Fc comprising at least one mutation selected from the group consisting of: H216X, K217X, and K219X, relative to SEQ ID NO: 1. In one embodiment, the nucleic acid molecule encodes an IL-12p40 of the single chain bivalent homodimeric Fc comprising at least one mutation selected from the group consisting of: H216A, K217A, and K219A, relative to SEQ ID NO: 1.

In one embodiment, the nucleic acid molecule encodes an IL-12p40 of the single chain bivalent homodimeric Fc comprising one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the nucleic acid molecule encodes an IL-12p40 of the single chain bivalent homodimeric Fc comprising one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In one embodiment, the nucleic acid molecule encodes IL-12p35 of the single chain bivalent homodimeric Fc comprising IL-12p35 of WT IL-12. In one embodiment, the nucleic acid molecule encodes IL-12p35 of the single chain bivalent homodimeric Fc comprising a purification tag. In one embodiment, the nucleic acid molecule encodes an IL-12p35 of the single chain bivalent homodimeric Fc comprising the amino acid sequence of SEQ ID NO: 2, SEQ ID NO: 30, or SEQ ID NO: 35.

In one embodiment, the nucleic acid molecule encodes an IL-12p40 of the single chain bivalent homodimeric Fc fused to the IgG Fc domain via a linker. In one embodiment, the nucleic acid molecule encodes IL-12p35 of the single chain bivalent homodimeric Fc fused to the IgG Fc domain via a linker. In one embodiment, the nucleic acid molecule encodes IL-12p40 of the single chain bivalent homodimeric Fc fused to the IL-12p35 of the single chain bivalent homodimeric Fc via a linker.

In one embodiment, the nucleic acid molecule encodes a single chain bivalent homodimeric Fc wherein (i) the IL-12p40 of the single chain bivalent homodimeric Fc is fused to the IL-12p35 of single chain bivalent homodimeric Fc via a linker and (ii) the IL-12p35 of single chain bivalent homodimeric Fc is fused to the IgG Fc domain via a linker. In one embodiment, the nucleic acid molecule encodes a single chain bivalent homodimeric Fc wherein (i) the IL-12p40 of single chain bivalent homodimeric Fc is fused to the IL-12p35 of single chain bivalent homodimeric Fc via a linker and (ii) the IL-12p40 of single chain bivalent homodimeric Fc is fused to the IgG Fc domain via a linker. In one embodiment, the linker comprises one or more amino acid sequence selected from the group consisting of: SEQ ID NO: 20 and SEQ ID NO: 21.

In one embodiment, the nucleic acid molecule encodes one or more IL-12 variant polypeptide comprising a single chain bivalent homodimeric Fc comprising the amino acid sequence of SEQ ID NO: 22. In one embodiment, the nucleic acid molecule encodes one or more IL-12 variant polypeptide comprising a single chain bivalent homodimeric Fc comprises the amino acid sequence of SEQ ID NO: 23. In one embodiment, the nucleic acid molecule encodes one or more IL-12 variant polypeptide comprising a single chain bivalent homodimeric Fc comprising the amino acid sequence of SEQ ID NO: 22, but having one more mutations in the IL-12p40 portion of the single chain bivalent homodimeric Fc. In one embodiment, the nucleic acid molecule encodes one or more IL-12 variant polypeptide comprising a single chain bivalent homodimeric Fc comprising the amino acid sequence of SEQ ID NO: 22, but having at least one mutation selected from the group consisting of: H216X, K217X, and K219X, relative to SEQ ID NO: 1 in the IL-12p40 portion of the single chain bivalent homodimeric Fc. In one embodiment, the nucleic acid molecule encodes one or more IL-12 variant polypeptide comprising a single chain bivalent homodimeric Fc comprising the amino acid sequence of SEQ ID NO: 22, but having at least one mutation selected from the group consisting of: H216A, K217A, and K219A, relative to SEQ ID NO: 1 in the IL-12p40 portion of the single chain bivalent homodimeric Fc. In one embodiment, the nucleic acid molecule encodes one or more IL-12 variant polypeptide comprising a single chain bivalent homodimeric Fc comprising the amino acid sequence of SEQ ID NO: 23, but having one more mutations in the IL-12p40 portion of the single chain bivalent homodimeric Fc. In one embodiment, the nucleic acid molecule encodes one or more IL-12 variant polypeptide comprising a single chain bivalent homodimeric Fc comprising the amino acid sequence of SEQ ID NO: 23, but having at least one mutation selected from the group consisting of: H216X, K217X, and K219X, relative to SEQ ID NO: 1 in the IL-12p40 portion of the single chain bivalent homodimeric Fc. In one embodiment, the nucleic acid molecule encodes one or more IL-12 variant polypeptide comprising a single chain bivalent homodimeric Fc comprising the amino acid sequence of SEQ ID NO: 23, but having at least one mutation selected from the group consisting of: H216A, K217A, and K219A, relative to SEQ ID NO: 1 in the IL-12p40 portion of the single chain bivalent homodimeric Fc.

One of skill in the art will recognize that expression of either configuration of single chain bivalent homodimeric Fc will result in a dimeric IL-12 (i.e. a dimer of fused dimers stabilized by the bivalent IgG Fc domains; see Example 1 and FIG. 5B).

In one embodiment, the present disclosure provides one or more nucleic acid molecules encoding one or more IL-12 variant polypeptide comprising a single chain monomeric IL-12 and a bispecific heterodimeric Fc.

In one embodiment, the bispecific heterodimeric Fc comprises an IgG Fc “knob” and an IgG Fc “hole”. In one embodiment, the IgG Fc “knob” and IgG Fc “hole” are variants of IgG Fc. In one embodiment, the IgG Fc comprises human IgG Fc. In one embodiment, the human IgG Fc comprises human IgG1 Fc. In one embodiment, the nucleic acid molecule encodes IgG Fc “hole” fused to signal peptide via a linker. In one embodiment, the nucleic acid molecule encodes IgG Fc “knob” fused to a signal peptide via a linker. In one embodiment, the linker comprises an amino acid sequence of SEQ ID NO: 11.

In one embodiment, the nucleic acid molecule encodes IL-12p40 of the single chain monomeric IL-12 comprising IL-12p40 of WT IL-12. In one embodiment, the nucleic acid molecule encodes IL-12p40 of the single chain monomeric IL-12 comprising the amino acid sequence of SEQ ID NO: 1 or SEQ ID NO: 34. In one embodiment, the nucleic acid molecule encodes IL-12p40 of the single chain monomeric IL-12 comprising at least one, at least two, or at least three mutations relative to WT IL-12p40 of SEQ ID NO: 1 or SEQ ID NO: 34. In one embodiment, the nucleic acid molecule encodes IL-12p40 of the single chain monomeric IL-12 comprising at least one mutation selected from the group consisting of: H216X, K217X, and K219X, relative to SEQ ID NO: 1. In one embodiment, the nucleic acid molecule encodes IL-12p40 of the single chain monomeric IL-12 comprising at least one mutation selected from the group consisting of: H216A, K217A, and K219A, relative to SEQ ID NO: 1.

In one embodiment, the nucleic acid molecule encodes IL-12p40 of the single chain monomeric IL-12 comprising one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the nucleic acid molecule encodes IL-12p40 of the single chain monomeric IL-12 comprising one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In one embodiment, the nucleic acid molecule encodes IL-12p35 of the single chain monomeric IL-12 comprising IL-12p35 of WT IL-12. In one embodiment, the nucleic acid molecule encodes IL-12p35 of the single chain monomeric IL-12 comprising a purification tag. In one embodiment, the nucleic acid molecule encodes IL-12p35 of the single chain monomeric IL-12 comprising the amino acid sequence of SEQ ID NO: 2, SEQ ID NO: 30, or SEQ ID NO: 35.

In one embodiment, the nucleic acid molecule encodes a single chain monomeric IL-12 comprising the IL-12p40 fused via a linker to the IL-12p35. In one embodiment, the nucleic acid molecule encodes a single chain monomeric IL-12 comprising (i) the IL-12p40 fused via a linker to the IL-12p35 and (ii) the IL-12p35 fused via a linker to IgG Fc “knob”. In one embodiment, the nucleic acid molecule encodes a single chain monomeric IL-12 comprising (i) the IL-12p35 fused via a linker to the IL-12p40 and (ii) the IL-12p40 fused via a linker to IgG Fc “knob”. In one embodiment, the nucleic acid molecule encodes a single chain monomeric IL-12 comprising (i) the IL-12p40 fused via a linker to the IL-12p35 and (ii) the IL-12p35 fused via a linker to IgG Fc “hole”. In one embodiment, the nucleic acid molecule encodes a single chain monomeric IL-12 comprising (i) the IL-12p35 fused via a linker to the IL-12p40 and (ii) the IL-12p40 fused via a linker to IgG Fc “hole”. In one embodiment, the linker comprises one or more amino acid sequence selected from the group consisting of: SEQ ID NO: 20 and SEQ ID NO: 21.

In one embodiment, the nucleic acid molecule encodes a single chain monomeric IL-12 comprising an amino acid sequence of one or more selected from the group consisting of: SEQ ID NO: 26, SEQ ID NO: 27, SEQ ID NO: 28 and SEQ ID NO: 29. In one embodiment, the nucleic acid molecule encodes one or more single chain monomeric IL-12 comprising an amino acid sequence of one or more selected from the group consisting of: SEQ ID NO: 26, SEQ ID NO: 27, SEQ ID NO: 28 and SEQ ID NO: 29, but having one more mutations in the IL-12p40 portion of the single chain monomeric IL-12. In one embodiment, the nucleic acid molecule encodes one or more single chain monomeric IL-12 comprising an amino acid sequence of one or more selected from the group consisting of: SEQ ID NO: 26, SEQ ID NO: 27, SEQ ID NO: 28 and SEQ ID NO: 29, but having at least one mutation selected from the group consisting of: H216X, K217X, and K219X, relative to SEQ ID NO: 1 in the IL-12p40 portion of the single chain monomeric IL-12. In one embodiment, the nucleic acid molecule encodes one or more single chain monomeric IL-12 comprising an amino acid sequence of one or more selected from the group consisting of: SEQ ID NO: 26, SEQ ID NO: 27, SEQ ID NO: 28 and SEQ ID NO: 29, but having at least one mutation selected from the group consisting of: H216A, K217A, and K219A, relative to SEQ ID NO: 1 in the IL-12p40 portion of the single chain monomeric IL-12.

In one embodiment, the nucleic acid molecule encodes a single chain monomeric IL-12 comprising an amino acid sequence having 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more sequence identity to at least one selected from the group consisting of: of: SEQ ID NO: 26, SEQ ID NO: 27, SEQ ID NO: 28 and SEQ ID NO: 29.

In one embodiment, the one or more nucleic acid molecules encode (i) the single chain monomeric IL-12 comprising an amino acid sequence of SEQ ID NO: 26 and (ii) the IgG Fc “hole” comprising an amino acid sequence of SEQ ID NO: 25.

In one embodiment, the one or more nucleic acid molecules encode (i) the single chain monomeric IL-12 comprising an amino acid sequence of SEQ ID NO: 27 and (ii) the IgG Fc “hole” comprising an amino acid sequence of SEQ ID NO: 25.

In one embodiment, the one or more nucleic acid molecules encode (i) the single chain monomeric IL-12 comprising an amino acid sequence of SEQ ID NO: 28 and (ii) the IgG Fc “knob” comprising an amino acid sequence of SEQ ID NO: 24.

In one embodiment, the one or more nucleic acid molecules encode (i) the single chain monomeric IL-12 comprising an amino acid sequence of SEQ ID NO: 27 and (ii) the IgG Fc “knob” comprising an amino acid sequence of SEQ ID NO: 24.

One of skill in the art will recognize the co-expression of an IgG Fc “knob” or “hole” with a single chain IL-12 fused to the corresponding IgG Fc “hole” or “knob” respectively, will result in a monomeric IL-12 stabilized by a heterodimeric Fc (see Example 1 and FIG. 5C).

In one embodiment, the one or more nucleic acid molecules encode a dimeric IL-12 and a bispecific heterodimeric Fc.

In one embodiment, the bispecific heterodimeric Fc comprises an IgG Fc “knob” and an IgG Fc “hole”. In one embodiment, the IgG Fc “knob” and IgG Fc “hole” are variants of IgG Fc. In one embodiment, the IgG Fc comprises human IgG Fc. In one embodiment, the human IgG Fc comprises human IgG1 Fc. In one embodiment, the IgG Fc “hole” is fused to signal peptide via a linker. In one embodiment, the IgG Fc “knob” is fused to a signal peptide via a linker. In one embodiment, the linker comprises an amino acid sequence of SEQ ID NO: 11.

In one embodiment, the nucleic acid molecule encodes an IL-12p40 of the dimeric IL-12 comprising IL-12p40 of WT IL-12. In one embodiment, the nucleic acid molecule encodes anIL-12p40 of the dimeric IL-12 comprising the amino acid sequence of SEQ ID NO: 1 or SEQ ID NO: 34. In one embodiment, the nucleic acid molecule encodes an IL-12p40 of the dimeric IL-12 comprising at least one, at least two, or at least three mutations relative to WT IL-12p40 of SEQ ID NO: 1 or SEQ ID NO: 34. In one embodiment, the nucleic acid molecule encodes an IL-12p40 of the dimeric IL-12 comprising at least one mutation selected from the group consisting of: H216X, K217X, and K219X, relative to SEQ ID NO: 1. In one embodiment, the nucleic acid molecule encodes an IL-12p40 of the dimeric IL-12 comprising at least one mutation selected from the group consisting of: H216A, K217A, and K219A, relative to SEQ ID NO: 1.

In one embodiment, the nucleic acid molecule encodes an IL-12p40 of the dimeric IL-12 comprising one or more amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

In one embodiment, the nucleic acid molecule encodes an IL-12p40 of the dimeric IL-12 comprising one or more amino acid sequence selected from the group consisting of: SEQ ID NO: 3, SEQ ID NO: 4, SEQ ID NO: 5, SEQ ID NO: 6, SEQ ID NO: 7, SEQ ID NO: 8, SEQ ID NO: 9, wherein amino acid residues 1-22 comprising the sequence MCHQQLVISWFSLVFLASPLVA (SEQ ID NO: 31) are replaced with one or more different signal peptide. In one embodiment, the one or more different signal peptide is selected from the group consisting of: SEQ ID NO: 32 and SEQ ID NO: 33.

In one embodiment, the nucleic acid molecule encodes an IL-12p35 of the dimeric IL-12 comprising IL-12p35 of WT IL-12. In one embodiment, the nucleic acid molecule encodes IL-12p35 of the dimeric IL-12 comprising a purification tag. In one embodiment, the nucleic acid molecule encodes an IL-12p35 of the dimeric IL-12 comprising the amino acid sequence of SEQ ID NO: 2, SEQ ID NO: 30, or SEQ ID NO: 35.

In one embodiment, the nucleic acid molecule encodes a dimeric IL-12 comprising (i) the IL-12p40 of the dimeric IL-12 and (ii) the IL-12p35 fused via a linker to IgG Fc “knob”. In one embodiment, the nucleic acid molecule encodes a dimeric IL-12 comprising (i) the IL-12p40 of the dimeric IL-12 and (ii) the IL-12p35 fused via a linker to IgG Fc “hole”. In one embodiment, the nucleic acid molecule encodes a dimeric IL-12 comprising (i) the IL-12p35 of the dimeric IL-12 and (ii) the IL-12p40 fused via a linker to IgG Fc “knob”. In one embodiment, the nucleic acid molecule encodes a dimeric IL-12 comprising (i) the IL-12p35 of the dimeric IL-12 and (ii) the IL-12p40 fused via a linker to IgG Fc “hole”.

In one embodiment, the one or more nucleic acid molecules encode (i) the IL-12p40 of the dimeric IL-12, (ii) the IL-12p35 fused via a linker to IgG Fc “knob”, and (iii) the IgG Fc “hole”. In one embodiment, the one or more nucleic acid molecules encode (i) the IL-12p35 of the dimeric IL-12, (ii) the IL-12p40 fused via a linker to IgG Fc “knob”, and (iii) the IgG Fc “hole”. In one embodiment, the one or more nucleic acid molecules encode (i) the IL-12p40 of the dimeric IL-12, (ii) the IL-12p35 fused via a linker to IgG Fc “hole”, and (iii) the IgG Fc “knob”. In one embodiment the one or more nucleic acid molecules encode (i) the IL-12p35 of the dimeric IL-12, (ii) the IL-12p40 fused via a linker to IgG Fc “hole”, and (iii) the IgG Fc “knob”.

In one embodiment, the one or more nucleic acid molecules encode (i) the IL-12p40 comprising an amino acid sequence of one or more selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34, (ii) the IL-12p35 fused via a linker to IgG Fc “knob” comprising an amino acid sequence of SEQ ID NO: 19, and (iii) the IgG Fc “hole” comprising an amino acid sequence of SEQ ID NO: 25.

In one embodiment, the one or more nucleic acid molecules encode (i) the IL-12p40 comprising an amino acid sequence of one or more selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide selected from the group consisting of: SEQ ID NO: 32 and SEQ ID NO: 33, (ii) the IL-12p35 fused via a linker to IgG Fc “knob” comprising an amino acid sequence of SEQ ID NO: 19, and (iii) the IgG Fc “hole” comprising an amino acid sequence of SEQ ID NO: 25.

In one embodiment, the one or more nucleic acid molecules encode (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “knob” comprising an amino acid sequence of SEQ ID NO: 16, and (iii) the IgG Fc “hole” comprising an amino acid sequence of SEQ ID NO: 25. In one embodiment, the one or more nucleic acid molecules encode (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “knob” comprising an amino acid sequence of SEQ ID NO: 16, but having one or more mutations in the IL-12p40 portion, and (iii) the IgG Fc “hole” comprising an amino acid sequence of SEQ ID NO: 25. In one embodiment, the one or more nucleic acid molecules encode (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “knob” comprising an amino acid sequence of SEQ ID NO: 16, but having at least one mutation selected from the group consisting of: H216X, K217X, and K219X, relative to SEQ ID NO: 1 in the IL-12p40 portion and (iii) the IgG Fc “hole” comprising an amino acid sequence of SEQ ID NO: 25. In one embodiment, the one or more nucleic acid molecules encode (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “knob” comprising an amino acid sequence of SEQ ID NO: 16, but having at least one mutation selected from the group consisting of: H216A, K217A, and K219A, relative to SEQ ID NO: 1 in the IL-12p40 portion and (iii) the IgG Fc “hole” comprising an amino acid sequence of SEQ ID NO: 25.

In one embodiment, the one or more nucleic acid molecules encode (i) the IL-12p40 comprising an amino acid sequence of one or more selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34, (ii) the IL-12p35 fused via a linker to IgG Fc “hole” comprising an amino acid sequence of SEQ ID NO: 17, and (iii) the IgG Fc “knob” comprising an amino acid sequence of SEQ ID NO: 24.

In one embodiment, the one or more nucleic acid molecules encode (i) the IL-12p40 comprising an amino acid sequence of one or more selected from the group consisting of: SEQ ID NO: 3, SEQ ID NO: 4, SEQ ID NO: 5, SEQ ID NO: 6, SEQ ID NO: 7, SEQ ID NO: 8, and SEQ ID NO: 9, wherein amino acid residues 1-22 comprising the sequence MCHQQLVISWFSLVFLASPLVA (SEQ ID NO: 31) are replaced with one or more different signal peptide selected from the group consisting of: SEQ ID NO: 32 and SEQ ID NO: 33, (ii) the IL-12p35 fused via a linker to IgG Fc “hole” comprising an amino acid sequence of SEQ ID NO: 17, and (iii) the IgG Fc “knob” comprising an amino acid sequence of SEQ ID NO: 24.

In one embodiment, the one or more nucleic acid molecules encode (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “hole” comprising an amino acid sequence of SEQ ID NO: 18, and (iii) the IgG Fc “knob” comprising an amino acid sequence of SEQ ID NO: 24. In one embodiment, the one or more nucleic acid molecules encode (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “hole” comprising an amino acid sequence of SEQ ID NO: 18, but having at least one mutation in the IL-12p40 portion, and (iii) the IgG Fc “knob” comprising an amino acid sequence of SEQ ID NO: 24. In one embodiment, the one or more nucleic acid molecules encode (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “hole” comprising an amino acid sequence of SEQ ID NO: 18, but having at least one mutation selected from the group consisting of: H216X, K217X, and K219X, relative to SEQ ID NO: 1 in the IL-12p40 portion, and (iii) the IgG Fc “knob” comprising an amino acid sequence of SEQ ID NO: 24. In one embodiment, the one or more nucleic acid molecules encode (i) the IL-12p35 comprising an amino acid sequence selected from the group consisting of: SEQ ID NO: 2, SEQ ID NO: 30, and SEQ ID NO: 35, (ii) the IL-12p40 fused via a linker to IgG Fc “hole” comprising an amino acid sequence of SEQ ID NO: 18, but having at least one mutation selected from the group consisting of: H216A, K217A, and K219A, relative to SEQ ID NO: 1 in the IL-12p40 portion, and (iii) the IgG Fc “knob” comprising an amino acid sequence of SEQ ID NO: 24.

One of skill in the art will recognize that co-expression of IL-12p40 or IL-12p35 with (i) the corresponding subunit fused to IgG Fc “hole” or “knob” and (ii) the corresponding IgG Fc “knob” or “hole” will result in a dimeric IL-12 stabilized by heterodimeric IgG Fc (see Example 1 and FIG. 5D).

The nucleic acid molecule(s) encoding the polypeptide(s) of the present disclosure can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The nucleic acid molecule(s) may comprise any type of nucleic acid, including, but not limited to DNA and RNA. For example, in one embodiment, the composition comprises an isolated DNA molecule, including for example, an isolated cDNA molecule, encoding the polypeptide(s) of the disclosure. In one embodiment, the composition comprises an isolated RNA molecule encoding the polypeptide(s) of the disclosure, or a functional fragment thereof.

The nucleic acid molecule(s) of the present disclosure can be modified to improve stability in serum or in growth medium for cell cultures. Modifications can be added to enhance stability, functionality, and/or specificity and to minimize immunostimulatory properties of the nucleic acid molecule of the disclosure. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present disclosure the nucleic acid molecule(s) may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH2, NHR, NR2 or ON, wherein R is C1-C6 alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the disclosure can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

In certain embodiments, the nucleic acid molecule(s) of the disclosure has one or more of the following properties:

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, or as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, or different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the disclosure may be present at one or more of, a phosphate group, a sugar group, backbone, N-terminus, C-terminus, or nucleobase.

The present disclosure also includes a vector in which the nucleic acid molecule(s) of the present disclosure is/are inserted. The art is replete with suitable vectors that are useful in the present disclosure.

In brief summary, the expression of natural or synthetic nucleic acids encoding a fusion protein of the disclosure is typically achieved by operably linking a nucleic acid encoding the fusion protein of the disclosure or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the disclosure provides a gene therapy vector.

The isolated nucleic acid of the disclosure can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses (AAV), herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

### Methods

In various embodiments, the present disclosure relates to methods of administering to a subject one or more IL-12 variant polypeptide of the present disclosure or one or more nucleic acid molecule encoding one or more IL-12 variant polypeptide of the present disclosure, as described above. In some embodiments, the present disclosure relates to methods of treating or preventing one or more disease or disorder in a subject, comprising administering to the subject one or more IL-12 variant polypeptide of the present disclosure or one or more nucleic acid molecule encoding one or more IL-12 variant polypeptide of the present disclosure, as described above.

### Methods of Administration

In one embodiment, the present disclosure comprises a method of administering to a subject one or more composition of the present disclosure, as described above. In one embodiment, the composition comprises one or more IL-12 variant polypeptide, wherein the one or more IL-12 variant polypeptide specifically binds to IL-12 receptor β2 (IL-12Rβ2) and wherein the one or more IL-12 variant polypeptide exhibits substantially reduced binding to IL-12 receptor β1 (IL-12Rβ1). In one embodiment, the composition comprises one or more nucleic acid molecule encoding one or more IL-12 variant polypeptide, wherein the one or more IL-12 variant polypeptide specifically binds to IL-12 receptor β2 (IL-12Rβ2) and wherein the one or more IL-12 variant polypeptide exhibits substantially reduced binding to IL-12 receptor β1 (IL-12Rβ1).

In some embodiments, the subject has a disease or disorder that can be treated by reducing the maximal level of agonism through the IL-12 receptors. In some embodiments, the subject is at risk of developing a disease or disorder that can be prevented by reducing the maximal level of agonism through IL-12 receptors. In some embodiments, the disease or disorder is cancer. Non-limiting examples of types of cancers that can benefit from administration of one or more composition of the present disclosure are disclosed elsewhere herein.

The present disclosure encompasses the preparation and use of pharmaceutical compositions for administration comprising a composition of the present disclosure, disclosed herein, as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art. In various embodiments, the active ingredient is one or more nucleic acid molecule, one or more polypeptide, or a combination thereof, as elsewhere described herein. The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In some embodiments, pharmaceutical compositions can include large, slowly metabolized macromolecules such as proteins, polysaccharides such as chitosan, polylactic acids, polyglycolic acids and copolymers (such as latex functionalized Sepharose™ agarose, cellulose, and the like), polymeric amino acids, amino acid copolymers, and lipid aggregates (such as oil droplets or liposomes).

The pharmaceutical compositions useful for practicing the disclosure may be administered to deliver a dose of between about 0.1 ng/kg/day and 100 mg/kg/day, or more.

In various embodiments, the pharmaceutical compositions useful in the methods of the disclosure may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, other preparations containing the active ingredient, and immunologically based systems may also be used to administer an appropriate modulator thereof, according to the methods of the disclosure.

A carrier may bear a subject agent (e.g., one or more IL-12 variant polypeptide) in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear one or more IL-12 variant polypeptide by non-covalent associations, such as non-covalent bonding or by encapsulation. The nature of the carrier can be either soluble or insoluble for purposes of the disclosure.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S.

### Food and Drug Administration.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the disclosure may be prepared, packaged, or sold in formulations suitable for oral, rectal, vaginal, parenteral, topical, pulmonary, intranasal, buccal, intravenous, transdermal, intralesional, subcutaneous, intramuscular, ophthalmic, intrathecal and other known routes of administration. Other contemplated formulations include projected nanoparticles, liposomal preparations, other preparations containing the active ingredient, and immunologically-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

A formulation of a pharmaceutical composition of the disclosure suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

Pharmaceutically acceptable excipients used in the manufacture of pharmaceutical compositions include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Liquid formulations of a pharmaceutical composition of the disclosure may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, or in certain embodiments from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In certain embodiments, such powders comprise particles wherein at least 98% of the particles by weight have a diameter greater than 0.5 nanometers and at least 95% of the particles by number have a diameter less than 7 nanometers. In certain embodiments, at least 95% of the particles by weight have a diameter greater than 1 nanometer and at least 90% of the particles by number have a diameter less than 6 nanometers. In certain embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some instances having a particle size of the same order as particles comprising the active ingredient).

Pharmaceutical compositions of the disclosure formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. In certain embodiments, the droplets provided by this route of administration have an average diameter in the range from about 0.1 to about 200 nanometers. The formulations described herein as being useful for pulmonary delivery are also useful for intranasal delivery of a pharmaceutical composition of the disclosure. Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the disclosure may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. In certain embodiments, such powdered, aerosolized, or aerosolized formulations, when dispersed, have an average particle or droplet size in the range from about 0.1 to about 200 nanometers, and may further comprise one or more of the additional ingredients described herein.

A pharmaceutical composition of the disclosure may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Genaro, ed., 1985, Remington's Pharmaceutical Sciences, Mack Publishing Co., Easton, Pa., which is incorporated herein by reference.

Typical dosages of the compound of the disclosure which may be administered to an animal (e.g. a human) range in amount from about 0.001 mg to about 1000 mg per kilogram of body weight of the animal. The precise dosage administered will vary depending upon any number of factors, including, but not limited to, the type of animal and type of disease or disorder being treated, the age of the animal and the route of administration. In some embodiments, the dosage of the compound will vary from about 0.1 mg to about 10 mg per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease or disorder being treated, the type and age of the animal, etc.

### Methods of Treatment/Prevention

In one embodiment, the present disclosure comprises a method of treating or preventing one or more disease or disorder in a subject in need thereof, comprising administering one or more composition of the present disclosure, as described above. In one embodiment, the method comprises administering to the subject a composition comprising one or more IL-12 variant polypeptide, wherein the one or more IL-12 variant polypeptide specifically binds to IL-12 receptor β2 (IL-12Rβ2) and wherein the one or more IL-12 variant polypeptide exhibits substantially reduced binding to IL-12 receptor 31 (IL-12Rβ1), as described above. In one embodiment, the method comprises administering to the subject a composition comprising one or more nucleic acid molecule encoding one or more IL-12 variant polypeptide, wherein the one or more IL-12 variant polypeptide specifically binds to IL-12 receptor β2 (IL-12Rβ2) and wherein the one or more IL-12 variant polypeptide exhibits substantially reduced binding to IL-12 receptor 31 (IL-12Rβ1), as described above.

In some embodiments, a composition of the disclosure, as described above, is administered to a cell, tissue, organ, system, or subject to treat or prevent a disease or disorder. One skilled in the art, based upon the disclosure provided herein, would understand that the disclosure is useful in subjects who, in whole (e.g., systemically) or in part (e.g., locally, cell, tissue, organ), are being or will be, treated for a disease or disorder where a reduction in maximal IL-12 signaling activity would be beneficial. The skilled artisan will appreciate, based upon the teachings provided herein, that the diseases and disorders treatable by the compositions and methods described herein encompass any disease or disorder wherein a reduction in maximal IL-12 signaling activity will promote a positive biologic, physiologic, clinical or therapeutic outcome. One of skill in the art will also appreciate administration can be acute (e.g., over a short period of time, such as a day, a week or a month) or chronic (e.g., over a long period of time, such as several months or a year or more).

It will be appreciated by one of skill in the art, when armed with the present disclosure including the methods detailed herein, that the disclosure is not limited to treatment of a disease or disorder once it is established. Particularly, the symptoms of the disease or disorder need not have manifested to the point of detriment to the subject; indeed, the disease or disorder need not be detected in a subject before treatment is administered. That is, significant pathology from disease or disorder does not have to occur before the present disclosure may provide benefit. Therefore, the present disclosure, as described more fully herein, includes a method for preventing diseases and disorders in a subject, in that one or more IL-12 variant polypeptide, wherein the one or more IL-12 variant polypeptide specifically binds to IL-12Rβ2 but exhibits substantially reduced binding to IL-12Rβ1, as discussed elsewhere herein, can be administered to a subject prior to the onset of the disease or disorder, thereby preventing the disease or disorder from developing.

In one embodiment, the one or more disease or disorder comprises cancer. One of skill in the art will recognize that the compositions of the present disclosure can be administered to a subject having cancer to treat the cancer or a subject at risk of developing cancer to prevent the cancer. Non-limiting examples of types of cancers that can be treated or prevented by the methods and compositions of the disclosure include solid tumor cancers, liquid cancers, blood cancers, teratomas, sarcomas, and carcinomas. The following are non-limiting examples of cancers that can be treated or prevented by the methods and compositions of the disclosure: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and Wilms Tumor.

In some embodiments, the methods of the present disclosure is useful for treating or preventing a tumor or cancer that is resistant to immune checkpoint inhibitors (ICIs). Exemplary immune checkpoint inhibitors include, but is not limited to, anti-PD1 (e.g., nivolumab), anti-CTLA4 (e.g., ipilimumab), anti-TIM3, anti-TIGIT, anti-LAG3, anti-B7H3, anti-B7H4, anti-VISTA, anti-ICOS, anti-GITR, anti-41BB, anti-OX40, and anti-CD40. Examples of targets of immune checkpoint inhibitors include but are not limited to: PD-L1, PD1, CTLA4, TIM3, TIGIT, LAG3, B7H3, B7H4, VISTA, ICOS, GITR, 41BB, OX40, and CD40. Thus, examples of immune checkpoint inhibitors include agents that inhibit proteins such as: PD-L1, PD1, CTLA4, TIM3, TIGIT, LAG3, B7H3, B7H4, VISTA, ICOS, GITR, 41BB, OX40, or CD40. In some cases, one or more IL-12 variant polypeptide is co-administered with an immune checkpoint inhibitor (e.g., an agent that inhibits PD-L1, PD1, CTLA4, TIM3, TIGIT, LAG3, B7H3, B7H4, VISTA, ICOS, GITR, 41BB, OX40, or CD40, or any combination thereof).

In some embodiments, the method comprises co-administering one or more composition of the present disclosure with one or more additional agent. In some embodiments, the agents are present in the cell or in the subject's body at the same time or exert their biological or therapeutic effect at the same time. In some embodiments, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

In some embodiments, the one or more additional agent comprises one or more selected from the group consisting of: a chemical compound, a polypeptide, a peptide, a peptidomimetic, an antibody, a cytokine, a nucleic acid molecule, a ribozyme, a small molecule chemical compound, and an antisense nucleic acid molecule. In some embodiments, the one or more additional agent comprises one or more cancer therapeutic agent (e.g. a chemotherapeutic), or cancer immunotherapeutic agent (e.g. a cancer-directed antibody). Such administration may involve concurrent (i.e. at the same time), prior, or subsequent administration of the drug/antibody with respect to the administration of an agent or agents of the disclosure. A person of ordinary skill in the art would have no difficulty determining the appropriate timing, sequence and dosages of administration for particular drugs and compositions of the present disclosure.

In some embodiments, the cancer therapeutic agent comprises a chemotherapeutic agent. Exemplary chemotherapeutic agents that can be administered in conjunction with the compositions of the present disclosure to treat or prevent cancer include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine and vinorelbine tartrate.

In some embodiments, the cancer immunotherapeutic agent comprises an agent that opsonizes a target cell. An agent that opsonizes a target cell (an “opsonizing agent”) is any agent that can bind to a target cell (e.g., a cancer cell) and opsonize the target cell (e.g., mark the target cell for phagocytosis and/or for antibody-dependent cell mediated cytotoxicity (ADCC)). For example, any antibody that can bind to a target cell (e.g., a cancer cell such as a tumor cell), where the antibody has an FC region, is considered to be an agent that opsonizes a target cell. In some cases, the agent that opsonizes a target cell is an antibody that binds to a target cell (e.g., an anti-tumor antibody, an anti-cancer antibody, and the like). In one embodiment, that agent that opsonizes the target cell is Rituximab. Rituximab is a chimeric unconjugated monoclonal antibody directed at the CD20 antigen. CD20 has an important functional role in B cell activation, proliferation, and differentiation. In one embodiment, that agent that opsonizes the target cell is Cetuximab. Cetuximab binds to the EGF receptor (EGFR), and has been used in the treatment of solid tumors including colon cancer and squamous cell carcinoma of the head and neck (SCCHN).

In some embodiments, the cancer immunotherapeutic agent comprises a specific antibody. Exemplary antibodies selective for tumor cell markers, radiation, surgery, and/or hormone deprivation, see Kwon et al., Proc. Natl. Acad. Sci U.S.A., 96: 15074-9, 1999. Angiogenesis inhibitors can also be combined with the methods of the disclosure. A number of antibodies are currently in clinical use for the treatment of cancer, and others are in varying stages of clinical development. For example, there are a number of antigens and corresponding monoclonal antibodies for the treatment of B cell malignancies. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Two new monoclonal antibodies targeting CD20, tositumomab and ibritumomab, have been submitted to the Food and Drug Administration (FDA). These antibodies are conjugated with radioisotopes. Alemtuzumab (Campath) is used in the treatment of chronic lymphocytic leukemia; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer.

Monoclonal antibodies useful in the methods of the disclosure that have been used in solid tumors include, without limitation, edrecolomab and trastuzumab (herceptin). Edrecolomab targets the 17-1A antigen seen in colon and rectal cancer, and has been approved for use in Europe for these indications. Trastuzumab targets the HER-2/neu antigen.

In one embodiment, the cancer immunotherapeutic agent is one or more selected from the group consisting of: cetuximab (binds EGFR), panitumumab (binds EGFR), rituximab (binds CD20), trastuzumab (binds HER2), pertuzumab (binds HER2), alemtuzumab (binds CD52), brentuximab (binds CD30), tositumomab, ibritumomab, gemtuzumab, ibritumomab, and edrecolomab (binds 17-1A), and a combination thereof.

In one embodiment, the cancer immunotherapeutic agent comprises an antigen binding region that targets one or more selected from the group consisting of: CD19, CD20, CD22, CD24, CD25, CD30, CD33, CD37, CD38, CD44, CD45, CD47, CD51, CD52, CD56, CD62L, CD70, CD74, CD79, CD80, CD96, CD97, CD99, CD123, CD134, CD138, CD152 (CTLA-4), CD200, CD213A2, CD221, CD248, CD276 (B7-H3), B7-H4, CD279 (PD-1), CD274 (PD-L1), CD319, EGFR, EPCAM, 17-1A, HER1, HER2, HER3, CD117, C-Met, HGFR, PDGFRA, AXL, TWEAKR, PTHR2, HAVCR2 (TIM3), GD2 ganglioside, MUC1, mucin CanAg, mesothelin, endoglin, Lewis-Y antigen, CEA, CEACAM1, CEACAM5, CA-125, PSMA, BAFF, FGFR2, TAG-72, gelatinase B, glypican 3, nectin-4, BCMA, CSF1R, SLAMF7, integrin αᵥβ₃, TYRP1, GPNMB, CLDN18.2, FOLR1, CCR4, CXCR4, MICA, C242 antigen, DLL3, DLL4, EGFL7, vimentin, fibronectin extra domain-B, TROP-2, LRRC15, FAP, SLITRK6, NOTCH2, NOTCH3, Tenascin-3, STEAP1, and NRP1.

In one embodiment, the cancer immunotherapeutic agent comprises an antigen binding region that targets one or more selected from the group consisting of: CD19, CD20, CD22, CD24, CD25, CD30, CD33, CD38, CD44, CD47, SIRPA, CD52, CD56, CD70, CD96, CD97, CD99, CD123, CD279 (PD-1), CD274 (PD-L1), EGFR, 17-1A, HER2, CD117, C-Met, PTHR2, and HAVCR2 (TIM3).

In some embodiments, the cancer immunotherapeutic agent comprises an immunomodulatory agent. In some embodiments, the immunomodulatory agent includes, but not limited to, an anti-CTLA4 antibody, an anti-PD-1 antibody, an anti-PD-L1 antibody, a TIGIT antibody, a TIM3 antibody, a LAG3 antibody, a VISTA antibody, a B7H3 antibody, a B7H4 antibody, a CD40 agonist, a 4-1BB modulator (e.g., a 41BB-agonist), an OX-40 modulator (e.g., an OX-40 agonist), a GITR modulator (e.g., a GITR agonist), a CD47 binding agent such as an anti-CD47 antibody or a high affinity CD47 binding agent, a SIRPA binding agent such as an anti-SIRPA antibody or high affinity SIRPA binding agent, and the like), a TGFbeta antagonist such as an anti-TGFbeta antibody, a cytokine or a cytokine variant including IL-1, IL-2, IL-10, IL-15, IL-18, IL-21, IL-33, Interferon alpha, Interferon beta, Interferon gamma, TNF, TRAIL, lymphotoxin, LIGHT/TNSF14, or an agonist of a Toll Like Receptor including TLR2, TLR4, TLR5, TLR7, TLR9, an agonist of an inflammasome, an agonist of the STING/cGAS pathway, or an agonist of the RIG-I pathway, an antagonist of the adenosine receptors A2aR/A2bR, an antagonist of the Aryl hydrocarbon receptor, an antagonist of IDO and/or TDO, or an oncolytic virus.

The IL-12 variant polypeptides can be administered in combination with, or as a fusion with, immune checkpoint inhibitors and/or tumor opsonizing antibodies. The subject polypeptides can be administered in combination with cell therapies such as chimeric antigen receptor T cell (CAR-T cell), TCR-T cell, chimeric antigen receptor NK cell (CAR-NK cell), and Tumor-infiltrating Lymphocyte (TIL) therapies. The subject polypeptides can also be administered as part of a cell therapy, e.g., can be a protein secreted by a cell, e.g., TRUCK cells (“T cells redirected for antigen-unrestricted cytokine-initiated killing”), which are a version of CAR-T cells, CAR-NK cells, TIL cells, or T or NK cell transduced with an engineered T cell receptor, and the like. In other embodiments, the one or more IL-12 variant polypeptide is co-administered with an oncolytic virus.

In some embodiments, one or more nucleic acid encoding one or more IL-12 variant polypeptide is included within an engineered (“altered”) immune cell such as a CAR-T or CAR-NK cell or T or NK cell transduced with an engineered T cell receptor. In this instance, the engineered cell (e.g., altered T cell, altered NK cell) would secrete one or more IL-12 variant polypeptide. The ability to secrete the one or more IL-12 variant peptide can be regulated in a contextual manner (e.g., turned on within the tumor microenvironment), for instance, by a synthetic NOTCH receptor.

In some embodiments, one or more nucleic acid encoding one or more IL-12 variant polypeptide is included within an oncolytic virus. In this instance, cells infected by the oncolytic virus would secrete one or more IL-12 variant polypeptide.

In some embodiments, one or more nucleic acid encoding one or more IL-12 variant polypeptide is administered systemically or locally (e.g., intratumorally) formulated as a lipid nanoparticle. In other embodiments one or more nucleic acids encoding one or more IL-12 variant polypeptide is electroporated intratumorally. In these scenarios, the IL-12 variant polypeptides are produced endogenously by the patients' own cells.

In some embodiments, the method of the present disclosure is useful for treating or preventing a tumor or cancer tumors that have lost surface expression of MHC class I; such as a tumor that has lost B2m, the MHC locus, or has mutations in other members of the antigen presentation and/or antigen loading complex, such as tapasin.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present disclosure and practice the claimed methods. The following working examples therefore are not to be construed as limiting in any way the remainder of the disclosure.

### Example 1: IL-12 Partial Agonists

Cytokine partial agonists are capable of selectively biasing the activation of cell populations and signaling pathways that possess differential activation thresholds. Partial agonists of human IL-12 were generated by mutating the cytokine to reduce affinity for its signaling receptors. These IL-12 variants create a range of sub-maximal signaling amplitudes relative to the wild-type molecule. While not being bound by scientific theory, it is believed that these partial agonists may have the effect of selectively activating desired cell populations that promote anti-tumor immunity while avoiding those the elicit toxicity.

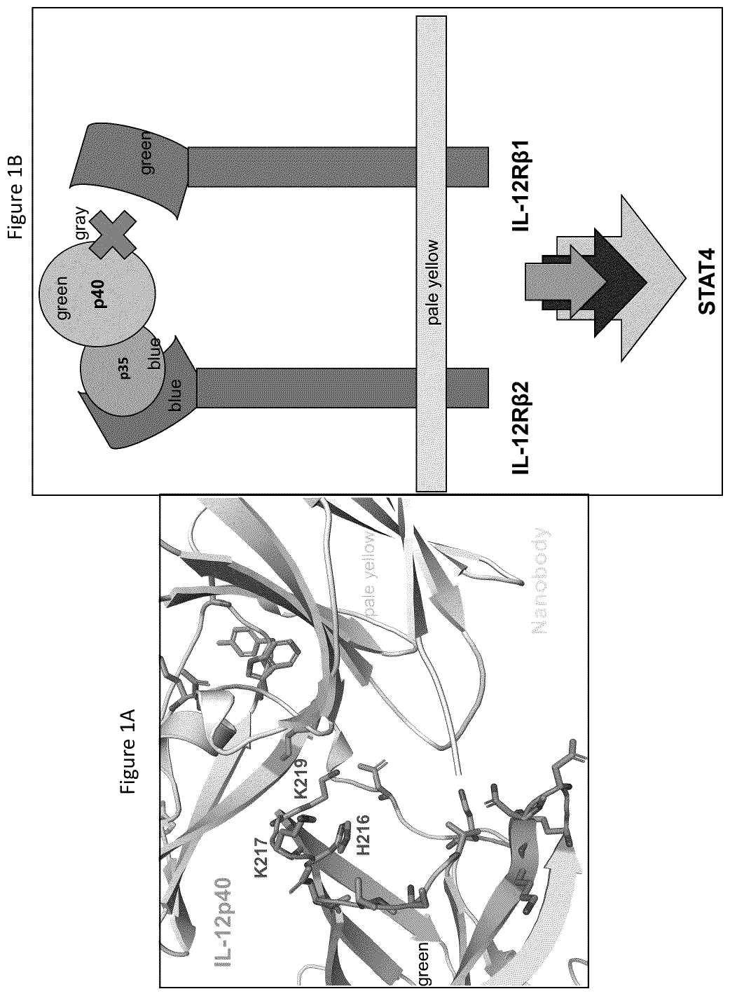

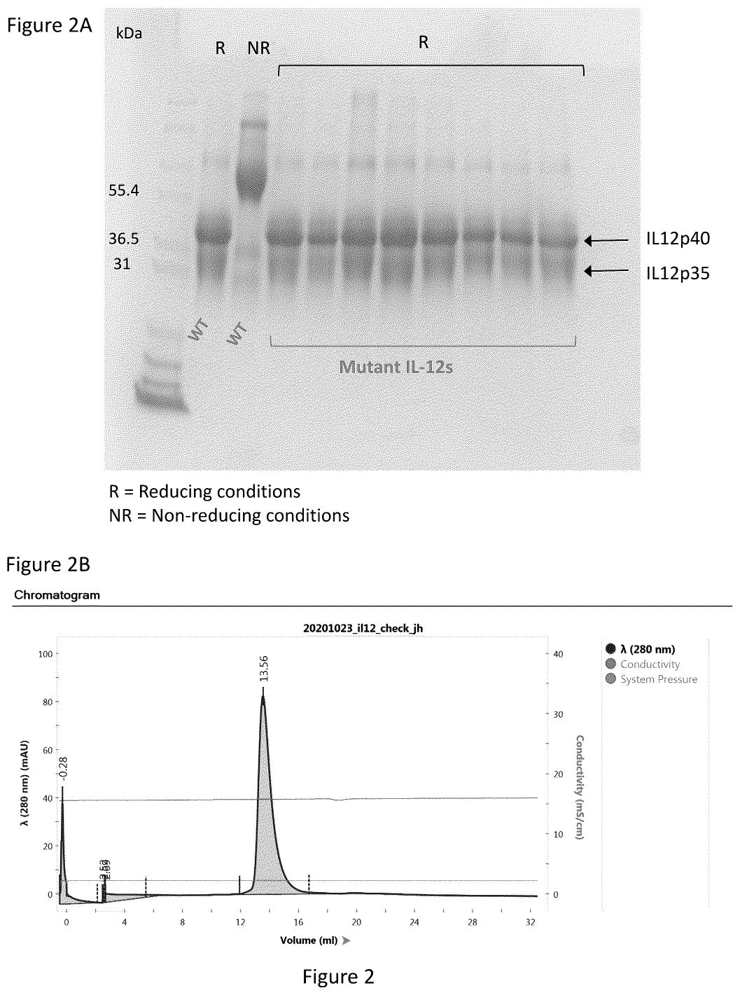

FIG. 1A and FIG. 1B depict models that predict residues at the IL-12Rβ1:IL-12p40 interface. FIG. 1A depicts a zoomed in model of IL-12p40 in complex with a neutralizing nanobody. Residues predicted to mediate interactions with the nanobody, and thus likely with IL-12Rβ1 as well, are depicted in blue (see written labels for color), IL-12p40 is depicted in green, and the nanobody is depicted in pale yellow. FIG. 1B depicts a schematic of the predicted interactions between IL-12, comprising IL-12p40 and IL-12p35 subunits, and IL-12Rβ2 and IL-12Rβ1. The gray “X” indicates the interface between IL-12Rβ1 and IL-12p40 that, if selectively disrupted, may reduce the recruitment of IL-12Rβ1 to the IL-12:IL-12Rβ2 complex and attenuate downstream STAT4 signaling. FIG. 2A and FIG. 2B provide results indicating protein expression and purification of wild-type (WT) IL-12 and mutant variants. FIG. 2A depicts exemplary results demonstrating comparable expression and mobility of WT and mutant IL-12 proteins. Proteins were separated on an SDS-PAGE gel and stained with Coomassie Brilliant Blue. FIG. 2B depicts exemplary results demonstrating purification of IL-12 and mutant variants. Proteins were isolated by nickel-NTA affinity chromatography and then further purified by size exclusion chromatography.

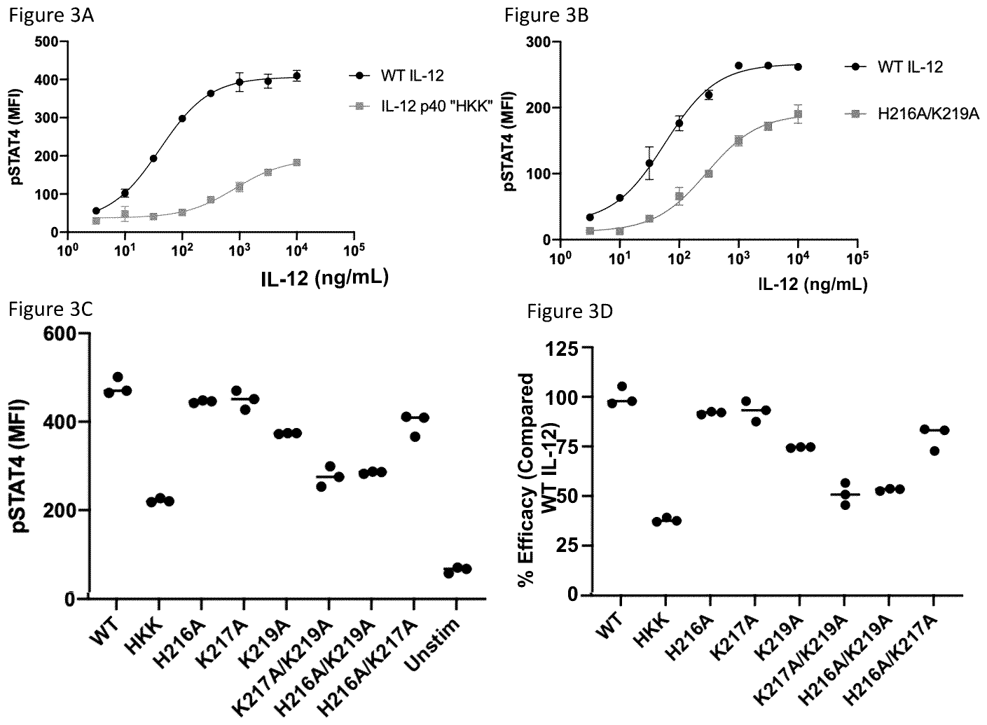

Based on the existing protein structure of IL-12p40 subunit bound to a neutralizing nanobody (PDB: 5MZV; FIG. 1A) it was examined whether surface exposed amino acids His216, Lys217, and Lys219 of IL-12p40 mediate interactions with IL-12Rβ1 (FIG. 1B). To test this hypothesis, each residue was mutated to an alanine in each possible unique combination (i.e. three single mutants, three double mutants, and one triple mutant), expressed in Expi293 cells (FIG. 2A) and purified using nickel-NTA affinity chromatography followed by size exclusion chromatography (FIG. 2B). Then, to test the agonism of IL-12Rβ1, phosphorylation of Signal transducer and activator of transcription 4 (STAT4) in human NK cells was measured by flow cytometry in the presence of increasing concentrations of WT IL-12 or the various mutants. FIG. 3A through FIG. 3D depict exemplary results demonstrating a graded response relative to WT depending upon the number and nature of the interface residues mutated to alanine residues. FIG. 3A provides exemplary results indicating reduced agonism of H216A/K217A/K219A triple mutant (HKK) as compared to WT IL-12. FIG. 3B provides exemplary results indicating agonism is reduced to a lesser extent in the H216A/K219A double mutant as compared to WT. FIG. 3C depicts exemplary results of single-point responses to all variants as compared to WT IL-12 and unstimulated cells. This suggests that a graded response that can be tailored to the particular level of agonism desired. FIG. 3D depicts the results of 3C as a percentage of agonism relative to WT. In all cases, human NK cells were stimulated with IL-12, a mutant variant, or left unstimulated, and phosphorylated STAT4 was measured by flow cytometry as a measure of IL-12 agonism.

As shown in FIG. 3A, mutation of all three residues (HKK) resulted in a dramatic increase in the effective concentration of IL-12 needed (EC₅₀=839 ng/mL) to reach 50% of maximal agonism (Eₘₐₓ≈192) as compared to wild-type (EC₅₀=40.8 ng/mL, Eₘₐₓ≈406), with a reduction in maximal agonism of ˜40-50%. Interestingly, the response could be graded depending upon the number and nature of mutations. As shown in FIG. 3B, H216A/K219A IL-12 had a reduction in maximal agonism of ˜70-75% (EC₅₀=290 ng/mL, Eₘₐₓ≈191) as compared to wild-type (EC₅₀=57.4 ng/mL, Eₘₐₓ≈267). Similarly, in single-point screen of all variants, the trend of a graded response held largely consistent, with the triple mutant having the most depressed response, double mutants having a slightly greater response, and single mutants having a response similar to wild-type (FIG. 3C-D). One exception appears to be H216A/K217A, which had a response similar to, if not greater than, K219A. Since K219A also had a lesser response as compared to H216A and K217A individually, this suggests that K219 may play a more critical role in mediating IL-12Rβ1:IL-12p40 interactions than H216 and K217.

Existing approaches to deliver IL-12, such as intratumoral injection, antibody fusions, and adoptive transfer of IL-12 expressing cells, use the wild-type protein and are therefore still likely to be toxic. The present approach is unique in that it utilizes an attenuated form of IL-12 that may be safer than wild-type when administered in tumor targeted or systemic delivery approach due to intrinsic differences in the cell types that can be activated. Further, to extend the half-life of these variants and improve efficacy, fusion to a range of stabilizing proteins could be employed, including heterodimeric Fc-fusion.

FIG. 4A through FIG. 4B depict exemplary results of IL-12 expressed as a bispecific heterodimeric Fc-fusion protein, a method of prolonging the in vivo half-life. FIG. 4A depicts a schematic of the bispecific heterodimeric Fc-fusion protein of IL-12 tested for IL-12 agonism. Interestingly, a bispecific heterodimeric Fc-fusion protein of IL-12 unexpectedly has attenuated agonism as compared to WT IL-12, but not as profoundly attenuated as the HKK triple mutant (FIGS. 4A and 4B). FIG. 4B depicts exemplary results demonstrating that the bispecific heterodimeric Fc-fusion protein of IL-12 unexpectedly has attenuated agonism as compared to WT IL-12, but not as profoundly attenuated as the HKK triple mutant as described in FIGS. 3A to 3D. Phosphorylated STAT4 was measured by flow cytometry as in FIGS. 3A to 3D.

FIG. 5A depicts a schematic of bivalent Fc fused to either p40 or p35 and co-expressed with the corresponding subunit to generate dimeric IL-12. FIG. 5B depicts a schematic of bivalent Fc fused to p35 which in turn is fused to p40 and is expressed as a single chain construct to form dimeric IL-12. FIG. 5C depicts a schematic of bispecific Fc “knob” fused to p35 which in turn is fused to p40, expressed as a single chain construct, and co-expressed with the corresponding bispecific Fc “hole” to form monomeric IL-12. FIG. 5D depicts a schematic of bispecific Fc “knob” fused to either p40 or p35 and co-expressed with the corresponding subunit and the corresponding bispecific Fc “hole” to form monomeric IL-12.

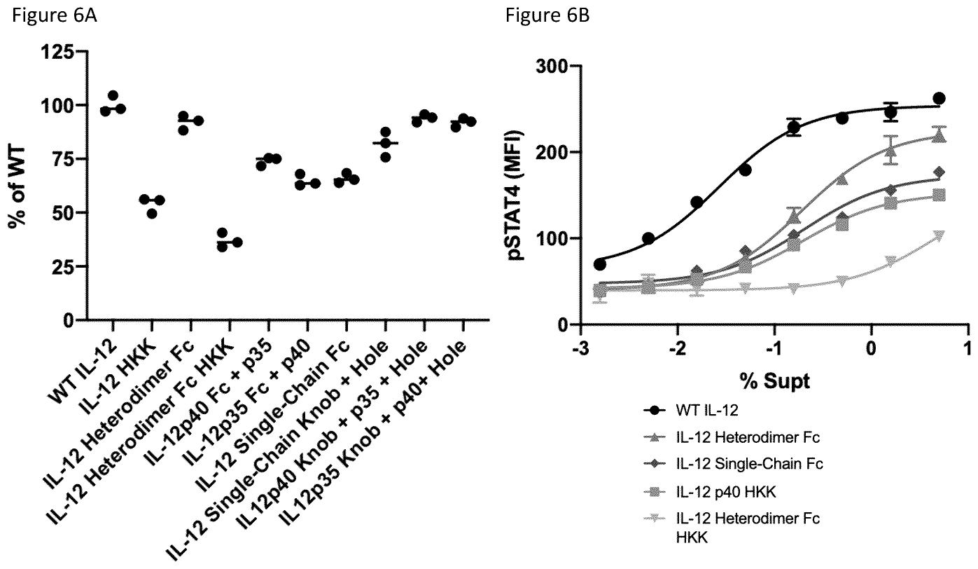

FIG. 6A depicts exemplary results of single-point responses to Fc-fusion variants as compared to WT IL-12 and IL-12 H216A/K217A/K219A triple mutant (HKK), demonstrating that the graded response as shown in FIGS. 3A to 3D can be further tuned via Fc-fusions. FIG. 6B depicts the effects of WT IL-12, IL-12 HKK and Fc-fusion variants on the phosphorylation of STAT4 in human NK cells in response to titrated amounts of supernatant. Proteins were expressed in Expi293 cells and cell culture supernatants containing the secreted proteins was used to stimulate NK cells. The x-axis depicts a titration of cell culture supernatants shown as a percentage of the total volume used to stimulate the NK cells.

As described above, a bispecific heterodimeric Fc-fusion protein of IL-12 unexpectedly has attenuated agonism as compared to WT IL-12, but not as profoundly attenuated as the HKK triple mutant (FIGS. 4A and 4B). Thus, it was believed that incorporating mutations at the IL-12Rβ1:IL-12p40 interface into the bispecific heterodimeric Fc-fusion protein of IL-12 (FIGS. 4A-4B) and/or any number of additional Fc-fusion IL-12 variants (FIGS. 5A-5D) would lead to further attenuation in agonism. Indeed, FIG. 6A and FIG. 6B demonstrate that HKK bispecific heterodimeric Fc-fusion IL-12 is further attenuated as compared to WT bispecific heterodimeric Fc-fusion IL-12 or IL-12 HKK.

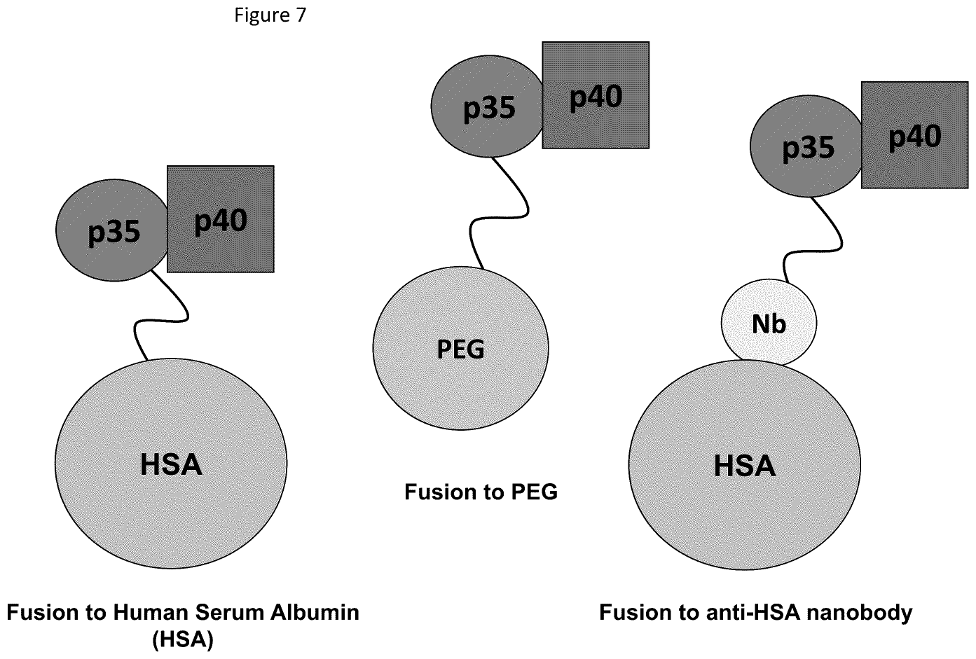

The additional Fc-variants, as diagrammed schematically in FIG. 5A through FIG. 5D, are also attenuated as compared to WT IL-12 and WT bispecific heterodimeric Fc-fusion IL-12. It is thus believed that incorporating mutations into these additional Fc-fusion variants will further attenuate agonism. Finally, it is believed that additional strategies may be employed to extend the in-vivo half-life of these variants and improve efficacy including, but not limited to, fusion to Human Serum Albumin (HSA), fusion to polyethylene glycol (PEG), or fusion to an anti-HSA nanobody (FIG. 7). For example, FIG. 7 provides a schematic providing additional exemplary methods of prolonging the in vivo half-life of a partial IL-12 agonist of the present disclosure. Thus, these partial agonists of IL-12 may represent a novel and safe approach to treat a range of cancer types, alone or in combination with other immunotherapies.

TABLE 1
IL-12 partial agonist amino acid sequences.

| SEQ | | |
|---|---|---|
| ID | Sequence | |
| NO: | Name | Amino Acid Sequence |
| 1 | WT IL- | MCHQQLVISWFSLVFLASPLVAIWELKKDVYVVELDWYPDAPGEMVVLT |
| | 12p40 | CDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQED |
| | | SACPAAEESLPIEVMVDAVHKLKYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 2 | WT IL- | MCPARSLLLVATLVLLDHLSLARNLPVATPDPGMFPCLHHSQNLLRAVS |
| | 12p35 | NMLQKARQTLEFYPCTSEEIDHEDITKDKTSTVEACLPLELTKNESCLN |
| | | SRETSFITNGSCLASRKTSFMMALCLSSIYEDLKMYQVEFKTMNAKLLM |
| | | DPKRQIFLDQNMLAVIDELMQALNFNSETVPQKSSLEEPDFYKTKIKLC |
| | | ILLHAFRIRAVTIDRVMSYLNAS |
| 3 | IL-12p40 | MCHQQLVISWFSLVFLASPLVAIWELKKDVYVVELDWYPDAPGEMVVLT |
| | H216A | CDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQED |
| | | SACPAAEESLPIEVMVDAVAKLKYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 4 | IL-12p40 | MCHQQLVISWFSLVFLASPLVAIWELKKDVYVVELDWYPDAPGEMVVLT |
| | K217A | CDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQED |
| | | SACPAAEESLPIEVMVDAVHALKYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 5 | IL-12p40 | MCHQQLVISWFSLVFLASPLVAIWELKKDVYVVELDWYPDAPGEMVVLT |
| | K219A | CDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQED |
| | | SACPAAEESLPIEVMVDAVHKLAYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 6 | IL-12p40 | MCHQQLVISWFSLVFLASPLVAIWELKKDVYVVELDWYPDAPGEMVVLT |
| | HK217 | CDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQED |
| | | SACPAAEESLPIEVMVDAVAALKYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 7 | IL-12p40 | MCHQQLVISWFSLVFLASPLVAIWELKKDVYVVELDWYPDAPGEMVVLT |
| | HK219 | CDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQED |
| | | SACPAAEESLPIEVMVDAVAKLAYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 8 | IL-12p40 | MCHQQLVISWFSLVFLASPLVAIWELKKDVYVVELDWYPDAPGEMVVLT |
| | KK | CDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQED |
| | | SACPAAEESLPIEVMVDAVHALAYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 9 | IL-12p40 | MCHQQLVISWFSLVFLASPLVAIWELKKDVYVVELDWYPDAPGEMVVLT |
| | HKK | CDTPEEDGITWTLDQSSEVLGSGKTLTIQVKEFGDAGQYTCHKGGEVLS |
| | | HSLLLLHKKEDGIWSTDILKDQKEPKNKTFLRCEAKNYSGRFTCWWLTT |
| | | ISTDLTFSVKSSRGSSDPQGVTCGAATLSAERVRGDNKEYEYSVECQED |
| | | SACPAAEESLPIEVMVDAVAALAYENYTSSFFIRDIIKPDPPKNLQLKP |
| | | LKNSRQVEVSWEYPDTWSTPHSYFSLTFCVQVQGKSKREKKDRVFTDKT |
| | | SATVICRKNASISVRAQDRYYSSSWSEWASVPCS |
| 10 | human | DKTHTCPPCPAPELLGGPSVFLFPPKPKDTLMISRTPEVTCVVVDVSHE |
| | IgG1 Fc | DPEVKFNWYVDGVEVHNAKTKPREEQYASTYRVVSVLTVLHQDWLNGKE |
| | | YKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSR |
| | | WQQGNVFSCSVMHEALHNHYTQKSLSLSPGK |
| 11 | Linker | GGGGGSGGGGSGGGGS |
| 12 | IL-12p40 | MEFGLSWVFLVALFRGVQSIWELKKDVYVVELDWYPDAPGEMVVLTCDT |
| | human | PEEDGITWTLDQSSEVLGSGKTLTIQVKEFGDAGQYTCHKGGEVLSHSL |
| | IgG1 Fc | LLLHKKEDGIWSTDILKDQKEPKNKTFLRCEAKNYSGRFTCWWLTTIST |
| | fusion | DLTFSVKSSRGSSDPQGVTCGAATLSAERVRGDNKEYEYSVECQEDSAC |
| | | PAAEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTDKTSAT |
| | | VICRKNASISVRAQDRYYSSSWSEWASVPCSGGGGSGGGGSGGGGSDKT |
| | | HTCPPCPAPELLGGPSVFLFPPKPKDTLMISRTPEVTCVVVDVSHEDPE |
| | | VKFNWYVDGVEVHNAKTKPREEQYASTYRVVSVLTVLHQDWLNGKEYKC |
| | | KVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWQQ |
| | | GNVFSCSVMHEALHNHYTQKSLSLSPGK |
| 13 | IL-12p35 | MEFGLSWVFLVALFRGVQSRNLPVATPDPGMFPCLHHSQNLLRAVSNML |
| | human | QKARQTLEFYPCTSEEIDHEDITKDKTSTVEACLPLELTKNESCLNSRE |
| | IgG1 Fc | TSFITNGSCLASRKTSFMMALCLSSIYEDLKMYQVEFKTMNAKLLMDPK |
| | fusion | RQIFLDQNMLAVIDELMQALNFNSETVPQKSSLEEPDFYKTKIKLCILL |
| | | HAFRIRAVTIDRVMSYLNASGGGGSGGGGSGGGGSDKTHTCPPCPAPEL |
| | | LGGPSVFLFPPKPKDTLMISRTPEVTCVVVDVSHEDPEVKFNWYVDGVE |
| | | VHNAKTKPREEQYASTYRVVSVLTVLHQDWLNGKEYKCKVSNKALPAPI |
| | | EKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHE |
| | | ALHNHYTQKSLSLSPGK |
| 14 | human | DKTHTCPPCPAPELLGGPSVFLFPPKPKDTLMISRTPEVTCVVVDVSHE |
| | IgG1 Fc | DPEVKFNWYVDGVEVHNAKTKPREEQYASTYRVVSVLTVLHQDWLNGKE |
| | “Knob” | YKCKVSNKALPAPIEKTISKAKGQPREPQVYTLPPSREEMTKNQVSLWC |
| | | LVKGFYPSDIAVEWESNGQPENNYKTTPPVLDSDGSFFLYSKLTVDKSR |
| | | WQQGNVFSCSVMHEALHNHYTQKSLSLSPGK |
| 15 | human | DKTHTCPPCPAPELLGGPSVFLFPPKPKDTLMISRTPEVTCVVVDVSHE |
| | IgG1 Fc | DPEVKFNWYVDGVEVHNAKTKPREEQYASTYRVVSVLTVLHQDWLNGKE |
| | “Hole” | YKCKVSNKALPAPIEKTISKAKGQPREPQVYTLPPSREEMTKNQVSLSC |
| | | AVKGFYPSDIAVEWESNGQPENNYKTTPPVLDSDGSFFLVSKLTVDKSR |
| | | WQQGNVFSCSVMHEALHNHYTQKSLSLSPGK |
| 16 | IL-12p40 | MEFGLSWVFLVALFRGVQSIWELKKDVYVVELDWYPDAPGEMVVLTCDT |
| | human | PEEDGITWTLDQSSEVLGSGKTLTIQVKEFGDAGQYTCHKGGEVLSHSL |
| | IgG1 Fc | LLLHKKEDGIWSTDILKDQKEPKNKTFLRCEAKNYSGRFTCWWLTTIST |
| | “Knob” | DLTFSVKSSRGSSDPQGVTCGAATLSAERVRGDNKEYEYSVECQEDSAC |
| | | PAAEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTDKTSAT |
| | | VICRKNASISVRAQDRYYSSSWSEWASVPCSGGGGGSGGGGSGGGGSDK |
| | | THTCPPCPAPELLGGPSVFLFPPKPKDTLMISRTPEVTCVVVDVSHEDP |
| | | EVKFNWYVDGVEVHNAKTKPREEQYASTYRVVSVLTVLHQDWLNGKEYK |
| | | CKVSNKALPAPIEKTISKAKGQPREPQVYTLPPSREEMTKNQVSLWCLV |
| | | KGFYPSDIAVEWESNGQPENNYKTTPPVLDSDGSFFLYSKLTVDKSRWQ |
| | | QGNVFSCSVMHEALHNHYTQKSLSLSPGK |
| 17 | IL-12p35 | MEFGLSWVFLVALFRGVQSRNLPVATPDPGMFPCLHHSQNLLRAVSNML |
| | human | QKARQTLEFYPCTSEEIDHEDITKDKTSTVEACLPLELTKNESCLNSRE |
| | IgG1 Fc | TSFITNGSCLASRKTSFMMALCLSSIYEDLKMYQVEFKTMNAKLLMDPK |
| | “Hole” | RQIFLDQNMLAVIDELMQALNFNSETVPQKSSLEEPDFYKTKIKLCILL |
| | | HAFRIRAVTIDRVMSYLNASGGGGGSGGGGSGGGGSDKTHTCPPCPAPE |
| | | LLGGPSVFLFPPKPKDTLMISRTPEVTCVVVDVSHEDPEVKFNWYVDGV |
| | | EVHNAKTKPREEQYASTYRVVSVLTVLHQDWLNGKEYKCKVSNKALPAP |
| | | IEKTISKAKGQPREPQVYTLPPSREEMTKNQVSLSCAVKGFYPSDIAVE |
| | | WESNGQPENNYKTTPPVLDSDGSFFLVSKLTVDKSRWQQGNVFSCSVMH |
| | | EALHNHYTQKSLSLSPGK |
| 18 | IL-12p40 | MEFGLSWVFLVALFRGVQSIWELKKDVYVVELDWYPDAPGEMVVLTCDT |
| | human | PEEDGITWTLDQSSEVLGSGKTLTIQVKEFGDAGQYTCHKGGEVLSHSL |
| | IgG1 Fc | LLLHKKEDGIWSTDILKDQKEPKNKTFLRCEAKNYSGRFTCWWLTTIST |
| | “Hole” | DLTFSVKSSRGSSDPQGVTCGAATLSAERVRGDNKEYEYSVECQEDSAC |
| | | PAAEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTDKTSAT |
| | | VICRKNASISVRAQDRYYSSSWSEWASVPCSGGGGGSGGGGSGGGGSDK |
| | | THTCPPCPAPELLGGPSVFLFPPKPKDTLMISRTPEVTCVVVDVSHEDP |
| | | EVKFNWYVDGVEVHNAKTKPREEQYASTYRVVSVLTVLHQDWLNGKEYK |
| | | CKVSNKALPAPIEKTISKAKGQPREPQVYTLPPSREEMTKNQVSLSCAV |
| | | KGFYPSDIAVEWESNGQPENNYKTTPPVLDSDGSFFLVSKLTVDKSRWQ |
| | | QGNVFSCSVMHEALHNHYTQKSLSLSPGK |
| 19 | IL-12p35 | MEFGLSWVFLVALFRGVQSRNLPVATPDPGMFPCLHHSQNLLRAVSNML |
| | human | QKARQTLEFYPCTSEEIDHEDITKDKTSTVEACLPLELTKNESCLNSRE |
| | IgG1 Fc | TSFITNGSCLASRKTSFMMALCLSSIYEDLKMYQVEFKTMNAKLLMDPK |
| | “Knob” | RQIFLDQNMLAVIDELMQALNFNSETVPQKSSLEEPDFYKTKIKLCILL |
| | | HAFRIRAVTIDRVMSYLNASGGGGGSGGGGSGGGGSDKTHTCPPCPAPE |
| | | LLGGPSVFLFPPKPKDTLMISRTPEVTCVVVDVSHEDPEVKFNWYVDGV |
| | | EVHNAKTKPREEQYASTYRVVSVLTVLHQDWLNGKEYKCKVSNKALPAP |
| | | IEKTISKAKGQPREPQVYTLPPSREEMTKNQVSLWCLVKGFYPSDIAVE |
| | | WESNGQPENNYKTTPPVLDSDGSFFLYSKLTVDKSRWQQGNVFSCSVMH |
| | | EALHNHYTQKSLSLSPGK |
| 20 | Linker 2 | GGGGSGGGGSGGGGSGGGGS |
| 21 | Linker 3 | GGGGSGGGGSGGGGS |
| 22 | Sc IL- | MEFGLSWVFLVALFRGVQSIWELKKDVYVVELDWYPDAPGEMVVLTCDT |
| | 12p40:IL- | PEEDGITWTLDQSSEVLGSGKTLTIQVKEFGDAGQYTCHKGGEVLSHSL |
| | 12p35:Fc | LLLHKKEDGIWSTDILKDQKEPKNKTFLRCEAKNYSGRFTCWWLTTIST |
| | | DLTFSVKSSRGSSDPQGVTCGAATLSVERVRGDNKEYEYSVECQEDSAC |
| | | PAAEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTDKTSAT |
| | | VICRKNASISVRAQDRYYSSSWSEWASVPCSGGGGSGGGGSGGGGSGGG |
| | | GSRNLPVATPDPGMFPCLHHSQNLLRAVSNMLQKARQTLEFYPCTSEEI |
| | | DHEDITKDKTSTVEACLPLELTKNESCLNSRETSFITNGSCLASRKTSF |
| | | MMALCLSSIYEDLKMYQVEFKTMNAKLLMDPKRQIFLDQNMLAVIDELM |
| | | QALNFNSETVPQKSSLEEPDFYKTKIKLCILLHAFRIRAVTIDRVMSYL |
| | | NASGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYASTY |
| | | RVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 23 | Sc IL- | MEFGLSWVFLVALFRGVQSRNLPVATPDPGMFPCLHHSQNLLRAVSNML |
| | 12p35:IL- | QKARQTLEFYPCTSEEIDHEDITKDKTSTVEACLPLELTKNESCLNSRE |
| | 12p40:Fc | TSFITNGSCLASRKTSFMMALCLSSIYEDLKMYQVEFKTMNAKLLMDPK |
| | | RQIFLDQNMLAVIDELMQALNFNSETVPQKSSLEEPDFYKTKIKLCILL |
| | | HAFRIRAVTIDRVMSYLNASGGGGSGGGGSGGGGSGGGGSIWELKKDVY |
| | | VVELDWYPDAPGEMVVLTCDTPEEDGITWTLDQSSEVLGSGKTLTIQVK |
| | | EFGDAGQYTCHKGGEVLSHSLLLLHKKEDGIWSTDILKDQKEPKNKTFL |
| | | RCEAKNYSGRFTCWWLTTISTDLTFSVKSSRGSSDPQGVTCGAATLSAE |
| | | RVRGDNKEYEYSVECQEDSACPAAEESLPIEVMVDAVHKLKYENYTSSF |
| | | FIRDIIKPDPPKNLQLKPLKNSRQVEVSWEYPDTWSTPHSYFSLTFCVQ |
| | | VQGKSKREKKDRVFTDKTSATVICRKNASISVRAQDRYYSSSWSEWASV |
| | | PCSGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYASTY |
| | | RVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 24 | human | MEFGLSWVFLVALFRGVQSRTGGGGGSGGGGSGGGGSDKTHTCPPCPAP |
| | IgG1 | ELLGGPSVFLFPPKPKDTLMISRTPEVTCVVVDVSHEDPEVKFNWYVDG |
| | “knob” | VEVHNAKTKPREEQYASTYRVVSVLTVLHQDWLNGKEYKCKVSNKALPA |
| | solo | PIEKTISKAKGQPREPQVYTLPPSREEMTKNQVSLWCLVKGFYPSDIAV |
| | | EWESNGQPENNYKTTPPVLDSDGSFFLYSKLTVDKSRWQQGNVFSCSVM |
| | | HEALHNHYTQKSLSLSPGK |
| 25 | human | MEFGLSWVFLVALFRGVQSRTGGGGGSGGGGSGGGGSDKTHTCPPCPAP |
| | IgG1 | ELLGGPSVFLFPPKPKDTLMISRTPEVTCVVVDVSHEDPEVKFNWYVDG |
| | “hole” | VEVHNAKTKPREEQYASTYRVVSVLTVLHQDWLNGKEYKCKVSNKALPA |
| | solo | PIEKTISKAKGQPREPQVYTLPPSREEMTKNQVSLSCAVKGFYPSDIAV |
| | | EWESNGQPENNYKTTPPVLDSDGSFFLVSKLTVDKSRWQQGNVFSCSVM |
| | | HEALHNHYTQKSLSLSPGK |
| 26 | Sc IL- | MEFGLSWVFLVALFRGVQSIWELKKDVYVVELDWYPDAPGEMVVLTCDT |
| | 12p40:IL- | PEEDGITWTLDQSSEVLGSGKTLTIQVKEFGDAGQYTCHKGGEVLSHSL |
| | 12p35:Fc | LLLHKKEDGIWSTDILKDQKEPKNKTFLRCEAKNYSGRFTCWWLTTIST |
| | “knob” | DLTFSVKSSRGSSDPQGVTCGAATLSVERVRGDNKEYEYSVECQEDSAC |
| | | PAAEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTDKTSAT |
| | | VICRKNASISVRAQDRYYSSSWSEWASVPCSGGGGSGGGGSGGGGSGGG |
| | | GSRNLPVATPDPGMFPCLHHSQNLLRAVSNMLQKARQTLEFYPCTSEEI |
| | | DHEDITKDKTSTVEACLPLELTKNESCLNSRETSFITNGSCLASRKTSF |
| | | MMALCLSSIYEDLKMYQVEFKTMNAKLLMDPKRQIFLDQNMLAVIDELM |
| | | QALNFNSETVPQKSSLEEPDFYKTKIKLCILLHAFRIRAVTIDRVMSYL |
| | | NASGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYASTY |
| | | RVVSVLTVLHQDWLNGKEYKCKVSNKALPAPIEKTISKAKGQPREPQVY |
| | | TLPPSREEMTKNQVSLWCLVKGFYPSDIAVEWESNGQPENNYKTTPPVL |
| | | DSDGSFFLYSKLTVDKSRWQQGNVFSCSVMHEALHNHYTQKSLSLSPGK |
| 27 | Sc IL- | MEFGLSWVFLVALFRGVQSRNLPVATPDPGMFPCLHHSQNLLRAVSNML |
| | 12p35:IL- | QKARQTLEFYPCTSEEIDHEDITKDKTSTVEACLPLELTKNESCLNSRE |
| | 12p40:Fc | TSFITNGSCLASRKTSFMMALCLSSIYEDLKMYQVEFKTMNAKLLMDPK |
| | “knob” | RQIFLDQNMLAVIDELMQALNFNSETVPQKSSLEEPDFYKTKIKLCILL |
| | | HAFRIRAVTIDRVMSYLNASGGGGSGGGGSGGGGSGGGGSIWELKKDVY |
| | | VVELDWYPDAPGEMVVLTCDTPEEDGITWTLDQSSEVLGSGKTLTIQVK |
| | | EFGDAGQYTCHKGGEVLSHSLLLLHKKEDGIWSTDILKDQKEPKNKTFL |
| | | RCEAKNYSGRFTCWWLTTISTDLTFSVKSSRGSSDPQGVTCGAATLSAE |
| | | RVRGDNKEYEYSVECQEDSACPAAEESLPIEVMVDAVHKLKYENYTSSF |
| | | FIRDIIKPDPPKNLQLKPLKNSRQVEVSWEYPDTWSTPHSYFSLTFCVQ |
| | | VQGKSKREKKDRVFTDKTSATVICRKNASISVRAQDRYYSSSWSEWASV |
| | | PCSGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYASTY |
| | | RVVSVLTVLHQDWLNGKEYKCKVSNKALPAPIEKTISKAKGQPREPQVY |
| | | TLPPSREEMTKNQVSLWCLVKGFYPSDIAVEWESNGQPENNYKTTPPVL |
| | | DSDGSFFLYSKLTVDKSRWQQGNVFSCSVMHEALHNHYTQKSLSLSPGK |
| 28 | Sc IL- | MEFGLSWVFLVALFRGVQSIWELKKDVYVVELDWYPDAPGEMVVLTCDT |
| | 12p40:IL- | PEEDGITWTLDQSSEVLGSGKTLTIQVKEFGDAGQYTCHKGGEVLSHSL |
| | 12p35:Fc | LLLHKKEDGIWSTDILKDQKEPKNKTFLRCEAKNYSGRFTCWWLTTIST |
| | “hole” | DLTFSVKSSRGSSDPQGVTCGAATLSVERVRGDNKEYEYSVECQEDSAC |
| | | PAAEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTDKTSAT |
| | | VICRKNASISVRAQDRYYSSSWSEWASVPCSGGGGSGGGGSGGGGSGGG |
| | | GSRNLPVATPDPGMFPCLHHSQNLLRAVSNMLQKARQTLEFYPCTSEEI |
| | | DHEDITKDKTSTVEACLPLELTKNESCLNSRETSFITNGSCLASRKTSF |
| | | MMALCLSSIYEDLKMYQVEFKTMNAKLLMDPKRQIFLDQNMLAVIDELM |
| | | QALNFNSETVPQKSSLEEPDFYKTKIKLCILLHAFRIRAVTIDRVMSYL |
| | | NASGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYASTY |
| | | RVVSVLTVLHQDWLNGKEYKCKVSNKALPAPIEKTISKAKGQPREPQVY |
| | | TLPPSREEMTKNQVSLSCAVKGFYPSDIAVEWESNGQPENNYKTTPPVL |
| | | DSDGSFFLVSKLTVDKSRWQQGNVFSCSVMHEALHNHYTQKSLSLSPGK |
| 29 | Sc IL- | MEFGLSWVFLVALFRGVQSRNLPVATPDPGMFPCLHHSQNLLRAVSNML |
| | 12p35:IL- | QKARQTLEFYPCTSEEIDHEDITKDKTSTVEACLPLELTKNESCLNSRE |
| | 12p40:Fc | TSFITNGSCLASRKTSFMMALCLSSIYEDLKMYQVEFKTMNAKLLMDPK |
| | “hole” | RQIFLDQNMLAVIDELMQALNFNSETVPQKSSLEEPDFYKTKIKLCILL |
| | | HAFRIRAVTIDRVMSYLNASGGGGSGGGGSGGGGSGGGGSIWELKKDVY |
| | | VVELDWYPDAPGEMVVLTCDTPEEDGITWTLDQSSEVLGSGKTLTIQVK |
| | | EFGDAGQYTCHKGGEVLSHSLLLLHKKEDGIWSTDILKDQKEPKNKTFL |
| | | RCEAKNYSGRFTCWWLTTISTDLTFSVKSSRGSSDPQGVTCGAATLSAE |
| | | RVRGDNKEYEYSVECQEDSACPAAEESLPIEVMVDAVHKLKYENYTSSF |
| | | FIRDIIKPDPPKNLQLKPLKNSRQVEVSWEYPDTWSTPHSYFSLTFCVQ |
| | | VQGKSKREKKDRVFTDKTSATVICRKNASISVRAQDRYYSSSWSEWASV |
| | | PCSGGGGSGGGGSGGGGSDKTHTCPPCPAPELLGGPSVFLFPPKPKDTL |
| | | MISRTPEVTCVVVDVSHEDPEVKFNWYVDGVEVHNAKTKPREEQYASTY |
| | | RVVSVLTVLHQDWLNGKEYKCKVSNKALPAPIEKTISKAKGQPREPQVY |
| | | TLPPSREEMTKNQVSLSCAVKGFYPSDIAVEWESNGQPENNYKTTPPVL |
| | | DSDGSFFLVSKLTVDKSRWQQGNVFSCSVMHEALHNHYTQKSLSLSPGK |
| 30 | IL-12p35 | MCPARSLLLVATLVLLDHLSLARNLPVATPDPGMFPCLHHSQNLLRAVS |
| | 8xHis | NMLQKARQTLEFYPCTSEEIDHEDITKDKTSTVEACLPLELTKNESCLN |
| | | SRETSFITNGSCLASRKTSFMMALCLSSIYEDLKMYQVEFKTMNAKLLM |
| | | DPKRQIFLDQNMLAVIDELMQALNFNSETVPQKSSLEEPDFYKTKIKLC |
| | | ILLHAFRIRAVTIDRVMSYLNASGGSHHHHHHHH |
| 31 | Signal | MCHQQLVISWFSLVFLASPLVA |
| | Peptide 1 | |
| 32 | Signal | MCPARSLLLVATLVLLDHLSLA |
| | Peptide 2 | |
| 33 | Signal | MEFGLSWVFLVALFRGVQS |
| | Peptide 3 | |
| 34 | WT IL- | IWELKKDVYVVELDWYPDAPGEMVVLTCDTPEEDGITWTLDQSSEVLGS |
| | 12p40 no | GKTLTIQVKEFGDAGQYTCHKGGEVLSHSLLLLHKKEDGIWSTDILKDQ |
| | signal | KEPKNKTFLRCEAKNYSGRFTCWWLTTISTDLTFSVKSSRGSSDPQGVT |
| | peptide | CGAATLSAERVRGDNKEYEYSVECQEDSACPAAEESLPIEVMVDAVHKL |
| | | KYENYTSSFFIRDIIKPDPPKNLQLKPLKNSRQVEVSWEYPDTWSTPHS |
| | | YFSLTFCVQVQGKSKREKKDRVFTDKTSATVICRKNASISVRAQDRYYS |
| | | SSWSEWASVPCS |
| 35 | WT IL- | RNLPVATPDPGMFPCLHHSQNLLRAVSNMLQKARQTLEFYPCTSEEIDH |
| | 12p35 no | EDITKDKTSTVEACLPLELTKNESCLNSRETSFITNGSCLASRKTSFMM |
| | signal | ALCLSSIYEDLKMYQVEFKTMNAKLLMDPKRQIFLDQNMLAVIDELMQA |
| | peptide | LNFNSETVPQKSSLEEPDFYKTKIKLCILLHAFRIRAVTIDRVMSYLNA |
| | | S |

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A composition comprising an IL-12 variant polypeptide, wherein said IL-12 variant polypeptide possesses sub-maximal signaling efficacy through its receptors relative to wild-type (WT) IL-12.

2. The composition of claim 1, wherein said IL-12 variant polypeptide comprises at least one mutation relative to WT IL-12.

3. The composition of claim 1, wherein said IL-12 variant polypeptide comprises a p35 subunit (IL-12p35) with or without a signal peptide and a p40 subunit (IL-12p40) with or without a signal peptide.

4. The composition of claim 2, wherein said IL-12p40 of said IL-12 variant polypeptide comprises at least one mutation selected from the group consisting of: H216X, K217X, and K219X, relative to SEQ ID NO: 1.

5. The composition of claim 4, wherein said IL-12p40 of said IL-12 variant polypeptide comprises at least one mutation selected from the group consisting of: H216A, K217A, and K219A, relative to SEQ ID NO: 1.

6. The composition of claim 3, wherein said IL-12p40 of said IL-12 variant polypeptide comprises an amino acid sequence selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 34.

7. The composition of claim 6, wherein said IL-12p35 of the IL-12 variant polypeptide comprises an amino acid sequence selected from the group consisting of SEQ ID NO: 2, SEQ ID NO: 30 and SEQ ID NO: 35.

8. The composition of claim 7, further wherein said IL-12p40, said IL-12p35, or a combination thereof is fused to at least one in vivo half-life extending fusion selected from a group consisting of: an IgG Fc domain, an IgG Fc variant domain, human serum albumin (HSA), polyethylene glycol (PEG), and an anti-HSA nanobody.

9. The composition of claim 8, wherein said IgG Fc domain comprises a human IgG1 domain comprising an amino acid sequence of SEQ ID NO: 10, and wherein said IgG Fc variant domain comprises at least one selected from a group consisting of: a human IgG1 Fc “knob” domain comprising an amino acid sequence of SEQ ID NO: 14; and a human IgG1 Fc “hole” domain comprising an amino acid sequence of SEQ ID NO: 14.

10. The composition of claim 9, wherein said IL-12 variant polypeptide comprises a bivalent homodimeric IgG Fc comprising at least two human IgG1 Fc domains.

11. The composition of claim 9, wherein said IL-12 variant polypeptide comprises a bispecific heterodimeric IgG Fc comprising at least one human IgG1 Fc “knob” and at least one IgG Fc “hole”.

12. The composition of claim 9, wherein said IL-12 variant polypeptide comprises a single chain bivalent homodimeric IgG Fc comprising said IL-12p40 fused to said IL-p35 via a linker and at least two human IgG1 Fc domains.

13. The composition of claim 9, wherein said IL-12 variant polypeptide comprises a single chain monomeric IL-12 comprising IL-12p40 fused to IL-p35 via a linker and a bispecific heterodimeric IgG Fc comprising at least one human IgG1 Fc “knob” and at least one IgG Fc “hole”.

14. The composition of claim 9, wherein said IL-12 variant polypeptide comprises dimeric IL-12 comprising said IL-12p40 and said IL-p35 and a bispecific heterodimeric IgG Fc comprising at least one human IgG1 Fc “knob” and at least one IgG Fc “hole.

15. A composition comprising one or more nucleic acid molecule encoding at least one IL-12p40 peptide selected from a group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, and SEQ ID NO: 9, and SEQ ID NO: 34.

16. The composition of claim 15, further comprising a nucleic acid molecule encoding IL-12p35 peptide comprising an amino acid sequence selected from a group consisting of SEQ ID NO: 2, SEQ ID NO: 30 and SEQ ID NO: 35.

17. The composition of claim 16, wherein said nucleic acid encoding said IL-12p40, said nucleic acid encoding said IL-12p35, or a combination thereof, further encodes a nucleic acid sequence encoding an in vivo half-life extending fusion selected from a group consisting of: an IgG Fc domain, an IgG Fc variant domain, human serum albumin (HSA), polyethylene glycol (PEG), and an anti-HSA nanobody.

18. A method of treating or preventing a disease or disorder in a subject in need thereof, comprising administering to said subject a composition comprising an IL-12 variant polypeptide, wherein said IL-12 variant polypeptide possesses sub-maximal signaling efficacy through its receptors relative to WT IL-12.

19. The method of claim 18, wherein said IL-12 variant polypeptide comprises a p35 subunit (IL-12p35) and a p40 subunit (IL-12p40).

20. The method of claim 19, wherein said IL-12p40 of said IL-12 variant polypeptide comprises an amino acid sequence selected from a group consisting of: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 34.

21. The method of claim 20, wherein said IL-12p35 of the IL-12 variant polypeptide comprises an amino acid sequence selected from a group consisting of SEQ ID NO: 2, SEQ ID NO: 30, and SEQ ID NO: 35.

22. The method of claim 21, further wherein said IL-12p40, said IL-12p35, or a combination thereof is fused to at least one in vivo half-life extending fusion selected from a group consisting of: an IgG Fc domain, an IgG Fc variant domain, human serum albumin (HSA), polyethylene glycol (PEG), and an anti-HSA nanobody.

23. The method of claim 18, wherein said disease or disorder is cancer.

24. The method of claim 23, further comprising administering to said subject at least one additional agent selected from a group consisting of: a chemical compound, a polypeptide, a peptide, a peptidomimetic, an antibody, a cytokine, a nucleic acid molecule (e.g., mRNA), a ribozyme, a small molecule chemical compound, and an antisense nucleic acid molecule.

25. The method of claim 24, wherein said at least one additional agent comprises one or more selected from a group consisting of: a cancer therapeutic agent or cancer immunotherapeutic agent.

26. The method of claim 18, comprising administering said IL-12 variant polypeptide via one or more mechanism selected from a group consisting of:
a) a lipid nanoparticle encapsulated mRNA molecule encoding said IL-12 variant polypeptide;
b) a viral vector expressing said IL-12 variant polypeptide; and
c) an engineered immune cell expressing said IL-12 variant polypeptide.

27. The method of claim 26, wherein said administration comprises one or more selected from a group consisting of:
a) Systemic administration; and
b) Local administration to at least one specific tissue.


SEQUENCE LISTING (35 sequences)

SEQ ID NO: 1 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 6 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 8 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 16 aa, synthetic construct
GGGGGSGGGGSGGGGS

SEQ ID NO: 12 - protein, 567 aa, synthetic construct
MEFGLSWVFLVALFRGVQSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 458 aa, synthetic construct
MEFGLSWVFLVALFRGVQSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 568 aa, synthetic construct
MEFGLSWVFLVALFRGVQSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 459 aa, synthetic construct
MEFGLSWVFLVALFRGVQSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 568 aa, synthetic construct
MEFGLSWVFLVALFRGVQSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 19 - protein, 459 aa, synthetic construct
MEFGLSWVFLVALFRGVQSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 20 aa, synthetic construct
GGGGSGGGGSGGGGSGGGGS

SEQ ID NO: 21 - protein, 15 aa, synthetic construct
GGGGSGGGGSGGGGS

SEQ ID NO: 22 - protein, 784 aa, synthetic construct
MEFGLSWVFLVALFRGVQSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSVERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 784 aa, synthetic construct
MEFGLSWVFLVALFRGVQSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 264 aa, synthetic construct
MEFGLSWVFLVALFRGVQSRTGGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 264 aa, synthetic construct
MEFGLSWVFLVALFRGVQSRTGGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 784 aa, synthetic construct
MEFGLSWVFLVALFRGVQSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSVERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 784 aa, synthetic construct
MEFGLSWVFLVALFRGVQSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 784 aa, synthetic construct
MEFGLSWVFLVALFRGVQSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSVERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 784 aa, synthetic construct
MEFGLSWVFLVALFRGVQSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 230 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGSHHHHHHHH

SEQ ID NO: 31 - protein, 22 aa, synthetic construct
MCHQQLVISWFSLVFLASPLVA

SEQ ID NO: 32 - protein, 22 aa, synthetic construct
MCPARSLLLVATLVLLDHLSLA

SEQ ID NO: 33 - protein, 19 aa, synthetic construct
MEFGLSWVFLVALFRGVQS

SEQ ID NO: 34 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 35 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS